SEQUENCE LISTING (9 sequences)

SEQ ID NO: 1 - protein, 5 aa, Pediococcus acidilactici
YGNGV

SEQ ID NO: 2 - protein, 34 aa, Lactococcus lactis
ITSISLCTPGCKTGALMGCNMKTATCNCSIHVSK

SEQ ID NO: 3 - DNA, 1469 bp, Lactococcus lactis
  source 1..1469: mol_type = other DNA
gacgaacgctggcggcgtgcctaatacatgcaagttgagcgatgaagattggtgcttgcaccaatttgaagagcagcgaacgggtgagtaacgcgtggggaatctgcctttgagcgggggacaacatttggaaacgaatgctaataccgcataacaactttaaacataagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacgggaggcagcagtagggaatcttcggcaatggacgaaagtctgaccgagcaacgccgcgtgggtgaagaaggttttcggatcgtaaaactctgttggtagagaagaacgttggtgagagtggaaagctcatcaagtgacggtaactacccagaaagggacggctaactacgtgccagcagccgcggtaatacgtaggtcccgagcgttgtccggatttattgggcgtaaagcgagcgcaggtggtttattaagtctggtgtaaaaggcagtggctcaaccattgtatgcattggaaactggtagacttgagtgcaggagaggagagtggaattccatgtgtagcggtgaaatgcgtagatatatggaggaacaccggtggcgaaagcggctctctggcctgtaactgacactgaggctcgaaagcgtggggagcaaacaggattagataccctggtagtccacgccgtaaacgatgagtgctagatgtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtg

SEQ ID NO: 4 - DNA, 1548 bp, Lactococcus lactis
  source 1..1548: mol_type = other DNA
tttatttgagagtttgatcctggctcaggacgaacgctggcggcgtgcctaatacatgcaagttgagcgatgaagattggtgcttgcaccaatttgaagagcagcgaacgggtgagtaacgcgtggggaatctgcctttgagcgggggacaacatttggaaacgaatgctaataccgcataacaactttaaacataagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttggtagagaagaacgttggtgagagtggaaagctcatcaagtgacggtaactacccagaaagggacggctaactacgtgccagcagccgcggtaatacgtaggtcccgagcgttgtccggatttattgggcgtaaagcgagcgcaggtggtttattaagtctggtgtaaaaggcagtggctcaaccattgtatgcattggaaactggtagacttgagtgcaggagaggagagtggaattccatgtgtagcggtgaaatgcgtagatatatggaggaacaccggtggcgaaagcggctctctggcctgtaactgacactgaggctcgaaagcgtggggagcaaacaggattagataccctggtagtccacgccgtaaacgatgagtgctagatgtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtgaagtcgtaacaaggtagccgtatcggaaggtgcggctggatcacctccttt

SEQ ID NO: 5 - DNA, 1409 bp, Bacillus subtilis
  source 1..1409: mol_type = other DNA
cgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacaccc

SEQ ID NO: 6 - protein, 541 aa, Lactococcus lactis
MNSHLNGVVNMKENWWQKTVVYQIYPRSFMDANGDGVGDLQGIISKLDYLEKLGIGAIWLSPVYQSPMDDNGYDISDYQAIADVFGTMSDMDELLLEAKKRNIQIVMDLVVNHTSDEHKWFVEARKSKDNAYRDYYIWADEPNALQSTFSGSAWEFDEESGQYFLHLFSKRQPDLNWENPQVHQEVYDMMNFWIDKGIGGFRMDVIDLIGKEIDQEITGNGPKLHEYLHEMNQATFGQKNLLTVGETWGATPEIAELYSDPKRQELSMVFQFEHITNAYLDEGEKWDKKEFSVSKLKEILAKWQALEKGWNSLFWNNHDLPRIVSNWGNDGKYRLKSAKAFAILLHLMKGTPYIYQGEELGMTNYPFESIEEVNDIESRNMFAERLAAGHSENEIMDSIRRVGRDNARTPMQWTAGENAGFTDGKPWLAVNPNHEEINADQAMSDPDSVFYTYQKLIELRKQHDWVIYGGFKLIDSEADVFAYLRTYKGKKYLVVANLSDEENQFKTGFVCRDLLIHNENFLPELSQIKLKAWEAFACEVE

SEQ ID NO: 7 - protein, 57 aa, Lactococcus lactis
MSTKDFNLDLVSVSKKDSGASPRITSISLCTPGCKTGALMGCNMKTATCHCSIHVSK

SEQ ID NO: 8 - protein, 566 aa, Bacillus subtilis
MRLTISRKFSLVFLTLILINLLVGGIGAFNMQHIIQKTDEINTKWIDGIKEITSINYLTEHLSSKEKDFLIFTDKSKMDTLDQEMNQILEDINQKLDSYEKTISNDKEQKLFEELQNEVNTYADIHAQIIESGRTNDMDKARGLLVQTEASFENMKKSVTQLVDFNKEGSNTAVKETKDVYHKGLIYTASLVAASIIISIFIWLYITRNIVKPIIRMKESANHIAEGDLSSDIEPLNSKDELGDLNEALQKMVGNLRDIVGYSKEISSRVLSSSQVLATATNETRSGSKHITETMNEMAEGSEQQAQDAVTIAESMNEFTESIDKAYNHGITISDTSQNVLELAVSGNENMDTSLQQMKTIHHIVQEAVHKVRSLEQHSQDINKLVQVINGIAEQTNLLSLNAAIEAARAGESGKGFAVVAEEVRKLADGVSDSVQDITRIVNGTQQEIYTVIEYLESSFTEVEKGTENLTDTGQAMQHIKQSVTHVADSIKEVTDGLKQLTNQSITINQSIENIASVSEESAAGIEETFSITEQSAHSMDQVLQNAEELEQLAKELNEKMNQFTI

SEQ ID NO: 9 - protein, 62 aa, Pediococcus acidilactici
MKKIEKLTEKEMANIIGGKYYGNGVTCGKHSCSVDWGKATTCIINNGAMAWATGGHQGNHKC

(19) United States
(12) Patent Application Publication
(10) Pub. No.: US 2026/0311575 A1
(43) Pub. Date: Oct. 8, 2026

(54) OPTIMIZED INDIVIDUALIZED PREBIOTIC COMPOSITIONS

(71) Applicant: ISOThrive LLC, Healdsburg, CA (US)

(72) Inventors: Lee Madsen, II, Manassas, VA (US); Jack Oswald, Healdsburg, CA (US); Sarah Stanley, Arlington, VA (US)

(21) Appl. No.: 19/013,907

(22) Filed: Jan. 8, 2025

Related U.S. Application Data
(63) Continuation of application No. 18/181,404, filed on Mar. 9, 2023.
Application No. 18/181,404 is a continuation of application No. 16/320,702, filed on Jul. 28, 2017, now abandoned.
(60) Provisional application No. 62/368,851, filed on Jul. 29, 2016.
(60) Provisional application No. 62/375,345, filed on Aug. 15, 2016.

(51) Int. Cl. G08B 7/06; G08B 5/38

(52) U.S. Cl. CPC G08B 7/066; G08B 5/38

(57) ABSTRACT

Methods are described herein for identifying and providing prebiotic compositions that are optimized for the health needs of individuals.

This application is a continuation of U.S. application Ser. No. 18/181,504, filed Mar. 9, 2023, which is a continuation of U.S. application Ser. No. 16/320,702, filed Jan. 25, 2019, which claims priority to U.S. national stage application filed under 35 U.S.C. § 371 from International Application Serial No. PCT/US2017/044387, filed on Jul. 28, 2017, and published as WO 2018/023003 on Feb. 1, 2018, which claims the benefit of priority to the filing dates of U.S. Provisional Application Ser. No. 62/368,851, filed on Jul. 29, 2016, and U.S. Provisional Application Ser. No. 62/375,345, filed on Aug. 15, 2016, the contents of each of which are specifically incorporated herein by reference in their entireties.

### INCORPORATION BY REFERENCE OF SEQUENCE LISTING

A Sequence Listing is provided herewith as an xml file, “2352290.xml” created on Jul. 19, 2023 and having a size of 14,496 bytes. The content of the xml file is incorporated by reference herein in its entirety.

### BACKGROUND

Intestinal microflora has a role in the health of human hosts. A growing body of evidence implicates digestive tract microbial dysbiosis in some chronic diseases and other health conditions (Hawrelak, J. A., & Myers, S. P. Altern Med Rev, 9(2): 180-197 (2004)). Intestinal (or gut) flora (microbiota) can include a set of microorganisms resident in the alimentary tube, and in an adult man, can include about 1014 bacteria with about 5000 to 10000 species of different bacteria.

Some researchers assert that probiotics can positively modulate the intestinal flora of mammalian hosts, for example, by restoring the balance of microorganisms in the gut. Such probiotics include non-pathogenic and non-toxic living organisms that can provide health benefits to the host. Examples of probiotic microorganisms that can be beneficial include Lactic Acid bacteria (LAB), that typically include lactobacilli (order Lactobacillus), and bifidobacteria (order Bifidobacteriales). It should be noted, however, that these indigenous probiotic organisms are not representative of the whole of the bacterial community residing in the digestive tract. For example, the greatest proportion in the typical western microbiome in the colon is constituted with the orders Bacteroidetes and Clostridiales. Also, the benefits of adding probiotic bacteria to the gut may not be realized unless the growth and metabolism of those bacteria are also fostered.

Prebiotics have recently been evaluated for beneficial effects on the health of mammalian hosts. A “prebiotic” includes one or more substances that, when ingested, is neither digested nor absorbed by the mammalian host, but is capable of stimulating the growth and/or the activity of bacteria of the intestinal flora, conferring benefits on health (Gibson G R, Roberfroid M B. Dietary modulation of the human colonic microbiota: introducing the concept of prebiotics. J Nutr. 1995 June; 125(6):1401-12. PMID). To date, attempts to positively impact the gut microbial community have mainly been based on trial and error. Product manufacturers and researchers have treated the gut microbiota community as a type of “black box,” where a prebiotic is administered that they hope will be beneficial, then the potential effects are (sometimes) checked.

### SUMMARY

The present invention represents the first systematic approach to identify the capabilities of an individual's gut microbial population, and then identify a specific mix of carbohydrates that will act as a prebiotic formulation that finely targets and manipulates the metabolism of one or more of the identified gut microorganisms to achieve specific health outcomes for the individual.

As illustrated herein, many types of microorganisms are naturally present in the intestines of humans, other mammals and birds, but some types of microorganisms are not necessarily present in the amounts, ratios, or activities that are optimal for the health of the mammalian or avian host, essentially because the prebiotic carbohydrate content of the host's diet is not adequate. The methods and compositions described here can adjust the growth (ratios or activities) of selected microorganisms by introducing prebiotic compositions that have been optimized for stimulation or inhibition of particular (selected) types of microorganisms. Screening methods are described for evaluating the intestinal microflora of human, mammalian, and/or avian subjects, so that optimal prebiotic compositions can be designed for the individual needs of particular subjects. The prebiotic compositions identified and produced as described herein are targeted to selected microorganisms, for example, so that the selected microorganisms can produce the types of nutrients and other beneficial agents that optimally benefit the human, mammalian, or avian subject. Instead of the “one size fits all” approach of currently available probiotic and prebiotic compositions, individualized prebiotic compositions are provided that can beneficially treat various diseases and conditions that an individual subject may suffer from.

Prebiotics are molecules, usually oligosaccharides or polysaccharides of plant origin, such as fructo-oligosaccharides (FOS) and inulin, capable of increasing the number and/or activity of lactic bacteria (lactobacilli) and/or of Bifidobacteria. Other prebiotic classes include galactooligosaccharides, xylooligosaccharides, and hemicellulosic fractions of various origin and glycosidic linkages types. The prebiotic class also includes oligo and polyglucans derivative of either cellulose or bacterial exopolysaccharide (EPS) and include but are not limited to oligodextrans with variable glycosidic linkage types and branching patterns.

### DESCRIPTION OF THE FIGURES

FIG. 1A-1B graphically illustrate the nisin content and carbohydrate profile of the culture medium after growth of L. lactis subsp. lactis NRRL B-1821 in media containing ISOThrive™ MIMO as a sole carbon source. FIG. 1A graphically illustrates the nisin content of the nisin standard (closed circles), a base broth (open squares), and the culture medium of Lactococcus lactis subsp. lactis NRRL-1821 that was antagonized with Wiessella viridescens NRRL B-1951. The nisin was detected by the tube-based bacteriocin assay described in Example 3. FIG. 1B graphically illustrates the carbohydrate profile (as detected by HPAEC-PAD showing the relative numbers of different chain lengths (nC) at different elution times) of L. lactis subsp. lactis NRRL B-1821 after growth in media containing ISOThrive™ MIMO as a sole carbon source. Trace 1: Pre-inoculum media. Trace 2: Media after 21 Hr fermentation. The detected peaks were: A, mannitol; B, L-arabinose (IS); C, glucose; D. unknown DP 2; E. leucrose; F. isomaltose; G, isomaltotriose; H, isomaltotetraose; I, maltose; J. panose (MIMO DP 3); and K-P, MIMO DP 4-9.

FIG. 2 graphically illustrates the metabolic profile (as detected by HPLC-RID) of L. lactis subsp. lactis NRRL B-1821 after growth in media with ISOThrive™ MIMO as a sole carbon source. Trace 1: Pre-inoculum media. Trace 2: media after 21 Hr fermentation. The peaks identified were: A, MIMO DP >3; B, panose; C, maltose; D, leucrose; E, unknown acid from media; F, glucose; G, mannitol; H, lactate; I, formate; J, acetate, and K, ethanol. nRIU refers to nano-refractive index units.

FIG. 3 illustrates the fermentative pathways for Lactococcus lactis [Oliveira et al. BMC Microbiology. 5:39 (2005)].

FIG. 4 illustrates overlaid HPAEC-PAD chromatograms of fermentation media containing ISOThrive™ MIMO with B. subtilis NRRL B-23049, at various time points of fermentation. Trace 1: pre-inoculum. Trace 2: media after 24 hr incubation. Trace 3 media after 44 hr fermentation. Trace 4: media after 72 hr fermentation. The components detected by HPAEC-PAD were: A, mannitol; B, unknown; C, L-arabinose (IS); D, glucose; E, isomaltotriose; F, isomaltotetraose; G, maltose, and H-M, PAN-type IMO (MIMO) DP 4-8.

FIG. 5 illustrates the metabolic profile (HPLC-RID) of B. subtilis NRRL B-23049 during fermentation in media containing ISOThrive™ MIMO as a sole carbon source. Trace 1: Pre-inoculation media. Trace 2: media after 24 Hr fermentation. Trace 3: media after 44 Hr fermentation. Trace 4: media after 72 Hr fermentation. The components detected in the media were: A, MIMO DP >3; B, panose; C, maltose; D, leucrose; E, glucose; F, mannitol; G, lactate; H, acetate, and I, unknown diol. nRIU refers to nano-refractive index units.

FIG. 6 graphically illustrates the rate of consumption by B. subtilis NRRL B-23049 of ISOThrive™ MIMOs with different degrees of polymerization (DP 3-7) at different time points in the fermentation. Top line: 0 hr fermentation. Second from the top line: 24 hr fermentation. Third line from the top: 44 hr fermentation. Bottom line: 72 hr fermentation. %/brix refers to grams of refractive dry solids (RDS) per 100 g of total material.

FIG. 7 illustrates overlaid HPAEC-PAD chromatograms of fermentation media containing ISOThrive™ MIMO with L. plantarum NRRL B-4496. Trace 1: pre-inoculation media. Trace 2: media after 34 hr fermentation. The components detected in the media were A, mannitol; B, L-arabinose (IS); C, unknown; D, glucose; E, leucrose; F, isomaltose; G, isomaltotriose; H. isomaltotetraose; I, maltose, and J-O, PAN-type IMO (MIMO) DP 3-8.

FIG. 8 illustrates the metabolic products (as detected by HPLC-RID) of L. plantarum NRRL B-4496 when ISOThrive™ MIMO is a sole carbon source. Trace 1: Pre-inoculation media. Trace 2: media after 34 Hr fermentation. The components detected were: A, MIMO DP >3; B, panose; C, maltose; D, leucrose; E, unknown acid from media; F, glucose; G, mannitol; H, lactate; I, formate; J, acetate, and K, ethanol. nRIU refers to nano-refractive index units.

### DETAILED DESCRIPTION

Optimized prebiotic compositions are described herein that can be designed for the individual needs of a mammalian or avian subject. Assay methods are also described herein for identifying individualized prebiotic compositions that can optimally balance the probiotic microorganism mixture within mammalian and/or avian subjects. In some cases, prebiotic compositions are provided for populations of subjects that all have similar health issues (e.g., vegetarians/vegans who may have vitamin B12 deficiencies, subjects with cancerous or precancerous conditions, subjects with gastrointestinal reflux, or subjects with irritable bowel syndrome).

The prebiotic compositions typically contain one or more types of carbohydrates. In some cases the carbohydrates in the prebiotic compositions are not significantly digested in the saliva, stomach or small intestine. In many cases a prebiotic is primarily intended to be digested or fermented in the large intestine, and should be resistant to digestion in the upper digestive tract. However, the upper gastrointestinal tract is also typically populated by bacteria and in some cases providing these organisms with appropriate prebiotic sustenance can also benefit the mammalian or avian subject. Hence, the prebiotic can be formulated for delivery to and/or metabolism within different parts of the gastrointestinal tract, including to the upper gastrointestinal tract, the lower gastrointestinal tract, or a combination thereof.

The carbohydrates can contain two or more sugar (monosaccharide) residues. For example, the carbohydrates can contain three or more sugar (monosaccharide) residues, or four or more sugar (monosaccharide) residues, or five or more sugar (monosaccharide) residues.

A variety of different sugar residues can be included in the carbohydrates. For example, the sugar residues can include any of the isomers of triose, tetrose, pentose, hexose, heptose, or octose monosaccharides, as wells as combinations thereof. In some cases, the sugar residues can include any of the α or β anomeric forms and/or any of the keto-forms, aldo-forms, furanose forms, pyranose forms, and/or linear forms of monosaccharides such as glucose, fructose, galactose, mannose, sorbose, psicose, fucose, allose, altrose, idose, gulose, talose, ribose, ribulose, xylose, xylulose, deoxyglucose, deoxyfructose, deoxygalactose, deoxymannose/rhamnose, deoxysorbose, deoxypsicose, deoxyallose, deoxyaltrose, deoxyidose, deoxygulose, deoxytalose, deoxyribose, deoxyribulose, deoxyxyulose, tagatose, hemicellulosic fractions, and combinations thereof.

The monosaccharides or sugars can be linked together by alpha or beta linkages. For example, the monosaccharides or sugars can be linked together by 1,2-linkages, 1,3-linkages, 1,4-linkages, 1,5-linkages, 1,6-linkages, 2,3-linkages, 2,4-linkages, 2,5-linkages, 2,6-linkages, or combinations thereof.

The composition can include one or more oligosaccharides such as fructo-oligosaccharides; beta-(2,6) oligofructan (levan); inulin; beta-(2,1) oligofructan; beta-1,2 oligosaccharides terminated with glucose; beta-(1,2)-galactooligosaccharides beta-(1,3)-galactooligosaccharides; beta-(1-4)-galactooligosaccharides; beta-(1,6) galactooligosaccharides; beta-(1,4) xylooligosaccharides; alpha-(1,2)-galactooligosaccharides; alpha-(1,3)-galactooligosaccharides; alpha-(1-4)-galactooligosaccharides; alpha-(1,6) galactooligosaccharides; beta-(1,4) xylooligosaccharides; xylooligoaccharides, hemicelluloses; arabinoxylan; galactomannan; guar gum; acacia gum; arabinogalactan, pectin, amylopectin, linkage permutations, or combinations thereof. Such oligosaccharides can be N- or O-substituted with fucose, sialic acid, sulfates, methyl groups, or amino groups. The oligosaccharides can have sugar residues that are missing one or more hydrogen atoms (e.g., dehydro and methylated sugars can be present in the oligosaccharides). Anhydro-end groups can also be present.

The prebiotic compositions described herein are designed to be metabolized by microorganisms that reside in specific parts of the gastrointestinal tract, including in the large intestine, or in the upper gastrointestinal tract.

Hence, in some cases the carbohydrates in the prebiotic compositions are digestible by microorganisms in the upper gastrointestinal tract.

In other cases, the carbohydrates in the prebiotic compositions are structurally designed to resist significant digestion by carbohydrate cleaving enzymes in the saliva, stomach, and small intestine of mammals and avians. For example, the percentage of linkages that can be cleaved by mammalian and avian digestive enzymes in the saliva, stomach and small intestine can be less than 40%, or less than 30%, or less than 20%, or less than 10%, of the total linkages between the monosaccharides of the carbohydrates.

Examples of enzymes that can be found in mammalian/avian saliva, mammalian/avian stomachs, and mammalian/avian small intestines include amylase, maltase, lactase, lysozyme, and sucrase-isomaltase. Amylase hydrolyzes alpha 1,4-linkages between starches and dextrins. Maltase (also called alpha-glucosidase, glucoinvertase, glucosidosucrase, maltase-glucoamylase, alpha-glucopyranosidase, glucosidoinvertase, alpha-D-glucosidase, alpha-glucoside hydrolase, alpha-1,4-glucosidase, glucoamylase, and alpha-D-glucoside glucohydrolase) converts maltose to glucose by cleaving the alpha-(1,4) linkages between the two glucose subunits. Lactase (also known as lactase-phlorizin hydrolase) cleaves the beta-(1,4) linkage of lactose to generate glucose and galactose. Lysozyme hydrolyzes 1,4-beta-linkages between N-acetylmuramic acid and N-acetyl-D-glucosamine residues in peptidoglycans. Sucrase-isomaltase is a digestive enzyme cohort present in the small intestine, in particular from the brush border of the small intestine. Disaccharidase, sucrase-isomaltase enzymes catalyze both the hydrolysis of the beta-(1,2) linkages of sucrose to yield fructose and glucose, and the hydrolysis of alpha-(1,6) linkages of oligosaccharides that are sufficiently small, in particular isomaltose, isomaltotriose, and isomaltotetraose to yield glucose. The ability of this enzyme to catalyze the hydrolysis of isomaltooligosaccharides decreases with the increase of substrate molecular weight.

Hence, while prebiotic carbohydrates designed to be metabolized in the large intestine may have a low number of alpha-1,4-linkages, most of the linkages in the prebiotic carbohydrate are not alpha-1,4-linkages. However, prebiotic compositions such as those designed to be metabolized in the stomach or small intestine may contain a significant proportion of alpha-1,4-linkages.

For example, the prebiotic carbohydrates that are selected for inclusion in prebiotic compositions by the methods described herein can have structures like those shown in Formula I.

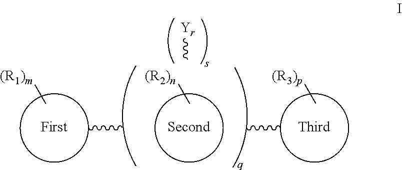

where:
   each First, Second, and Third ring is separately a three-atom, four-atom, five-atom, or six-atom heterocyclic ring with one or two oxygen, sulfur, or nitrogen heteroatoms;
   each Y is an optional monosaccharide or oligosaccharide with r monosaccharides, where each Y has a linkage ( [symbol] ) to a Second ring;
   each [symbol] is separately a linkage between First, Second, and Third ring subunits, as well as linkages between each Y monosaccharide or Y oligosaccharide and a Second ring;
   each m, n, and p is an integer separately selected from any of 2-5;
   q is an integer selected from any of 1-100;
   each r is an integer separately selected from 0-10;
   s is an integer selected from 0-20; and
   each R₁, R₂, and R₃ is separately selected from any of hydrogen, hydroxy, alkoxy, amino, carboxylate, aldehyde (CHO), phosphate or sulfate.

In some cases, one or more of the First, Second, or Third rings of Formula I is selected from a five-atom, or six-atom heterocyclic ring. In some cases, one or more of the First, Second, or Third rings of Formula I has an oxygen or nitrogen heteroatom. In some cases, one or more of the First, Second, or Third rings of Formula I has an oxygen heteroatom. In some cases, the Third ring can be a monosaccharide. For example, the Third ring can be a reducing monosaccharide. In some cases, the Third ring can be a glucose.

The linkages ( [symbol] ) between rings or monosaccharides of the carbohydrates of Formula I can be alpha or beta linkages. The linkages can be between different ring carbons of the such as 1,2-linkages, 1,3-linkages, 1,4-linkages, 1,5-linkages, 1,6-linkages, 2,1-linkages, 2,2-linkages, 2,3-linkages, 2,4-linkages, 2,5-linkages, 2,6-linkages, 3,1-linkages, 3-2, linkages, 3,3-linkages, or combinations thereof. However, the percentage of linkages in the carbohydrates of the prebiotic compositions that can be cleaved by mammalian and/or avian digestive enzymes in the saliva, stomach and small intestine is less than 20%, or less than 10%, of the total linkages between the First ring, Second rings, Third rings and Y groups. For example, the percentage of linkages in the carbohydrates of the prebiotic compositions that are alpha-(1,4) linkages can be less than 20%, or less than 10% of the total linkages between the First ring, Second rings, Third rings and Y groups.

In some cases, each m, n, or p can be an integer separately selected from any of 3-5. For example, for some carbohydrates of Formula 1 or II, each m, n, or p can be an integer separately selected from any of 4-5.

In some cases, the q variable is an integer is selected from any of 1-20. In some cases, the q variable is an integer is selected from any of 1-15, or 1-10.

In various cases, the value of q is typically larger than s. For example, in some cases the variable q can be an integer of from 2 to 15, or of from 2 to 10, or of from 2 to 7. However, in some cases s can be an integer of from 1 to 5, or of from 1 to 3, or of from 1 to 2.

The r variable defines the number of monosaccharides in the optional Y monosaccharide or oligosaccharide. For example, in some cases, the r variable can vary from about 0 to 10, or from about 0 to 7, or from about 0 to 5, or from about 0 to 3, or from about 0 to 1.

In some cases, the prebiotic compositions include maltosyl-isomaltooligosaccharides (MIMOs). “Maltosyl-isomaltooligosaccharides,” or MIMOs, or, by convention, “isomaltosyl-maltooligosaccharides” (IMOMs) refer to an oligosaccharide, an isomaltooligosaccharide glucan. In some cases, the MIMOs can have less than 40 degrees of polymerization, less than 30 degrees of polymerization, less than 20 degrees of polymerization, or less than 10 degrees of polymerization. MIMOs have a majority of α-(1->6) linkages but they can be terminated with an α-(1->4) linkage to the reducing-end (D-glucose). The α-(1->4) terminal group is comprised of maltose. Hence, a MIMO is called a maltosyl-isomaltooligosaccharide, or MIMO, or IMOM/IMOG, as per IUPAC convention. MIMOS can be produced by an acceptor reaction with either maltose or other isomaltooligosaccharide. An example of an MIMO with a single maltosyl linkage [—O-α-(1,4)-] linkage at the reducing end is maltosyl-isomaltotriose has the following chemical structure:

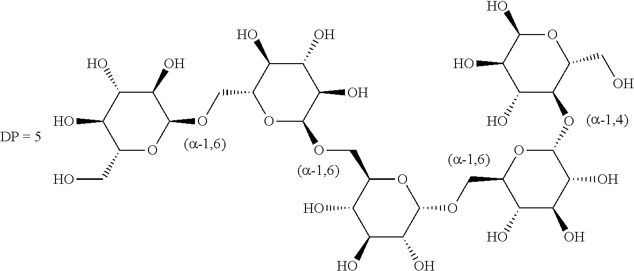 [SMILES: OC[C@H]1O[C@H](OC[C@H]2O[C@H](OC[C@H]3O[C@H](OC[C@H]4O[C@H](O[C@H]5[C@H](O)[C@@H](O)[C@@H](O)O[C@@H]5CO)[C@H](O)[C@@H](O)[C@@H]4O)[C@H](O)[C@@H](O)[C@@H]3O)[C@H](O)[C@@H](O)[C@@H]2O)[C@H](O)[C@@H](O)[C@@H]1O]

The prebiotic compositions can, for example, include maltosyl-isomaltooligosaccharides with a mass average molecular weight distribution of about 504 to 10,000 daltons, 640 to 10,000 daltons, 730 to 10,000 daltons, 504 to 7500 daltons, 504 to 5000 daltons, or 504 to 3000 daltons. In some cases, the mass average molecular weight distribution of the maltosyl-isomaltooligosaccharides can be about 730 to 900 daltons. The maltosyl-isomaltooligosaccharides in the compositions can in some cases contain more α-(1-6) glucosyl linkages than α-(1,2), α-(1,3), or α-(1,4) glucose linkages.

In some cases, the prebiotic compositions can have no detectable amounts of mannitol as detected by refractive HPAEC-PAD or HPLC-RID. In other cases, the prebiotic compositions can have some mannitol in them. For example, the compositions can have more than 3%/brix mannitol, or more than 4% %/brix mannitol, or more than 5% %/brix mannitol as detected by refractive HPAEC-PAD or HPLC-RID. For example, the amount of mannitol in the compositions can be less than 30%/brix mannitol, or less than 20%/brix mannitol, or less than 15%/brix mannitol or less than 12%/brix mannitol, or less than 10%/brix mannitol, or less than 9%/brix mannitol, or less than 8%/brix mannitol (e.g., 5-6%/brix) as detected by HPAEC-PAD or HPLC-RID.

When the compositions contain maltosyl-isomaltooligosaccharides there are generally no more than about 17 glucosyl units, or no more than about 16 glucosyl units, or no more than about 15 glucosyl units, or no more than about 14 glucosyl units, or no more than about 13 glucosyl units as detected by HPAEC-PAD or HPLC-RID.

In some cases, the compositions can have less than 2%/brix isomaltose, or less than 1%/brix isomaltose, or less than 0.5%/brix isomaltose, or less than 0.2%/brix isomaltose, or less than 0.1%/brix isomaltose as detected by HPAEC-PAD or HPLC-RID. In some cases, the compositions have no isomaltose, or levels below the detection limit (for example, as detected by HPAEC-PAD or HPLC-RID).

The compositions also can have less than 5%/brix glucose, or less than 4%/brix glucose, or less than 3%/brix glucose, or less than 2%/brix glucose, or less than 1%/brix glucose as detected by HPAEC-PAD or HPLC-RID.

The compositions also can have less than 5%/brix sucrose, or less than 4%/brix sucrose, or less than 3%/brix sucrose, or less than 2%/brix sucrose as detected by HPAEC-PAD or HPLC-RID.

The compositions also can have less than 4%/brix fructose, or less than 3%/brix fructose, or less than 2%/brix fructose, or less than 1%/brix fructose, or less than 0.5%/brix fructose, or less than 0.25%/brix fructose as detected by HPAEC-PAD or HPLC-RID.

The compositions can contain small or non-detectable quantities of organic acids such as lactic acid, acetic acid or formic acid. For example, the compositions can have less than 16%/brix lactic acid, acetic acid and formic acid; less than 3%/brix lactic acid, acetic acid and formic acid; less than 2%/brix lactic acid, acetic acid and formic acid; or less than 1%/brix lactic acid, acetic acid, and formic acid; or less than 0.5%/brix lactic acid, acetic acid, and formic acid; or less than 0.2%/brix lactic acid, acetic acid, and formic acid; or less than 0.1%/brix lactic acid, acetic acid, and formic acid as detected by HPAEC-PAD or HPLC-RID. In some cases, the compositions can have no organic acids such as lactic acid, acetic acid or formic acid, as measured by HPAEC-PAD or HPLC-RID.

In some cases, the prebiotic compositions can be any of those described in PCT application PCT/US2017/013957, filed Jan. 16, 2017 (claiming priority to U.S. Ser. No. 62/280,026 filed Jan. 18, 2016), both of which are incorporated herein by reference in their entireties.

The prebiotic compositions can also include plant dietary polysaccharides, including soluble polysaccharides, such as soluble hemicelluloses and celluloses that some types of microorganisms can metabolize. The breakdown products of such plant polysaccharides often feed beneficial microorganisms. For example, B. thetaiotaomicron can process some plant polysaccharides to provide products that foster E. rectale synthesis of butyrate.

### Methods of Selecting Prebiotics

Methods of identifying optimal prebiotic compositions for the individual needs of a subject can involve one or more of the following steps.

One or more samples (e.g. fecal samples, stomach samples, or upper gastrointestinal samples) can be obtained from a mammalian or avian subject. The diversity of microorganisms in the sample(s) can be determined. For example, RNA or rDNA can be isolated from a sample and the ribosomal RNA or ribosomal DNA sequences (e.g. 16S or 23S rRNA sequences) can be determined to identify what types of microorganisms reside in the gut of the subject who provided the sample. The types of microorganisms in the samples can also be determined by available microbiological methods, from sequencing other types of RNA and/or via sequencing of selected genomic segments or genes. Whole genomic sequencing can also be employed, for example, using shotgun (de novo) methods. In some cases, the numbers or proportions of types/classes of microorganisms can be determined by quantifying the classes of glycolytic enzymes encoded by the population of microorganisms in a sample. The types and proportions of the carbohydrate metabolizing enzymes in the population of sample microorganisms not only facilitates design of the prebiotic composition, but also can help with definitive identification of the species and strains of microorganisms in the sample. If further information is desired on the types of microorganisms in a sample, sample can be diluted, the microorganisms can be separated and then subcultured to evaluate what agents (e.g., bacteriocins, short chain fatty acids, vitamins, anti-cancer agents, antibiotics, neuromodulators, co-factors, toxins, or combinations thereof) the microorganisms can produce.

In some cases, the method can involve: (1) acquiring a fecal sample, stomach content sample, or an upper gastrointestinal sample, (2) sequencing to identify what types of microorganisms are in the sample (e.g., using one or more of the methods noted above), (3) identifying what enzymes break down carbohydrates for one or more (sometimes most) of the microorganisms in the sample, optionally identifying which carbohydrates are preferred by which microorganisms; (4) optionally identifying transport mechanisms to determine which carbohydrate break down residues may be used for metabolism, (5) identify what agents each organism is capable of producing, and combinations of (1) to (5).

One or more microorganisms is selected for growth or for inhibition of growth based upon one or more of its properties. One factor that is considered is an ability to synthesize helpful or unhelpful agents. Another factor is the physiological state of the subject. For example, the selection of a naturally present microorganism to increase or decrease the growth thereof can relate to analysis of whether the subject has one or more diseases or conditions, and a determination of which functions, properties, or agents a microorganism detected in the sample may provide to the subject.

For example, subjects may have one or more diseases or conditions such as cancer, pre-cancerous condition(s) or cancerous propensities, diabetes (e.g., type 2 diabetes), autoimmune disease(s), vitamin deficiencies, mood disorder(s), degraded mucosal lining(s), ulcerative colitis, digestive irregularities (e.g., Irritable Bowel Syndrome, acid reflux, constipation, or a combination thereof). For example, intestinal wall inflammation is a common health problem that is often related to microbial irritation of the intestinal wall, or even microbial digestion of intestinal wall components. If gut microorganisms do not have preferred substrates for growth they can digest secondary or less preferred substrates. See, e.g., Desai et al. Cell 167(5): 1339-53 (2016). For example, gut microorganisms can digest mucins that line the intestinal wall when preferred carbohydrate substrates are not available. Such digestion of mucins can weaken the intestinal wall to bacterial contact, and also lead to exposure of intestinal wall proteins and bacterial proteins to the immune system, which can initiate rounds of immune reactions and inflammation. Such problems can be reduced or obviated by ingestion of an appropriate prebiotic composition that contains one or more of the carbohydrates that are preferred by each intestinal microorganism.

The properties of the various microorganisms in the sample can be determined to identify which microbial functions/metabolites can be upregulated or downregulated to optimally serve the subject from which the sample was obtained. The properties of the various microorganisms in the samples are identified, for example, by reference to the teachings herein or other information. For example, the properties of the different microorganisms can include an ability to synthesize bacteriocins, short chain fatty acids (SCFAs), vitamins, anti-cancer agents, antibiotics, neuromodulators, co-factors, or combinations thereof. These properties can also be determined by reference to the genomic sequences of the microorganisms.

Carbohydrate preferences of one or more selected microorganisms (that has or have been detected in the sample) are identified. This can be done by identifying specific types of enzymes that are encoded by the genome(s) of the one or more microorganism(s). Because the identities of microorganisms in fecal or other samples can be identified with certainty, and the genomic sequences of such microorganisms are available, the identities of enzymes that digest one or more carbohydrates can be identified with certainty.

Carbohydrate preferences can be dictated by the ability of one or more microorganisms to synthesize enzymes that degrade those carbohydrates, and also by the enzymes or proteins that can transport certain carbohydrates into the microbial cell. An enzyme can be synthesized in response to the presence of a given substrate or in the absence of a preferred substrate. Glucose and glucose-containing oligosaccharides are often a preferred substrate for many microbial metabolic enzymes.

Carbohydrate preferences of a community of microorganisms can be predicted by identification of intracellular, extracellular, or periplasmic space enzymes that digest complex carbohydrate prebiotics to generate simpler saccharides that are readily used by various members of the microbial community. For example, carbohydrates can be processed in the periplasmic space of gram negative bacteria by a variety of enzymes that can signal carbohydrate preferences. A potential for such periplasmic space processing can be identified by interrogating whether carbohydrate transporters are encoded by the genome of the subject organism(s).

Examples of enzymes that can signal carbohydrate preferences include glycoside hydrolases (GH), glycosyl transferases, polysaccharide lyases (PL), carbohydrate esterases, dehydrogenases, carbohydrate transporters, and combinations thereof. The website at www.cazy.org lists types of carbohydrate active enzymes as well as providing information about the enzymes, such as which species has such enzymes, structural information about the enzymes, enzyme activities, substrate preferences, co-factor requirements. For example, the carbohydrate preferences of one or more selected microorganisms can be identified by determining whether one or more of the following types of enzymes are encoded by the genome(s) of the selected microorganism(s): alpha-glucosidases, beta-glucosidases, fructosidases, galactosidases, sucrases, sucrose-isomaltases, invertases, glucuronidases, glucose oxidases, maltases, amylases, isoamylases, beta-phosphoglucomutases, dextranases, pullulanases, mutanases, sialidases, glucosaminase, galactosaminases, xylanases, cellulases, and others. The carbohydrate preferences of one or more selected microorganisms can also be identified by determining whether carbohydrate transport proteins such as phosphoenolpyruvate: carbohydrate phosphotransferase system (PTS) are encoded by the genome(s) of the selected microorganism(s).

Methods for identifying the carbohydrate preferences of one or more selected microorganisms can include identification of particular enzyme sequences encoded within the genomes of those selected microorganisms by use of various databases of sequence information. Examples of databases with useful information include the databases and tools available from the National Center for Biotechnology Information (see website at www.ncbi.nlm.nih.gov/), the databases and tools available from UniProt (see website at www.uniprot.org/), the databases and tools available from ExPASY (see website at web.expasy.org), the databases and tools available from Swiss-Prot (see website at web.expasy.org/docs/swiss-prot), the databases and tools available from bacteria.ensembl.org, the databases and tools available from green genes (see website at greengenes.lbl.gov), the databases and tools available from the Microbial Genome Database (see website at mbgd.genome.ad.jp), or a combination thereof.

Some microorganisms can metabolize more than one type of carbohydrate, but there is typically a distinct hierarchy of carbohydrate preferences, where in the presence of two or more carbohydrates, the organism will shift to consuming the most preferred carbohydrate. By way of example, as a survival mechanism, a variety of bacteria have evolved to consume part of the gut mucosal lining when their preferred energy sources (specific types of carbohydrates, frequently containing mostly glucose) are not available. But when presented with a preferred type of carbohydrate, the bacteria shift to metabolizing the preferred carbohydrate source and away from the mucosal lining. One example is Bacteroides thetaiotaomicron, which is commonly found in the human colon and which can degrade many different complex carbohydrates (glycans). Although Bacteroides thetaiotaomicron can and will digest mucins that are found in the human intestine, it will also repress expression of genes involved in degrading lower priority carbohydrates (glycans) when higher priority (preferred) types of carbohydrates (glycans) are available. See, Rogers et al., Molec Microbiol 88(5): 876-90 (2013). Bacteroides thetaiotaomicron preferentially express the enzymes that degrade amylopectin and pectin galactin, but amylopectin is its more preferred carbohydrate source. Amylopectin is one of the two components of starch. Pectin is present in many types of fruits, for example, apples, plums, and citrus fruits are a good source of pectin. Purified amylopectin (e.g., from corn) can be obtained commercially from Sigma-Aldrich.

In addition, some microorganisms encode enzymes that degrade certain carbohydrates yet lack the intracellular transport mechanisms to utilize the breakdown products as an energy source. Such enzymes can be extracellular enzymes that are secreted into the surrounding environment. The products of such enzymatic action can feed other gut bacteria. In effect, the microorganisms that encode such enzymes are factories that behave as ‘chefs’ or ‘prep cooks’ for other bacteria that can make use of these breakdown products. For example, prebiotic compositions can be designed for these microorganism ‘prep cooks,’ which can then breakdown carbohydrates in the prebiotic compositions so the breakdown products can feed other gut microorganisms. Hence, if one type of gut microorganism produces useful products for a host subject, but if that microorganism does not encode the types of carbohydrate-metabolizing enzymes that can readily digest the carbohydrates in a prebiotic composition, the prebiotic composition can be designed to provide carbohydrates to a ‘prep cook’ microorganism that supplies the enzymes to breakdown the carbohydrates and produce products that feed, and thus increase the population and/or metabolic activity of other selected microorganisms.

With consideration for the hierarchy of carbohydrate preferences in the subpopulations of microorganisms selected for modulation, a specific carbohydrate profile is identified for a prebiotic composition that when delivered to the gut will increase the population, activity, and/or robustness of the targeted organism(s), or alternatively, will reduce the carbohydrate availability to organisms populations we wish to reduce (and/or in some cases, accomplish both at the same time to achieve a desired outcome).

Certain organisms are carbohydrate ‘finicky,’ and others are more carbohydrate ‘omnivorous.’ A carbohydrate preference hierarchy can be determined so that a preferred formulation of prebiotic carbohydrates can be manufactured that targets one or more gut microorganisms with specificity. The carbohydrate ‘finicky’ microorganisms can be provided with the types of carbohydrates that foster their metabolism and growth, while other carbohydrates in the formulation are available for the carbohydrate ‘omnivorous’ microorganisms. If inhibition of one or more microorganism is desired, the types of carbohydrates that would normally be metabolized by those microorganisms can be reduced or eliminated from the formulation. Hence, the growth or activities of different organisms can be manipulated by providing optimized, individualized prebiotic compositions to a subject.

The prebiotic carbohydrate compositions can be formulated to target specific health issues of individuals. For example, vegetarians often need vitamin B12 supplements due to the lack of meat consumption in their diet. The needs of such vegetarians can be served by ingestion of a prebiotic carbohydrate formulation that can upregulate the activity or growth of microorganisms in the gut with the capability of producing vitamin B12. For a specific individual host subject, identifying the organisms present in the gut that are capable of producing vitamin B12, as well as the carbohydrate preference hierarchy of those organisms and any energy transporters that may facilitate growth and metabolism by those organisms, determines the blend of prebiotic carbohydrates that would not only optimally foster microorganisms for vitamin B12 production, but would also balance the population of microorganisms in the gut.

For example, there may also be a need to increase butyrate production to inhibit or reduce “leaky gut” syndrome and the inflammatory conditions that are often associated with intestinal and other disease states. The prebiotic formulation can also provide carbohydrates to specific types of microorganisms that are capable of producing both vitamin B12 as well as small chain fatty acids (SCFAs) and/or butyrate. Hence, the methods and formulation described herein can be tailored to the needs of each unique individual.

In another example, vitamin B12 deficiencies may be combated in populations of vegetarians/vegans by analysis of collected bacterial populations from a wide swath of vegetarians/vegans so that the types of bacteria found in these vegetarians/vegans are determined and a blend of carbohydrates is made that would target all of the potential vitamin B12 producing bacteria that are likely to exist in any given host. This approach is less individualized yet can still be effective at achieving an amelioration of a targeted condition.

The probiotic formulations can be adjusted from time to time to meet newly developing health issues. For example, in the event of a diagnosis of colon cancer, the prebiotic compositions can be formulated to foster the growth and/or metabolic activity of specific microorganisms that can produce bacteriocin(s) and other useful factors. Such bacteriocins and other factors can treat or inhibit the growth and metastasis of specific cancer(s). Once the cancer had been eradicated, the portion of the prebiotic composition that was intended to produce higher than normal levels of bacteriocins, could be reduced to provide a lower maintenance level of cancer preventative protection.

As illustrated in the Examples provided herein, Lactococcus lactis is present at low levels and is stable in the baseline average population of microbiota in the human gut. The population of Lactococcus lactis in a human test group (N=13) increased by a factor of ten within 6-12 weeks of a trial where the human subjects consumed a specific type of prebiotic—maltosyl-isomaltooligosaccharides (MIMOs). The enhanced growth of this organism was predicted to occur by virtue of its genealogical encoding for the expression of DexA and DexB encoding for oligo-1,6-glucosidase and 1,6-glucosidase. The expression of oligo-1,6-glucosidase and 1,6-glucosidase was confirmed via in-vitro fermentation by Lactococcus lactis subsp. lactis NRRL B-1821 using MIMO (ISOThrive™) as a sole carbon source. Analysis described herein shows that Lactococcus lactis has a weighted prebiotic index (PrbI) for MIMO of at least 60, or at least 83. The utility of such a prebiotic index was demonstrated because MIMO did in fact stimulate the growth of Lactococcus lactis in the gut by ten-fold. Broth from fermentation of Lactococcus lactis subsp. lactis NRRL B-1821 contains beneficial bacteriocins, as illustrated by antimicrobial activity assays, and as measured via bio-assays including disk and tube-based methods which are described herein (see, e.g., Example 3).

The Examples (e.g., Example 8) provided herein also show that administration of a prebiotic can reduce or eliminate gastrointestinal reflux in a subject. Gastroesophageal reflux disease (GERD) is a common digestive disorder with symptoms including heartburn and dysphagia, progressing to more severe complications in some patients. GERD can resolve following dietary or lifestyle changes, but can also be treated pharmacologically. To date, ingested prebiotic soluble fiber has not been reported to improve symptoms. On the contrary, some types of soluble fiber, such as fructooligosaccharide (FOS), have been reported to worsen symptoms.

It has become increasingly apparent that bacteria can play a role in the pathogenesis of GERD (Yang et al. 2012). Throughout the gastrointestinal tract, a mucosal barrier defends the local epithelium against local ambient aggressive factors. The characteristics of this barrier in the distal third of the esophagus, as well as in other areas of the gastrointestinal tract, are influenced by the local microbiome (Harris et al. 2015). More than 100 commensal species of bacteria reside in the distal esophagus and recent discussions of the pathophysiology of GERD have addressed the importance of the microbiome intrinsic to the distal esophagus and LES (Yang et al. 2014).

The current proposed pathophysiology of GERD is as follows: Multiple factors, including dietary changes and antibiotic use, alter the local microbiome from a symbiotic relationship to a dysbiotic or potentially pathogenic one. This dysbiotic state has been grossly characterized as a change in the esophageal microbiome from predominantly Gram-positive organisms to a majority of Gram-negative species (Yang et al 2009). This transition alters the properties of the bacterial biofilm (Mathias and Corthesy 2011), including its permeability. This, in turn, can negatively affect the robustness of the mucosal barrier, exposing the esophageal epithelium to pathogenic bacteria, gastric refluxate, and swallowed bacterial toxins.

In addition, in the microbial dysbiotic state associated with GERD, the Gram-negative bacteria produce endotoxins such as lipopolysaccharides (LPS), which are presented on the outer bacterial membrane. These endotoxins upregulate expression of proinflammatory cytokines (Yang et al 2012) and trigger nitric oxide-induced relaxation of the LES (Fan et al. 2001), hindering its ability to close sufficiently to prevent gastric reflux. LPS can also delay gastric emptying (Calatayud et al 2002) and produce neuromuscular changes (Rieder et al. 2007) in the LES that decrease its resistance to gastric pressure (Fan et al 2001), both of which promote esophageal reflux.

As described herein, orally ingested isomaltooligosaccharides can selectively increase populations of certain Gram-positive organisms in the colon, including Bifidobacterium and Lactobacillus spp. (Yen et al 2011). Lactobacillus species found in the mouth (Badet and Thebaud 2008) and distal esophagus (Pei et al 2004) can metabolize soluble fiber, including maltosylisomaltooligosaccharide (MIMO). Accordingly, MIMO might increase the populations of specific Lactobacillus species in the upper GI tract and distal esophagus. It is conceivable that more abundant Gram-positive bacteria could produce elevated levels of bacteriocins toxic to fungi, Gram-negative bacteria such as Listeria spp., and other pathogenic organisms (Smaoui et al. 2010). For example, Lactobacillus salivarius, a common inhabitant of the human oral cavity, produces a bacteriocin that inhibits the growth of Campylobacter jejuni (Stern et al 2006), a Gram-negative S-shaped rod responsible for many cases of bacterial gastroenteritis worldwide. Consistent with a causative role of this genus in these disorders, Macfarlane et al. (2007) reported that Campylobacter species were detected in the majority of Barrett's esophagus (BE) patients but not in healthy controls. Furthermore, 16S ribosomal RNA gene sequencing and fluorescence microscopic examination of the BE patients revealed colonization of the esophageal mucosa by Campylobacter. Similarly, Blackett et al. (2013) reported an increased abundance of Campylobacter in patients with GERD and BE, with Campylobacter concisus predominating. Collectively, these observations imply that dietary supplementation with MIMO could help repopulate the LES microbiome with beneficial Gram-positive organisms and shift the microbial balance away from the pathogenic dysbiotic state associated with GERD.

These and other experimental results show that the methods described herein identify which organisms are natively present in the human gut, as well as which of these organisms can consume a given prebiotic (e.g., based on genes encoding the appropriate hydrolase enzymes). This application also demonstrates that the population of the bacteria natively present in the gut can be manipulated in a pre-determined way via introduction (administration) of an individualized prebiotic composition. The prebiotic composition can be selected to favor specific groups of bacteria that naturally produce particular types of beneficial agents (e.g., bacteriocins, vitamins, anti-cancer agents, antibiotics, short chain fatty acids (SCFAs), neuromodulators, co-factors, and combinations thereof). The population of the bacteria present in the gut can be monitored during the course of intervention so that the prebiotic composition can be tailored to balance the types of bacteria in the gut as desired. Similarly, the prebiotic composition may be a tailor-made cocktail of various prebiotic compositions that is specific for the microbiome of an individual person.

In some cases the methods for providing an optimized prebiotic composition to a subject can involve: identifying what health issues a subject may have (e.g. B12 deficiency, biopsied and thus identified colon cancer, Clostridium difficile overgrowth, etc.; sequence nucleic acids from the subject's sample; identify microbial species in the sample (and optionally identify the relative numbers or ratios of different types of microbial species in the sample); cross-reference identified species vs. genes that can produce the agent(s) that can ameliorate the subject's health issues; provide at least one prebiotic composition that can foster (growth and/or metabolism of) microbial species that can produce the agents that can ameliorate the subject's health issues.

The prebiotic composition can be administered to the subject. After ingestion of the prebiotic composition for at least a week, or at least two weeks, or at least three weeks or at least a month, or at least five weeks, or at least 6 weeks, or at least two months, another sample can be evaluated to identify what microbial species are present and in what amounts (ratios or activity). Any changes in the types, diversity, or ratios/activity of microbial species can be correlated with the health or clinical status of the subject.

The amounts or types of carbohydrates in the prebiotic compositions can also be varied to address any new health issues or to improve the composition formulation in any way.

In some cases the methods for selecting an optimized prebiotic carbohydrate composition can include a step-wise design of a prebiotic composition to include carbohydrate substrates that will first obviate common intestinal problems and then address the particular needs of a subject. For example, (1) a baseline formulation can be identified that includes substrates for organisms that can consume mucin or gut lining components, (2) then carbohydrate substrates are identified that address specific health concerns (e.g., by including substrates for gut microbes that can produce bacteriocins, vitamins, short chain fatty acids (SCFAs), anti-cancer agents, neuromodulators, co-factors (e.g., NAD, CAMP, etc.), and combinations thereof), (3) identify preferred carbohydrate substrates for each organism detected in a subject's fecal or other samples, and (4) evaluate a proposed prebiotic composition against the balance of the bacterial population in a subject's sample and adjust the types and amounts of carbohydrates in the composition to reduce and/or minimize carbohydrate competition for the organisms targeted for activity change. These steps are described in more detail below

### Identification of a Baseline Formulation

A baseline healthful mixture would include substrates in sufficient quantities to match no less than the second to least preferred substrate so that all organisms would refrain from mucin consumption and gut barrier degradation. This assumes, and is backed by DNA sequence analysis, that all commensal bacteria have gut mucin as their least preferred substrate.

### Health Changes by Activity Modification

After determining what health conditions can be adjusted by specific organism metabolites, the organisms that need modification of population size and/or metabolic activity can be chosen. The choice of prebiotic carbohydrate formulation to achieve a change in outcome is determined by (a) sorting all bacteria in order of the fewest encoded enzymes (i.e. “most carbohydrate finicky”) to the most encoded enzymes (i.e. “most carbohydrate omnivorous) and then by their preferred substrates from most to least preferred. Next, the subset of organisms that need activity adjustment are (a) sorted in order of the fewest encoded enzymes (i.e. “most carbohydrate finicky”) to the most encoded enzymes (i.e. “most carbohydrate omnivorous) and then by their preferred substrates from most to least preferred.

### Draft Carbohydrate Mix

When increasing activity is desired, an initial draft carbohydrate mix is determined and would include the most preferred substrate for each organism whenever possible. When doing so would create a conflict between organisms because of an overlap of preferred substrate, additional amounts of the common substrate would be included in the mix. Alternatively, in the event that one or more organisms are more “omnivorous” a substrate that is less than the most preferred can be substituted so that each organism is able to perform at a sufficient level of efficiency.

Prebiotic compositions can also be designed for microorganism (e.g. ‘prep cook’ microorganisms) that breakdown carbohydrates in the prebiotic compositions where the breakdown products can feed other gut microorganisms. Hence, if one type of gut microorganism produces useful products for a host subject, but that microorganism does not encode the types of carbohydrate-metabolizing enzymes that can readily digest the carbohydrates in a prebiotic composition, the prebiotic composition can be designed to provide carbohydrates to a ‘prep cook’ microorganism that supplies the enzymes to breakdown the carbohydrates to produce products that feed, and thus increase the population and/or metabolic activity of other selected microorganisms.

### Final Carbohydrate Mix

The draft mix is evaluated against the balance of the bacterial population and adjusted as needed so as to reduce and/or minimize carbohydrate competition for the organisms targeted for activity change. In general, for all organisms that are more omnivorous and whenever it is possible to include a more preferred substrate, these organisms will be “distracted” and not compete for the energy sources targeted for the organisms be manipulated.

The relative amounts of each substrate can be calculated by using (a) the starting relative populations and (b) rate of metabolic activity of each organism and these amounts can be modified to include projected increases/decreases in population of the organisms targeted to achieve a desired change in microbial gut activity. This process is illustrated in the charts shown below.

### Bacterium A Carbohydrate Consumption Preference Table

| Enzyme Encoding | Carbohydrate Energy Source |
|---|---|
| Enzyme 1 | Carbohydrate 1 |
| Enzyme 2 | Carbohydrate 2 |
| Enzyme 3 | Carbohydrate 3 |
| Enzyme 4 | Carbohydrate 4 |
| Enzyme 5 | Carbohydrate 5 |
| Enzyme 6 | Carbohydrate 6 |
| Enzyme 7 | Carbohydrate 7 |

### Bacterium B Carbohydrate Consumption Preference Table

| Enzyme Encoding | Carbohydrate Energy Source |
|---|---|
| Enzyme 3 | Carbohydrate 3 |
| Enzyme 1 | Carbohydrate 1 |
| Enzyme 2 | Carbohydrate 2 |

### Bacterium C Carbohydrate Consumption Preference Table

| Enzyme Encoding | Carbohydrate Energy Source |
|---|---|
| Enzyme 5 | Carbohydrate 5 |
| Enzyme 6 | Carbohydrate 6 |

### Bacterium D Carbohydrate Consumption Preference Table

| Enzyme Encoding | Carbohydrate Energy Source |
|---|---|
| Enzyme 7 | Carbohydrate 7 |

### Desire to Increase Activity of Bacterium C and Bacterium D

The carbohydrate mix would include Carbohydrates 5 and 7 for the targeted bacteria as well as 3 and 1. In this case, although Bacterium A is capable of consuming any of Carbs 1-7, it prefers 1, B prefers 3, etc.

### Gut Microorganisms

Some strains of microorganisms that are present in mammalian and/or avian intestines can produce beneficial nutrients and other agents that can improve the health of the mammalian and/or avian host. Such microbially-produced beneficial nutrients and agents include bacteriocins, vitamins, short chain fatty acids (SCFAs), anti-cancer agents, neuromodulators, co-factors (e.g., NAD, CAMP, etc.), and combinations thereof. Examples of microorganisms that can populate the mammalian and/or avian gut include Lactobacillus acidophilus, Lactobacillus bulgaricus, Lactococcus lactis (subsp. lactis/creamoris), Lactobacillus delbrueckii, Lactobacillus fermentum, Lactobacillus gasseri, Lactobacillus helveticus, Lactobacillus johnsonii, Lactobacillus plantarum, Lactobacillus paracasei, Lactobacillus reuteri, Lactobacillus rhamnosus, Lactobacillus sakei, Lactobacillus salivarius, Leuconostoc gelidum, Leuconostoc pseudomesenteroides, Leuconostoc carnosum, Arthrobacter nicotinae, Bacteroides thetaiotaomicron, Bifidobacterium adolescentis, Bifidobacterium bifidum, Bifidobacterium dentium, Bifidobacterium infantis, Bifidobacterium longum, Bacillus cereus, Bacillus coagulans, Bacillus megaterium, Bacillus subtilis, Bacillus subtilis ATCC6633, Brochontrix thermosphacta, Clostridium butylicum, Clostridium acetobutylicum, Clostridium thermoaceticum, Escherichia coli strain G3/10, Escherichia coli strain G1/2, Escherichia coli strain G4/9, Escherichia coli strain G5, Escherichia coli strain G6/7, Escherichia coli strain G8, Eubacterium rectale, Eubacterium eligens, Klebsiella pneumoniae, Mycobacterium smegmatis, Nocardia restricta, Nocardia rugosa, Nocardia salmonicida, Pediococcus acidilactici, Pediococcus pentosaceus FBB61, Propionibacterium acidipropionici, Propionibacterium freudenreichii, Propionibacterium jensenii, Propionibacterium thoenii, Staphylococcus epidermidis, Staphylococcus equorum, Staphylococcus gallinarum, Staphylococcus nepalensis, Streptococcus salivarius, Staphylococcus succinus, Staphylococcus xylosus, Streptococcus hyointestinalis DPC6484, Streptococcus uberis strain 42, and Streptococcus thermophilus. While many of these bacterial species and strains thereof can act as beneficial probiotic microorganisms, some can have negative health effects. Many of the negative health effects are the result of a host diet that lacks the diversity and/or sufficient quantities of prebiotic carbohydrates to provide energy sources for the diversity of microorganisms in the gut. When gut microorganisms do not have access to the types of carbohydrates they can thrive on, the microorganism can turn to digestion of gut wall components, including mucins and intestinal wall coatings that would protect the intestine from erosion, irritation, inflammation, and disease. Moreover, the response of a mammalian and/or avian subject to various intestinal microorganisms can be affected by the health (or lack of health) of a mammalian and/or avian subject, as well as by the ratios of the amounts of the different types of microorganisms in the gut. Hence, testing to evaluate the types and relative abundance of each type of microorganism present in a subject's fecal, stomach, oral or other gastrointestinal sample facilitates the design of optimized prebiotic compositions with the objective of gently and effectively rebalancing the gut microbiota in the subject and improving the health of such a subject.

Some types of microorganisms that can be found in the mammalian or avian gut and that can have negative effects on the health of the mammalian or avian subject include but are not limited to Clostridium difficile, Clostridium perfringens, Listeria monocytogenes, Leuconostoc pseudomesenteroides (urinary tract infections), enterohemorrhagic E. coli (EHEC), enteroinvasive E. coli (EIEC), Pseudomonas aeruginosa, Salmonella enterica, Salmonella typhi, Salmonella paratyphi A, Salmonella schottmuelleri, Salmonella hirschfeldii, Streptococcus bovis, Yersinia enterocolitica, and combinations thereof. Again, in some cases, the amounts or ratios of these microorganisms influences whether or not a health problem arises.

The following describes some of the beneficial agents that can be produced by microorganisms, and the types of microorganisms that produce them.

### Bacteriocins

Bacteriocins are peptides or small proteins, or ternary structures thereof, which exhibit antimicrobial properties. The first bacteriocin was discovered by Gratia in 1925 while studying E. coli. The bacteriocin produced by E. coli was dubbed “colicin.” This naming convention became canon, and so hence others are similarly named, e.g. B. subtilis=subtilin, P. acidilactici=pediocin, and so on.

There are four classes of bacteriocins, which are further subdivided by their molecular definition, their properties, or their activities.
Class I: lantibiotics characterized by the presence of unusual amino acids such as lanthionine or β-methyllanthione. Such unusual amino acids can arise from post-translational modification of the precursor peptide. They are thermostable and resistant to acidic pH.
Class II: Thermostable non-lantibiotic peptides.
   IIa: Single peptide bacteriocins, for example, exemplified by the pediocins which all exhibit a conserved N-terminal consensus sequence of YGNGV (SEQ ID NO: 1; Papagianni and Anastasiadou, 2009).
   IIb: two-peptide bacteriocins, for example, exemplified by lactoccin G/3147, et al. (Lactococcus lactis subsp lactis; McAuliffe, et al. 1998) and plantaricin E/423/ZJ5/NC8/W. et al. (Lactobacillus plantarum, Maldonado, et al. 2004; plantaricin W, and others, are actually a two-peptide lantibiotics and should be re-classified as Class Ib, which isn't yet codified, Holo, et al. 2001).
   IIc: see (signal peptide)-dependent secreted bacteriocins, of which enterocin P, from Lactococcus lactis, is a member (Herranz and Driessen, 2005).
Class III: Heat labile proteins exemplified by helveticin J and caseicin 80.
Class IV: Complex lipoproteins/glycoproteins.

One example of a highly useful anti-bacterial and anti-cancer bacteriocin is nisin. Nisin is a lantibiotic bacteriocin produced by Lactococcus lactis (creamoris, subsp. lactis, et al.). It was discovered in 1930, and has been marketed as a food-safe preservative (designated E234) under the trade-name “Nisaplin” by Beaminster (now DuPont) since the 1950s (Jones et al., 2005). Nisin was named for its activity, “Group N Streptococci Inhibitory Substance,” (Zorn and Czermak, 2014). As the lantiobiotic classification implies, nisin is a 34-residue peptide (3,353 Da) that contains the unusual lanthionine, methyllanthionine, didehydroalanine, and didehydroaminobutyric acid residues. Unlike many other bacteriocins, which are remarkably specific with respect to the organisms they attenuate (Reeves, 1979), nisin demonstrates broad spectrum activity at very low doses, for example, in the parts per billion (ppb) range. It is thermally stable. It is also most soluble and stable at acidic pH. Hence, it can be autoclaved at 115° C. and/or subjected to pH 2 conditions for extended periods of time.

Nisin is active against Bacillus, Clostridium, Staphylococcus, Streptococcus, and Listeria spp. (Fox, et al. 2000). Nisin has also been found effective against gram negative organisms, e.g. Salmonella spp. especially when co-administered with EDTA to prevent binding of the molecules to the peptidoglycan outer coat of the pathogenic species (Stevens, et al. 1991).

Recently, nisin has been demonstrated to reduce tumorigenesis (head and neck) in mice (Kamarajan, et al. 2015-mouse study; Shin, et al. 2015). A sequence for Nisin: ITSISLCTPGCKTGALMGCNMKTATCNCSIHVSK (SEQ ID NO:2).

Researchers have found that nisin ZP, a class I lantibiotic bacteriocin, can reduce tumorigenesis in mice (Kamarajan, et al. 2015 PLOS One. 10(7): 20 pp), presumably via CHAC1 (Joo, et al. 2012 Cancer Medicine 1(3), pp. 295-305), and that other bacteriocins have been noted to have activity against a variety of cancer cell lines (Kaur and Kaur, 2015). Hence, increasing the population of the parent organism(s) and/or increasing the metabolic activity of such organisms can increase the quantity of bacteriocin(s) that can exert a positive effect on cancerous/pre-cancerous lesions in the colon (and elsewhere).

TABLE 1
Examples of cancer types affected by bacteriocins

| | | | Bacter- | |
|---|---|---|---|---|
| | Organism | | iocin | |
| | Producing | | Size | |
| Bacteriocin | Bacteriocin | Class | (kDa) | Cancer Cell Type |
| Colicin E3 | E. coli | III | 9.8 | P388 (lymphoma), HeLa |
| | | | | (cervical cancer), HS913T |
| | | | | (fibrosarcoma) |
| Colicin A | E. coli | III | >20 | HS913T (fibrosarcoma), |
| | | | | SKUT-1 (uterine |
| | | | | leiomyosarcoma), BT474 |
| | | | | (breast cancer), ZR75 |
| | | | | (breast cancer), SKBR3 |
| | | | | (breast cancer), MRCS |
| Colicin E1 | E. coli | III | 57 | MCF7 (breast cancer), |
| | | | | HS913T (fibrosarcoma) |
| Microcin | K. pneumoniae | IIa | 7.9 | HeLa (cervical cancer), |
| E492 | | | | Jurkat (leukemia), RJ2.25 |
| | | | | (B cell line) |
| Pediocin | P. acidilactici | IIa | 3.5 | A-549 (lung cancer, |
| PA-1 | PAC1.0 | | | adenocarcinomic human |
| | | | | alveolar basal epithelial |
| | | | | cells), DLD-1 (colon |
| | | | | cancer) |
| Pediocin | P. acidilactici | IIa | 4.6 | HT29 (human colon |
| K2a2-3 | K2a2-3 | | | adenocarcinoma), HeLa |
| | | | | (human cervical |
| | | | | carcinoma |
| Pediocin | P. acidilactici | IIa | | HeLa (cervical cancer), |
| CP2 | | | | MCF7 (breast cancer), |
| | | | | HepG2 (liver cancer) |
| Pyocin S2 | P. aeruginosa | III | 74 | HepG2 (liver cancer), Im9 |
| | 42A | | | (myeloma), HeLa (human |
| | | | | cervical carcinoma), |
| | | | | AS-II (ovarian |
| | | | | carcinoma), mKS-A TU-7 |
| | | | | (transformed kidney cells) |
| Nisin | L. lactis | I | 3.5 | MCF7 (breast cancer), |
| | | | | HepG2 (liver cancer) |
| Bovicin | S. bovis HC5 | I | 2.4 | MCF7 (breast cancer), |
| HC5 | | | | HepG2 (liver cancer) |
| Smeg- | M. smegmatis | III | 75 | HeLa (cervical cancer), |
| matocin | 14468 | | | HGC-27 (gastric cancer), |
| | | | | mKS-A TU-7 |
| | | | | (transformed kidney cells) |
| Plantaricin | L. plantarum | II | 2.4 | Jurkat (leukemia), GH₄ |
| A | C11 | | | (pituitary cancer), Reh |
| | | | | (leukemia), Jurkat |
| | | | | (leukemia), PC12 (adrenal |
| | | | | chromaffin tumor), N2A |
| | | | | (spinal cord tumor), GH₄ |
| | | | | (pituitary cancer) |

Many types of bacteriocins are produced that can inhibit growth or other functions of a variety of organisms. For example, Lactobacillus salivarius (e.g., strain NRRL B-30514) can inhibit the growth of Campylobacter spp., such as Campylobacter jejuni, E. coli, and Salmonella spp. Such species of Campylobacter can colonize the gastrointestinal systems of poultry and cause deleterious effects during production and processing of poultry.

Other examples of bacterial species that can produce bacteriocins are provided in Table 2.

TABLE 2
Bacterial species that produce bacteriocins with broad spectrum activity

| Bacteriocin | Bacteria | References |
|---|---|---|
| Microcin S, E492 | Escherichia coli G3/10; | Zschuttig, et al. 2012; |
| | Klebsiella pneumoniae | Hetz, et al. 2002. |
| Pediocin A; PA-1 | Pediococcus | Piva, et al. 1994; Simha, |
| | pentosaceus | et al. 2012; Chikindas, et |
| | FBB61, NCDC 273; | al. 1993; Rodriguez, et al. |
| | Pediococcus | 2002 |
| | acetolactici | |
| | PAC1.0 | |
| Nisin A; Z; ZP; H; | Lactococcus lactis | Lianou and Samelis, |
| U | subsp. cremoris M104; | 2014; Delves-Broughton, |
| | Lactococcus lactis | 1990; de Vos, et al. 1993; |
| | subsp. lactis | Shin, et al. 2016; |
| | NIZO 22186; | O'Connor, et al. 2015; |
| | Streptococcus | Wirawan, et al. 2006 |
| | hyointestinalis | |
| | DPC6484; | |
| | Streptococcus uberis | |
| | strain 42 | |
| Subtilin | Bacillus subtilis | Entian and de Vos, 1996 |
| | ATCC6633 | |
| Plantaricin A; EF; | Lactobacillus | Diep et al, 1994; |
| JK; S; W; ZJ5; | plantarum C11, | Maldonado, et al. 2004; |
| ST31; K25 | NC8, ZJ5; ST31 | Holo, et al. 2001; Song, |
| | | et al. 2014; Todorev et al, |
| | | 2004; Lim et al. 2016; |
| | | Zacharof and Lovitt, |
| | | 2012 |
| Sakacin A; G; P; | Lactobacillus sakei | Holck, et al. 1992; Simon |
| C2 | Lb706; 2512; | et al. 2002; Mathiesen et |
| | Lb674; C2 | al. 2005; Gao et al. 2011 |
| Lactacin B; F | Lactobacillus | Barefoot and |
| | acidophilus N2; | Klaenhammer, 1983; |
| | Lactobacillus | Zacharof and Lovitt, |
| | johnsonii spp.; | 2012; Muriana and |
| | Lactobacillus | Klaenhammer, 1991; |
| | acidophilus 11088 | Tabasco et al, 2009 |
| | (NCK88); | |
| | Lactobacillus | |
| | acidophilus La-5 | |
| Lactocin 27; 160; | Lactobacillus | Upreti and Hinsdill, |
| 705; S | helveticus LP27; | 1975; Turovskiy, et al. |
| | Lactobacillus | 2009; Zacharof and |
| | rhamnosus 160; | Lovitt, 2012; Castellano, |
| | Lactobacillus | et al. 2003; Mortvedt- |
| | casei CRL705; | Abildgaard, et al. 1995. |
| | Lactobacillus sake L45 | |
| Lactoccin G | Lactococcus lactis spp. | Zacharof and Lovitt, |
| | | 2012 |
| Lactococcin MN | Lactococcus lactis | Zacharof and Lovitt, |
| | subsp. cremoris | 2012 |
| Leucosin A; A- | Leuconostoc gelidum | Hastings, et al. 1991; |
| UAL 187; B- | UAL 187; | Zacharof and Lovitt, |
| KM432Bz; H; B- | Leuconostoc | 2012; Makhloufi et al. |
| Ta11a | pseudomesenteroides | 2013; Felix, et al. 1994 |
| | KM432Bz; | |
| | Leuconostoc | |
| | carnosum Ta11a | |
| Entianin | Bacillus subtilis | Fuchs, et al. 2011 |
| | subsp. spizizenii | |
| | DSM 15029 | |
| Gassericin | Lactobacillus gasseri | |
| Bacteriocin PJ4 | Lactobacillus | Jena, et al. 2013 |
| | helveticus PJ4 | |
| Unnamed | Lactobacillus | Tufail, et al. 2011; |
| | delbrueckii subsp. | Michaylova, et al. 2007 |
| | Bulgaricus GLB44 | |
| Epidermin | Staphylococcus | Bierbaum, et al. 1996; |
| | epidermidis Tu | Gotz, et al. 2014; Bonelli |
| | 3298 | et al, 2006 |
| Gallidermin | Staphylococcus | Bierbaum, et al. 1996; |
| | gallinarum Tu | Gotz, et al. 2014; Bonelli |
| | 3928 (F16/P57) | et al, 2006; Kellner, et al. |
| | | 1988 |
| Epilancin K7; 15X | Staphylococcus | Bierbaum, et al. 1996; |
| | epidermis 15X154 | Velasquez et al, 2011 |
| Mersacidin | Bacillus cereus T; | Brotz, et al. 1997; |
| | Bacillus sp. strain | Altena, et al. 2000 |
| | HIL Y-85,54728 | |
| Bacteriocin | Lactobacillus | Riboulet-Bisson et al., |
| Abp118 | salivarius (e.g., | 2012 |
| | strain NRRL | |
| | B-30514) | |

### Vitamins

Although perhaps 39% of the western population is deficient in some types of vitamins, the vegan diet can be particularly troublesome (Pawlak, et al. 2013) because some nutrients required for complete nutrition are typically sourced from meat, for example, cyanocobalamin or vitamin B12. Because meat is expensive and is avoided by vegan/vegetarians, acceptable alternative sources would be beneficial. For example, Table 3 lists bacterial species that may be present in the gut, that may biosynthesize dietary vitamins, and that can be selected as a ‘target’ to be fostered by the prebiotic compositions described herein.

TABLE 3
Bacteria that Biosynthesize Vitamins

| Vitamin | Bacteria | References |
|---|---|---|
| Riboflavin, | Bacillus subtilis, | Lim, et al. 2001; |
| vitamin B2 | Clostridium butylicum | LeBlanc, et al. 2013; |
| | Clostridium acetobutylicum, | Perkins and Pero, |
| | | 2002; |
| Cobalamin, | Propionibacterium | LeBlanc, et al. 2013; |
| vitamin B12 | freudenreichii*, | Martens, et al. 2002; |
| | Lactobacillus reuteri CRL | Taranto, et al. 2003; |
| | 1098, | Hollreigl, et al. 1982 |
| | Bacillus megaterium ATCC | |
| | 13693, | |
| | Nocardia rugosa DSM43194, | |
| | Clostridium thermoaceticum | |
| Niacin/thiamin/ | Streptococcus thermophilus | LeBlanc, et al. 2013; |
| pyridoxine, | ST5**, | B3/B6. Alm, 1982; |
| vitamins B3, | Lactobacillus helveticus | Shahani and |
| Bl, and B6 | R0052, Bifidobacterium | Chandan, 1979; |
| | longum R0175 | B1/B6: Champagne, |
| | | et al. 2010 |
| Folate, | Lactobacillus plantarum | Rossi, et al. 2011; |
| vitamin B9 | JDM1, WCFS1; | LeBlanc, et al. 2007. |
| | Bifidobacterium adolescentis | |
| | ATCC 15703; | |
| | Bifidobacterium dentium Bd1; | |
| | Streptococcus thermophilus; | |
| | Lactococcus lactis subsp | |
| | lactis/creamoris; | |
| | Lactobacillus delbrueckii | |
| | subsp. bulgaricus; | |
| | Propionibacterium thoenii; | |
| | Propionibacterium | |
| | acidipropionici; | |
| | Propionibacterium jensenii; | |
| | Bifidobacterium | |
| | pseudocatenulatum; | |
| | Leuconostoc lactis; | |
| | Leuconostoc | |
| | paramesenteroides | |
| Menaquinone | Lactococcus lactis subsp | Walter, et al. 2013 |
| series, | lactis/creamoris; | |
| vitamin K1-11 | Leuconostoc Lochs; | |
| | Brochontrix thermosphacta; | |
| | Staphylococcus xylosus; | |
| | Staphylococcus equorum; | |
| | Bacillus subtilis; | |
| | Arthrobacter nicotinae | |

*Dependent on co-fermentation with L. helveticus which provides essential amino acids (McCarthy, 2004), and has been noted to possibly lower incidence of colon cancer (in rats, Lan, 2008) ostensibly via action of SCFA on cellular mitochondria (Jan, 2002).
**Commonly associated and synergistic with L. delbrueckii subsp. bulgaricus which is a proteolytic organism (Courtin and Rul, 2003), of which several strains are known to produce bacteriocins (GLB44, BB18, Simova, et al. 2008), and at least one strain, VSL3, is useful (in combination with conventional treatment) in treatment of ulcerative colitis (Ghouri, et al. 2014) via reduction in colonic inflammation (see also L. fermentum, Hegazy and El. Bedewy, 2010).

### Antibiotics

Microorganisms are the original source of many anti-microbial compounds. For example, Bacillus brevis makes gramicidin, which is one of the first antibiotics to be manufactured commercially. It is a heterogeneous mixture of six antibiotic compounds, all of which are obtained from the soil bacterial species Bacillus brevis.

Lactobacillus salivarius (e.g., strain NRRL B-30514) can produce bacteriocin Abp118 (also called UCC118), and can inhibit the growth of Campylobacter spp., such as Campylobacter jejuni, E. coli, and Salmonella spp. See, Riboulet-Bisson et al., Effect of Lactobacillus salivarius Bacteriocin Abp118 on the Mouse and Pig Intestinal Microbiota, PLOS One 7(2): e31113 (2012); Stern et al., Antimicrob Agents Chemother 50(9): 3111-16 (2006).

Streptomyces is the largest antibiotic-producing genus, producing antibacterial, antifungal, and antiparasitic drugs, as well as a wide range of other bioactive compounds, such as immunosuppressants. Streptomyces species produce over two-thirds of the clinically useful antibiotics of natural origin. For example, members of the Streptomyces genus are the source for numerous antibacterial agents such as Chloramphenicol (from S. venezuelae), Lincomycin (from S. lincolnensis), Neomycin (from S. fradiae), and Tetracycline (from S. rimosus and S. aureofaciens). Further, S. mediterranei (renamed Amycolatopsis rifamycinica in 2004) was found to produce rifamycin, a broad spectrum antibiotic with few cross tolerances. It is effective against HIV-related tuberculosis, and for the treatment of traveler's diarrhea. An orally active form of rifampicin may reduce the number of advanced glycation end products (AGEs). Incubation with rifamycin can also increase the lifespan of Caenorhabditis elegans by up to 60% (Golegaonkar, et al. Aging Cell 14(3): 463-73 (2015). Rifamycin has further been noted to be cytotoxic to ascites tumors (Hughes et al., Oncology 35(2): 76-82 (1978); Hughes & Calvin, Cancer Lett, August 1978), see webpage at digital.library.unt.edu/ark:/67531/metadc833842/m2/1/high_res_d/1014065.pdf). In addition, some Pseudomonas spp. may produce compounds antagonistic to other soil microbes, such as phenazine-type antibiotics or hydrogen cyanide.

### Short Chain Fatty Acids

Short-chain fatty acids (SCFAs) are one of the metabolites produced in the gut through fermentation of dietary fibers by the anaerobic intestinal microbiota. Such SCFAs have been shown to exert multiple beneficial effects on mammalian and/or avian energy metabolism. Three phyla of microorganisms: the Bacteroidetes (gram-negative), the Firmicutes (gram-positive), and the Actinobacteria (gram-positive) are the most abundant in the intestine. The Bacteroidetes phylum mainly produces acetate and propionate, whereas the Firmicutes phylum has butyrate as its primary metabolic end product.

Different intestinal microorganisms exert a strong impact on energy storage and interact with the host lipoprotein lipase (LPL)-mediated process for triglyceride storage in adipocytes. For example, microorganisms can suppress the intestinal epithelium expression of the LPL-inhibitor fasting-induced adipose factor, while promoting the absorption of polysaccharides from the gut lumen (Backhed et al., 2004). Intestinal microorganisms can also increase glucose uptake in the host intestine and produce a substantial elevation in serum glucose and insulin, stimulating the hepatic lipogenesis.

However, such effects can be modulated by fostering the growth of the types of microorganisms that produce small chain fatty acids such as acetate, propionate, and butyrate as their primary metabolic end products. Gut microbiota can enhance energy yields of what would otherwise be indigestible fibers by processing the indigestible dietary polysaccharides to SCFAs. For example, the SCFAs can constitute a fundamental energy source for human colonic epithelium by providing from 5 to 15% of total energy requirements. Approximately 30% of dietary calories are made for the host by the microbial species in the gut. In addition, SCFAs can signal upregulation of serotonin via interaction with the vagus nerve, and may alleviate mood disorders. Some of the benefits of microbial SCFA production in the colon are described in Table 4.

TABLE 4
Health Benefits of Short Chain Fatty Acids in the Colon

| SCFA | Specific Effect | Benefit |
|---|---|---|
| Total | Lowering of pH | Diminished bioavailability of |
| SCFA | | alkaline cytotoxic compounds. |
| | | Inhibition of growth of pH |
| | | sensitive organisms. |
| Acetate | Possible increase in Ca | Diminished fecal loss of |
| | and Mg absorption. | Ca and Mg. |
| | Relaxation of resistance | Greater colonic and hepatic |
| | vessels. | portal venous blood flow. |
| Propionate | Enhanced colonic | Easier laxation, relief of |
| | muscular contraction. | constipation. |
| | Relaxation of resistance | Greater colonic and hepatic |
| | vessels. | portal venous blood flow. |
| | Stimulation of colonic | Greater ion and fluid absorption, |
| | electrolyte transport. | prevention of diarrhea. |
| | Colonic epithelial | Greater absorptive |
| | proliferation. | capacity. |
| Butyrate | Relaxation of | Greater colonic and hepatic |
| | resistance vessels. | portal venous blood flow. |
| | Metabolism by | Maintenance of mucosal |
| | colonocytes. | integrity, repair of diversion |
| | Maintenance of normal | and ulcerative colitis, |
| | colonocyte phenotype. | colonocyte proliferation. |
| | Stimulation of colonic | Diminished risk of malignancy. |
| | electrolyte transport | Greater ion and fluid absorption, |
| | | prevention of diarrhea. |

Studies of human fecal microbial communities indicate that at pH 5.5 the butyrate-producing bacteria such as Roseburia spp., Clostridium acetylbutylicum, Clostridium butylicum, Clostridium beijerinkii, and Faecalibacterium prausnitzii, all belonging to the Firmicutes phylum, can make up a substantial percentage (e.g., 20% or more) of the total population of intestinal microorganisms. However, when fermentable dietary fibers become limiting in the more distal parts of the large intestine, the luminal pH can increase to 6.5, the butyrate-producing bacteria almost completely disappear, and the acetate and propionate-producing Bacteroides-related bacteria can become dominant. Studies of the interaction between two members of the two major divisions of the human intestinal microbiota: the Bacteroidetes (e.g., B. thetaiotaomicron) and the Firmicutes (e.g., E. rectale) illustrate the syntrophic relationship and interplay between these microorganisms (see, e.g., Mahowald et al., 2009). Syntrophism has been characterized by comparing the whole genome transcriptional profiling of each species in monoassociated and biassociated gnotobiotic mice. According to Mahowald et al. (2009), B. thetaiotaomicron adapts to the presence of E. rectale by the upregulation of polysaccharide utilization loci that confer to the microorganism the capacity to increase the variety of glycan substrates utilized, including those derived from the host and that E. rectale is unable to access. E. rectale can respond to B. thetaiotaomicron with down-regulation of glycoside hydrolases and the up-regulation of three simple sugar transport systems for cellobiose, galactoside and arabinose/lactose, as well as peptide and amino acid transporters. Moreover, the E. rectale enzymes involved in the production of butyrate are the most highly expressed in mice having both types of microorganisms in their guts. Taken together, these observations indicate that E. rectale is better able to access nutrients in the presence of B. thetaiotaomicron and utilizes the B. thetaiotaomicron-derived acetate to generate increasing amounts of butyrate in mouse colon.

In response to a standard polysaccharide-rich chow diet β. thetaiotaomicron upregulates several polysaccharide utilization genetic loci involved in the degradation of dietary plant polysaccharides (e.g., soluble hemicelluloses and some celluloses) and E. rectale responds with the concomitant upregulation of sugar transporters and glycoside hydrolases. The final result is a balanced syntrophic metabolism where B. thetaiotaomicron processes complex plant polysaccharides and distributes the products of digestion to E. rectale, which in turn synthesizes butyrate. This nutrient interchange between Bacteroidetes and Firmicutes is dramatically interrupted when mice were fed with a high-fat and high-sugar Western-type diet. The highly adaptive B. thetaiotaomicron responds to this change in diet by the up-regulation of polysaccharide utilization loci specific for the degradation of the host mucus polysaccharides. Such degradation of host mucus polysaccharides can be a cause of intestinal problems for the host. Completely devoid of glycoside hydrolases that can process host glycans, E. rectale responds to the high-fat and high-sugar Western-type diet with the down-regulation of several glycoside hydrolases and sugar transporters, with an overall marked reduction in gut E. rectale colonization levels and a lower butyrate production. These data are in agreement with the observations that human subjects fed with diets deficient in complex polysaccharides harbor low levels of butyrate producer Firmicutes, such as E. rectale (Duncan et al, 2007) in their gastrointestinal tracts.

The proper balance to avoid these intestinal problems can be achieved by ingestion of the prebiotic compositions described herein that can contain complex plant polysaccharides that B. thetaiotaomicron can process to foster E. rectale synthesis of butyrate.

### SUMMARY OF EXPERIMENTAL RESULTS

The Examples described herein illustrate the following:
1. Lactococcus lactis is present at low levels and is stable in the baseline average population of microbiota in the human gut.
2. The Lactococcus lactis organism, by virtue of its genealogical encoding for the expression of DexA and DexB encoding for oligo-1,6-glucosidase and 1,6-glucosidase, is predicted to consume MIMO.
3. Lactococcus lactis was given a weighted prebiotic index (PrbI) for MIMO (IMO) of 83 (60, if looking only at match quality >98%) based on the number of hits found in the UniProt (and ancillaries) database.
4. The expression of the oligo-1,6-glucosidase and 1,6-glucosidase enzymes was confirmed via in-vitro fermentation using MIMO (ISOThrive™) as a sole carbon source by Lactococcus lactis subsp. lactis NRRL B-1821.
5. The resulting broth was demonstrated to contain at least one bacteriocin, where the activity was detected via disk-diffusion method using Weissella viridescens NRRL B-1951 as a susceptible test organism vs. standard Nisin (Sigma N5764, 2.5%) administered at 10 μg as a control).
6. The population of Lactococcus lactis in a human test group (N=13) increased by a factor of ten within 6 weeks (12 week total) of a trial where the human subjects consumed 1 g dry equivalent ISOThrive™ MIMO daily.

These results demonstrate the following
1. The methods described herein can identify which organisms are natively present in the human gut.
2. The methods described herein can identify which of the organisms detected in the gut can consume a given prebiotic (e.g., based on genes encoding the appropriate hydrolase enzymes).
3. The population of the bacteria present in the gut can be manipulated in a pre-determined way via intervention including a prebiotic.
4. The prebiotic can be selected to favor specific groups of bacteria.
5. The prebiotic may be a tailor-made cocktail of various prebiotic compositions, e.g. MIMO+FOS/GOS/XOS, etc. that is specific for the microbiome of an individual person.
6. The prebiotic composition can be specifically made and selected to foster the growth of certain organisms known to make bacteriocins.
7. Types of bacteriocins that can be produced by these methods include the class I, and/or class IIa/IIb bacteriocins.
8. These types of bacteriocins have been demonstrated to be effective at mitigating tumorigenesis and/or proliferation of pre-cancerous lesions.
9. The prebiotic treatments can, by fostering the growth of specific types of bacterial populations of the gut, increase the amount of desired bacteriocin(s) and thereby exert a positive effect on the outcome and/or progress of colon cancer of various kinds.
10. The cancer types can be targeted via susceptibility of the respective cell-line to the bacteriocin in question.
11. Cross-referencing the available databases with a-priori terms, e.g. DLD-1 (Dukes' type C, colorectal adenocarcinoma cell line) and the corresponding bacteriocin, e.g. Pediocin-A, can greatly expedite the program by minimizing the use of brute-force bioinformatics.

### Examples of 16S rRNA Sequences

Table provides some rDNA sequences for 16S ribosomal RNAs from various bacterial species. The 16S rRNA sequences can be obtained or evaluated (e.g., via BLAST) from a data base such as GreenGenes.

| Species | Sequence | |
|---|---|---|
| Lactococcus | 1 | GACGAACGCT GGCGGCGTGC CTAATACATG CAAGTTGAGC |
| lactis subsp. | 41 | GATGAAGATT GGTGCTTGCA CCAATTTGAA GAGCAGCGAA |
| cremoris | 81 | CGGGTGAGTA ACGCGTGGGG AATCTGCCTT TGAGCGGGGG |
| strain M104 | 121 | ACAACATTTG GAAACGAATG CTAATACCGC ATAACAACTT |
| 16s rRNA | 161 | TAAACATAAG TTTTAAGTTT GAAAGATGCA ATTGCATCAC |
| SEQ ID NO: 3 | 201 | TCAAAGATGA TCCCGCGTTG TATTAGCTAG TTGGTGAGGT |
| | 241 | AAAGGCTCAC CAAGGCGATG ATACATAGCC GACCTGAGAG |
| | 281 | GGTGATCGGC CACATTGGGA CTGAGACACG GCCCAAACTC |
| | 321 | CTACGGGAGG CAGCAGTAGG GAATCTTCGG CAATGGACGA |
| | 361 | AAGTCTGACC GAGCAACGCC GCGTGGGTGA AGAAGGTTTT |
| | 401 | CGGATCGTAA AACTCTGTTG GTAGAGAAGA ACGTTGGTGA |
| | 441 | GAGTGGAAAG CTCATCAAGT GACGGTAACT ACCCAGAAAG |
| | 481 | GGACGGCTAA CTACGTGCCA GCAGCCGCGG TAATACGTAG |
| | 521 | GTCCCGAGCG TTGTCCGGAT TTATTGGGCG TAAAGCGAGC |
| | 561 | GCAGGTGGTT TATTAAGTCT GGTGTAAAAG GCAGTGGCTC |
| | 601 | AACCATTGTA TGCATTGGAA ACTGGTAGAC TTGAGTGCAG |
| | 641 | GAGAGGAGAG TGGAATTCCA TGTGTAGCGG TGAAATGCGT |
| | 681 | AGATATATGG AGGAACACCG GTGGCGAAAG CGGCTCTCTG |
| | 721 | GCCTGTAACT GACACTGAGG CTCGAAAGCG TGGGGAGCAA |
| | 761 | ACAGGATTAG ATACCCTGGT AGTCCACGCC GTAAACGATG |
| | 801 | AGTGCTAGAT GTAGGGAGCT ATAAGTTCTC TGTATCGCAG |
| | 841 | CTAACGCAAT AAGCACTCCG CCTGGGGAGT ACGACCGCAA |
| | 881 | GGTTGAAACT CAAAGGAATT GACGGGGGCC CGCACAAGCG |
| | 921 | GTGGAGCATG TGGTTTAATT CGAAGCAACG CGAAGAACCT |
| | 961 | TACCAGGTCT TGACATACTC GTGCTATTCC TAGAGATAGG |
| | 1001 | AAGTTCCTTC GGGACACGGG ATACAGGTGG TGCATGGTTG |
| | 1041 | TCGTCAGCTC GTGTCGTGAG ATGTTGGGTT AAGTCCCGCA |
| | 1081 | ACGAGCGCAA CCCCTATTGT TAGTTGCCAT CATTAAGTTG |
| | 1121 | GGCACTCTAA CGAGACTGCC GGTGATAAAC CGGAGGAAGG |
| | 1161 | TGGGGATGAC GTCAAATCAT CATGCCCCTT ATGACCTGGG |
| | 1201 | CTACACACGT GCTACAATGG ATGGTACAAC GAGTCGCGAG |
| | 1241 | ACAGTGATGT TTAGCTAATC TCTTAAAACC ATTCTCAGTT |
| | 1281 | CGGATTGTAG GCTGCAACTC GCCTACATGA AGTCGGAATC |
| | 1321 | GCTAGTAATC GCGGATCAGC ACGCCGCGGT GAATACGTTC |
| | 1361 | CCGGGCCTTG TACACACCGC CCGTCACACC ACGGGAGTTG |
| | 1401 | GGAGTACCCG AAGTAGGTTG CCTAACCGCA AGGAGGGCGC |
| | 1441 | TTCCTAAGGT AAGACCGATG ACTGGGGTG |
| Lactococcus | 1 | TTTATTTGAG AGTTTGATCC TGGCTCAGGA CGAACGCTGG |
| lactis subsp. | 41 | CGGCGTGCCT AATACATGCA AGTTGAGCGA TGAAGATTGG |
| cremoris | 81 | TGCTTGCACC AATTTGAAGA GCAGCGAACG GGTGAGTAAC |
| SK11 strain | 121 | GCGTGGGGAA TCTGCCTTTG AGCGGGGGAC AACATTTGGA |
| SK11 | 161 | AACGAATGCT AATACCGCAT AACAACTTTA AACATAAGTT |
| 16S | 201 | TTAAGTTTGA AAGATGCAAT TGCATCACTC AAAGATGATC |
| ribosomal | 241 | CCGCGTTGTA TTAGCTAGTT GGTGAGGTAA AGGCTCACCA |
| RNA | 281 | AGGCGATGAT ACATAGCCGA CCTGAGAGGG TGATCGGCCA |
| SEQ ID NO: 4 | 321 | CATTGGGACT GAGACACGGC CCAAACTCCT ACGGGAGGCA |
| | 361 | GCAGTAGGGA ATCTTCGGCA ATGGACGAAA GTCTGACCGA |
| | 401 | GCAACGCCGC GTGAGTGAAG AAGGTTTTCG GATCGTAAAA |
| | 441 | CTCTGTTGGT AGAGAAGAAC GTTGGTGAGA GTGGAAAGCT |
| | 481 | CATCAAGTGA CGGTAACTAC CCAGAAAGGG ACGGCTAACT |
| | 521 | ACGTGCCAGC AGCCGCGGTA ATACGTAGGT CCCGAGCGTT |
| | 561 | GTCCGGATTT ATTGGGCGTA AAGCGAGCGC AGGTGGTTTA |
| | 601 | TTAAGTCTGG TGTAAAAGGC AGTGGCTCAA CCATTGTATG |
| | 641 | CATTGGAAAC TGGTAGACTT GAGTGCAGGA GAGGAGAGTG |
| | 681 | GAATTCCATG TGTAGCGGTG AAATGCGTAG ATATATGGAG |
| | 721 | GAACACCGGT GGCGAAAGCG GCTCTCTGGC CTGTAACTGA |
| | 761 | CACTGAGGCT CGAAAGCGTG GGGAGCAAAC AGGATTAGAT |
| | 801 | ACCCTGGTAG TCCACGCCGT AAACGATGAG TGCTAGATGT |
| | 841 | AGGGAGCTAT AAGTTCTCTG TATCGCAGCT AACGCAATAA |
| | 881 | GCACTCCGCC TGGGGAGTAC GACCGCAAGG TTGAAACTCA |
| | 921 | AAGGAATTGA CGGGGGCCCG CACAAGCGGT GGAGCATGTG |
| | 961 | GTTTAATTCG AAGCAACGCG AAGAACCTTA CCAGGTCTTG |
| | 1001 | ACATACTCGT GCTATTCCTA GAGATAGGAA GTTCCTTCGG |
| | 1041 | GACACGGGAT ACAGGTGGTG CATGGTTGTC GTCAGCTCGT |
| | 1081 | GTCGTGAGAT GTTGGGTTAA GTCCCGCAAC GAGCGCAACC |
| | 1121 | CCTATTGTTA GTTGCCATCA TTAAGTTGGG CACTCTAACG |
| | 1161 | AGACTGCCGG TGATAAACCG GAGGAAGGTG GGGATGACGT |
| | 1201 | CAAATCATCA TGCCCCTTAT GACCTGGGCT ACACACGTGC |
| | 1241 | TACAATGGAT GGTACAACGA GTCGCGAGAC AGTGATGTTT |
| | 1281 | AGCTAATCTC TTAAAACCAT TCTCAGTTCG GATTGTAGGC |
| | 1321 | TGCAACTCGC CTACATGAAG TCGGAATCGC TAGTAATCGC |
| | 1361 | GGATCAGCAC GCCGCGGTGA ATACGTTCCC GGGCCTTGTA |
| | 1401 | CACACCGCCC GTCACACCAC GGGAGTTGGG AGTACCCGAA |
| | 1441 | GTAGGTTGCC TAACCGCAAG GAGGGCGCTT CCTAAGGTAA |
| | 1481 | GACCGATGAC TGGGGTGAAG TCGTAACAAG GTAGCCGTAT |
| | 1521 | CGGAAGGTGC GGCTGGATCA CCTCCTTT |
| Bacillus | 1 | CGAACGCTGG CGGCGTGCCT AATACATGCA AGTCGAGCGG |
| subtilis | 41 | ACAGATGGGA GCTTGCTCCC TGATGTTAGC GGCGGACGGG |
| subsp. | 81 | TGAGTAACAC GTGGGTAACC TGCCTGTAAG ACTGGGATAA |
| spizizenii | 121 | CTCCGGGAAA CCGGGGCTAA TACCGGATGC TTGTTTGAAC |
| 16S | 161 | CGCATGGTTC AAACATAAAA GGTGGCTTCG GCTACCACTT |
| ribosomal | 201 | ACAGATGGAC CCGCGGCGCA TTAGCTAGTT GGTGAGGTAA |
| RNA | 241 | TGGCTCACCA AGGCAACGAT GCGTAGCCGA CCTGAGAGGG |
| SEQ ID NO: 5 | 281 | TGATCGGCCA CACTGGGACT GAGACACGGC CCAGACTCCT |
| | 321 | ACGGGAGGCA GCAGTAGGGA ATCTTCCGCA ATGGACGAAA |
| | 361 | GTCTGACGGA GCAACGCCGC GTGAGTGATG AAGGTTTTCG |
| | 401 | GATCGTAAAG CTCTGTTGTT AGGGAAGAAC AAGTACCGTT |
| | 441 | CGAATAGGGC GGTACCTTGA CGGTACCTAA CCAGAAAGCC |
| | 481 | ACGGCTAACT ACGTGCCAGC AGCCGCGGTA ATACGTAGGT |
| | 521 | GGCAAGCGTT GTCCGGAATT ATTGGGCGTA AAGGGCTCGC |
| | 561 | AGGCGGTTTC TTAAGTCTGA TGTGAAAGCC CCCGGCTCAA |
| | 601 | CCGGGGAGGG TCATTGGAAA CTGGGGAACT TGAGTGCAGA |
| | 641 | AGAGGAGAGT GGAATTCCAC GTGTAGCGGT GAAATGCGTA |
| | 681 | GAGATGTGGA GGAACACCAG TGGCGAAGGC GACTCTCTGG |
| | 721 | TCTGTAACTG ACGCTGAGGA GCGAAAGCGT GGGGAGCGAA |
| | 761 | CAGGATTAGA TACCCTGGTA GTCCACGCCG TAAACGATGA |
| | 801 | GTGCTAAGTG TTAGGGGGTT TCCGCCCCTT AGTGCTGCAG |
| | 841 | CTAACGCATT AAGCACTCCG CCTGGGGAGT ACGGTCGCAA |
| | 881 | GACTGAAACT CAAAGGAATT GACGGGGGCC CGCACAAGCG |
| | 921 | GTGGAGCATG TGGTTTAATT CGAAGCAACG CGAAGAACCT |
| | 961 | TACCAGGTCT TGACATCCTC TGACAATCCT AGAGATAGGA |
| | 1001 | CGTCCCCTTC GGGGGCAGAG TGACAGGTGG TGCATGGTTG |
| | 1041 | TCGTCAGCTC GTGTCGTGAG ATGTTGGGTT AAGTCCCGCA |
| | 1081 | ACGAGCGCAA CCCTTGATCT TAGTTGCCAG CATTCAGTTG |
| | 1121 | GGCACTCTAA GGTGACTGCC GGTGACAAAC CGGAGGAAGG |
| | 1161 | TGGGGATGAC GTCAAATCAT CATGCCCCTT ATGACCTGGG |
| | 1201 | CTACACACGT GCTACAATGG ACAGAACAAA GGGCAGCGAA |
| | 1241 | ACCGCGAGGT TAAGCCAATC CCACAAATCT GTTCTCAGTT |
| | 1281 | CGGATCGCAG TCTGCAACTC GACTGCGTGA AGCTGGAATC |
| | 1321 | GCTAGTAATC GCGGATCAGC ATGCCGCGGT GAATACGTTC |
| | 1361 | CCGGGCCTTG TACACACCGC CCGTCACACC ACGAGAGTTT |
| | 1401 | GTAACACCC |

### Subjects

As used herein, the terms “subject” or “patient” refers to any animal, such as a domesticated animal, a zoo animal, or a human. The “subject” or “patient” can be an animal like a dog, cat, bird, poultry, livestock, zoo animal, endangered species animal, or a human. Specific non-limiting examples of livestock that can be tested and/or treated as described herein include cattle, dairy cows, pigs, sheep, goats, horses, mules, donkeys, asses, buffalo, rabbits, chickens, turkeys, ducks, geese, Cornish game hens, guinea fowl, squabs, pigeons, and the like. Experimental animals can also be tested and/or treated as described herein. For example, such experimental animals can include rats, mice, guinea pigs, and any of the other animals listed above. Hence, the phrase mammal and/or avian includes any such animals.

The following Examples illustrates some of the experiments involved in the development of the invention.

### Example 1: Intestinal Growth of Microorganisms Upon Consumption of High Maltose Syrup by a Placebo Group

Thirteen human subjects in a ‘placebo’ group were given 1.5 mL of a high-maltose syrup per day for 12 weeks as a control for comparison to those receiving a prebiotic composition (see Example 2).

High maltose syrup contains a high percentage of maltose (e.g., 60%-65% or more) as well as some maltodextrins with different degrees of polymerization (DP 3-7; e.g., about 20% or less maltodextrins), and a small amount of glucose (e.g., 2-3%). An example of a high maltose syrup composition is shown below

High Maltose Syrup Composition

| | |
|---|---|
| Brix g/100g DS | 82.7 |
| glucose, %/brix | 2.21 |
| maltose, %/brix | 65.70 |
| DP 3, %/brix | 16.24 |
| DP 4, %/brix | 2.29 |
| DP 5, %/brix | 0.76 |
| DP 6, %/brix | 0.39 |
| DP 7, %/brix | 0.09 |

The terms DP 3, DP 4, DP 5, DP 6, and DP 7 in the list above refer to maltodextrins with different degrees of polymerization (DP 3-7).

The majority if not all of high maltose syrup is digested in the upper digestive tract (i.e. stomach and small intestine). Thus, this Example may illustrate a baseline microbial population of subjects who have not received a prebiotic. One (unproven) concern may be that ingestion of high amounts of maltose in this syrup may stimulate the growth of microorganisms in the upper digestive tract, and that these types of microorganisms may then populate the lower intestinal tract. This Example may thus illustrate the types of microorganisms that can be present when subjects have a high sugar (low fiber) diet. In addition, this Example describes some of the assay procedures that can be used to identify such a microbial population.

Each subject submitted three fecal swabs. The first swab was submitted at one week before taking the high maltose placebo, the second swab was submitted the day before taking the placebo and the final sample at the end of week 12 after taking the placebo.

The rRNA from the fecal samples was isolated and the diversity of 16s rRNA sequences in the samples were identified by sequencing. The sequences were cross-referenced via Basic Local Alignment Search Tool (BLAST) analysis to identify the organisms. From these data, the species with an abundance of greater than or equal to 0.1% of the total number of reads were listed.

The selected list of organisms was interrogated for enzymes that can digest oligosaccharides containing α-1,6 glycosidic linkages, e.g. oligodextran (isomaltooligosaccharides). Such enzymes are exemplified by oligo-α-1,6-glucosidase and α-1,6-glucosidase, and encoded by the DexA and DexB genes, respectively. The enzyme types were identified in the UniProt database by BLAST analysis, and the abundance of bacterial species that can ferment oligodextran was therefore predicted.

A protein BLAST (BLASTP) was performed via UNIPROTKB using the following query protein sequence (SEQ ID NO:6).

>tr|H5T0P3|H5T0P3_LACLL Oligo-1,6-glucosidase
OS = Lactocossus lactis subsp. lactis IO-1
GN = dexA PE = 4 SV = 1
MNSHLNGVVN MKENWWQKTV VYQIYPRSFM DANGDGVGDL
QGIISKLDYL EKLGIGAIWL SPVYQSPMDD NGYDISDYQA
IADVFGTMSD MDELLLEAKK RNIQIVMDLV VNHTSDEHKW
FVEARKSKDN AYRDYYIWAD EPNALQSTFS GSAWEFDEES
GQYFLHLFSK RQPDLNWENP QVHQEVYDMM NFWIDKGIGG
FRMDVIDLIG KEIDQEITGN GPKLHEYLHE MNQATFGQKN
LLTVGETWGA TPEIAELYSD PKRQELSMVF QFEHITNAYL
DEGEKWDKKE FSVSKLKEIL AKWQALEKGW NSLFWNNHDL
PRIVSNWGND GKYRLKSAKA FAILLHLMKG TPYIYQGEEL
GMTNYPFESI EEVNDIESRN MFAERLAAGH SENEIMDSIR
RVGRDNARTP MQWTAGENAG FTDGKPWLAV NPNHEEINAD
QAMSDPDSVF YTYQKLIELR KQHDWVIYGG FKLIDSEADV
FAYLRTYKGK KYLVVANLSD EENQFKTGFV CRDLLIHNEN
FLPELSQIKL KAWEAFACEV E

The run time for such a BLASTP analysis was about 2.5 minutes.

Table 5 shows a list of the alpha glucosidases, or oligo-α-1,6-glucosidases identified at the species level for L. lactis subsp. lactis which was found in the fecal samples, where the match quality is the percentage reflecting how closely the result(s) matched the protein sequence encoded by the given dexA gene. The amino acid sequence for the Lactococcus lactis glucan 1,6-alpha-glucosidase is shown above (SEQ ID NO:6). The ‘Number’ is the accession number of the gene (see website at www.ebi.ac).

TABLE 5
Glucosidases coded in L. lactis demonstrating
a match quality of >98%.

| Number | Protein Name | Identity |
|---|---|---|
| U5PLX9 | Glucan 1,6-alpha-glucosidase | 100% |
| | (Lactococcus lactis subsp. 1a) | |
| D2BKX1 | Glucan 1,6-alpha-glucosidase | 98.9% |
| | (Lactococcus lactis subsp. 1a) | |
| A0A0B8QQV6 | Glycosidases (Lactococcus lactis | 98.7% |
| | subsp. 1a) | |
| A0A089ZEV7 | Glucan 1,6-alpha-glucosidase | 98.9% |
| | (Lactococcus lactis) | |
| A0A0V8CJV8 | Glucan 1,6-alpha-glucosidase | 98.5% |
| | (Lactococcus lactis subsp. 1a) | |
| H5TOP3 | Oligo-1,6-alpha-glucosidase | 98.7% |
| | (Lactococcus lactis subsp. 1a) | |
| T0WEN3 | Glucan 1,6-alpha-glucosidase | 98.7% |
| | (Lactococcus lactis subsp. 1a) | |
| Q9CF00 | Oligo-1,6-alpha-glucosidase | 98.7% |
| | (Lactococcus lactis subsp. 1a) | |
| A0A0V8AN11 | Glucan 1,6-alpha-glucosidase | 99.4% |
| | (Lactococcus lactis subsp. 1a) | |
| S6FS83 | Trehalose-6-phosphate hydrolase | 99.1% |
| | (Lactococcus lactis subsp. 1a) | |
| A0A0V8BDL7 | Glucan 1,6-alpha-glucosidase | 99.2% |
| | (Lactococcus lactis subsp. 1a) | |
| U6EPQ0 | Glucan 1,6-alpha-glucosidase | 98.8% |
| | (Lactococcus lactis subsp. 1a) | |
| A0A0V8C313 | Glucan 1,6-alpha-glucosidase | 99.1% |
| | (Lactococcus lactis subsp. 1a) | |

Several species were noted to be natively present in low but in most of the fecal samples tested included such bacterial species. These natively present bacterial species included Bacillus subtilis, Lactococcus lactis subsp. lactis, and Pediococcus acidolactici.

Sequencing data indicated a cohort of genes were present in the fecal microorganisms that are particularly suited for glucan transport and metabolism, for example, α-1,4 and α-1,6 glucanhydrolase (glucosidase) enzymes (Table 5). These organisms are predicted to be able to utilize isomaltooligosaccharides, and specifically MIMO.

Data from this placebo group of subjects who ingested a high maltose syrup composition shows that the types of microorganisms in the gut can be detected and that a specific group of organisms that may utilize a particular ingested prebiotic composition can be identified (e.g., by identifying enzymes encoded in the genomic sequences that are present in those bacteria) in groups of mammalian and/or avian subjects.

### Example 2: Intestinal Growth of Microorganisms Upon Consumption of ISOThrive™ MIMO

Twenty-six human subjects were given 1.5 mL (1 g) of ISOThrive™ MIMO per day as a prebiotic composition for 12 weeks. Example of two batches of the ISOThrive™ composition are shown below.

ISOThrive™ Batch Compositions

| | Batch 1 | Batch 2 |
|---|---|---|
| brix: | 20.0 | 19.8 |
| mannitol | 10.26 | 24.08 |
| fructose | 0.56 | 0.08 |
| sucrose | 4.08 | 1.25 |
| maltose | 2.63 | 3.11 |
| MIMO-DP 3 | 12.04 | 8.79 |
| MIMO-DP 4 | 21.87 | 20.14 |
| 1,4-DP3 | 6.61 | 6.80 |
| MIMO-DP 5 | 13.41 | 13.96 |
| MIMO-DP 6 | 4.18 | 5.25 |
| MIMO-DP 7 | 1.63 | 2.14 |
| MIMO-DP 8 | 0.00 | 0.87 |
| 1,4-DP4 | 2.51 | 2.80 |
| MIMO-DP 9 | 0.00 | 0.00 |
| lactate | 4.23 | 10.79 |
| glycerol | 0.05 | 0.13 |
| formate | 0.00 | 0.02 |
| acetate | 1.69 | 4.10 |
| TOTAL: | 91.04 | 104.30 |
| MIMO, %: | 53.13 | 51.14 |
| Purity, %: | 58.36 | 49.03 |
| MWD: | 693.21 | 723.72 |
| Yield %: | 67.30 | 55.98 |

where 1,4-DP3, and 1,4-DP4 are maltodextrins.

Each subject submitted three fecal swabs. The first swab was submitted at one week before taking the high maltose placebo, the second swab was submitted the day before taking the placebo and the final sample at the end of week 12 after taking the placebo.

The rRNA from the fecal samples was isolated and the diversity of 16s rRNA sequences in the samples were identified by sequencing. The sequences were cross-referenced via Basic Local Alignment Search Tool (BLAST) analysis to identify the organisms. From these data, the species with an abundance of greater than or equal to 0.1% of the total number of reads were listed.

During the period of the two baseline samples, the placebo group (receiving high maltose syrup throughout, Example 1) and the intervention group (receiving ISOThrive™ MIMO, Example 2) each consumed the same high-maltose syrup placebo. During such a baseline time, an equivalent number of reads for L. lactis subsp. lactis was detected (0.048% total reads). This indicates that the subjects in the placebo and intervention groups had similar populations of microorganisms, and that the placebo (high maltose syrup) had no effect on the growth or metabolism of L. lactis subsp. lactis residing in the colon.

L. lactis subsp. lactis has a high prebiotic index for oligodextran and may utilize IMO and MIMO. In-vitro testing (2 L fermentation of 2.5% ISOThrive™ PSF at colonic physiological pH and temperature) confirmed that L. lactis subsp. lactis consumed the ISOThrive™ PSF.

In order to confirm positive growth in-vivo, the fecal microbiota of the intervention group was compared. At the conclusion of the trial, the percent of the total reads increased to 0.483% for the intervention group that received ISOThrive™ MIMO while the number of reads for the placebo group remained at 0-0.048%.

Hence, ingestion of the ISOThrive™ product, which has high levels of particular types of maltosyl-isomaltooligosaccharides (MIMOs), can facilitate growth of microorganisms in the gut that express the appropriate cohort of enzymes (glucosidase-type, in this case) required to utilize the prebiotic (MIMO, in this case).

### Example 3: Lactococcus lactis Subsp. Lactis NRRL B-1821 Growth in Media Containing Maltosyl-Isomaltooligosaccharides (MIMOs)

As indicated in Example 1, Lactococcus lactis was natively present in fecal samples of human subjects.

The number of appropriate genes encoded for the fecal sample microorganisms was sorted and weighted by quality of match (in this case, weighting factors of 5, 3, and 1 were applied for match qualities of >99, >98, and >97%, respectively. The sum of these factors is defined here as the prebiotic index for the subject organism. Thus, the higher the score, the more likely they will be able to be consumed by the subject organism. Such a prebiotic index can be a useful measure of whether a compound or composition can support the growth of a microorganism or a mixture of microorganism. The Lactococcus lactis organism was identified in the placebo group via 16S rRNA sequencing. Sequences for glucosidase-type genes (e.g., dexA and dexB, see UniProtKB website at uniprot.org/blast/uniprot/B2016062414483A1C7ED25EE8374758DF3FD545FD122F2A F) were interrogated via BLAST analysis (nucleotide to protein) to reveal 12 genes (two oligo-1,6-glucosidase, ten 1,6-glucosidase) with match quality >99%. This (weighted by match quality and number of genes) gives MIMO a potential prebiotic index (PrbI) of 60 (Q>99%, PrbI 83>97%), indicating that Lactococcus lactis are extremely likely to consume MIMO.

This Example illustrates that Lactococcus lactis strains can grow in and metabolize media containing maltosyl-isomaltooligosaccharides (MIMOs).

Lactococcus lactis subsp. lactis NRRL B-1821 was evaluated. Although not extensively studied as a probiotic organism, L. lactis subsp. lactis has a number of desirable traits such as acid-tolerance and resistance to bile (Kimoto, et al. 1999, Lett. Appl. Microbiol. 29, pp. 313-316). More recently, the species has been evaluated as a probiotic, and that certain strains exhibit anti-inflammatory potential (e.g., increased CD4+ T cells, early increases in IL-6 with sustained production of IL-10) for the treatment for inflammatory bowel disease [IBD, Luerce, et al. 2014. Gut Pathogens 6 (33), 11 pp]. Additionally, certain Lactococcus lactis strains may be capable of producing several isoforms of the lantibiotic bacteriocin nisin (Beasley and Saris, 2004 Appl. Environ Microbiol. 70 (8), pp. 5051-5053). For example, Nisin Z P (Shin et al. 2015. Front. Microbiol. 6:617), isolated from Lactococcus lactis subsp. lactis DF04Mi (Furtado, et al. 2014. Braz. J. Microbiol. 45(4), pp. 1541-1550), has recently been observed to reduce the size and proliferation of head/neck tumors in-vitro and in mice (Kamarajan, et al. 2015. PLOS One. 10(7): 20 pp).

### Methods:

M17 Media (Sigma) was prepared by dissolving 4.207 g M17 in 100.017 g (total) water (18 MΩ). The media mixture was autoclaved at 121° C. for 15 min. After cooling, the media was inoculated with Lactococcus lactis subsp. lactis NRRL B-1821 (0.5 mL late log-phase culture frozen at −78° C. in 20% glycerol), and the culture was incubated at 35° C. for 16 Hr. This culture was the inoculum for the following fermentation.

To a 2 L fermenter (New Brunswick BioFlo/Celligen 115) was added water (18 MΩ), 0.960 kg; peptone (meat), 10.080 g; yeast extract, 5.690 g; ISOThrive™ MIMO (lot #160120, a source of maltosyl-isomaltooligosaccharides (MIMOs)), 25.067 g; MnSO₄—H₂O, 0.0123 g; MgSO₄, 0.1156 g; FeSO₄·7H₂O, 0.0135 g; KH₂PO₄, 2.933 g; NaCl, 0.01208 g, and CaCl₂)-2H₂O, 0.6178 g. This fermentation media was autoclaved at 121° C. for 15 minutes. Once cooled to 35° C., a physiological temperature (36-37° C.) was maintained using a recirculating water bath. The pH of the fermentation mixture was adjusted to the physiological pH of the colon (pH 6.6) with NaOH (50% w/w) and maintained throughout at pH 6.6 using NaOH (40% w/w).

To the fermenter was aseptically added 10 mL late log-phase Lactococcus lactis subsp. lactis NRRL B-1821 inoculum, and micro-anaerobic (self-blanketing) conditions were maintained. The fermentation was allowed to proceed for 21 Hr before sampling. The headspace contained 7.79% 02 and 5.5% CO₂ (PBI Dansensor CheckMate 9900) indicating potential evolution of H₂ and/or methane.

The cells were removed via centrifugation (Sorvall RC-5B+, G3 rotor) at 13,689 g for 20 minutes. The supernatant was sampled via HPLC-RID/HPAEC-PAD and the remainder frozen at −78° C. pending analysis of bacteriocin content.

The bacterial MICs (minimum inhibitory concentration) of both nisin A and Z bacteriocins were determined via antibacterial activity vs. Weissella viridescens NRRL B-1951. A Weissella viridescens NRRL B-1951 inoculum was prepared in MRS media (5% w/w, OD>8), and incubated for 16 hr at 31° C. (to generate a late-log phase culture). Obtained from Handary S. A., and used as-is, Nisin A (95.2%) and Nisin Z (99.6%) were diluted with 18MΩ2 (Hydro Systems and Supplies, Gaithersburg, MD) water to give stock solutions containing 31 and 34 μg/g, respectively. A 5% w/w solution of MRS media (Sigma #69966) containing 0.1% Tween-80 (Sigma #P1754) was autoclaved at 121° C. for 15 minutes and kept sealed in a UV-sterilized laminar-flow hood prior to use (approximately 1 hr before testing). A set of eight samples (Hach tubes, Loveland, CO) for each nisin isoform, spanning the range of 95-57% sterile MRS media, 0-38% test broth, and nisin at 0.00-12.24 μg/g (0.00-64.73 μg/sample), was incubated with 4.5% of late-log Weissella viridescens NRRL B-1951. The absorbance at 610 nm (HACH DR900) of each sample was measured immediately after inoculation. The samples were sealed and incubated at 31° C. with orbital rotation (Thermo Forma 420) for 16 Hr. The absorbance at 610 nm of each sample was measured, the background absorbance subtracted, and the MICs were determined via plot and curve-fitting.

A small subsample of the biomass was resuspended, washed, and centrifuged again (Eppendorf 5415 C) at 12 kRPM for 15 minutes. The washed biomass pellet was re-suspended and gram stained for confirmation of culture morphology and purity via oil-immersion microscopy.

### Results

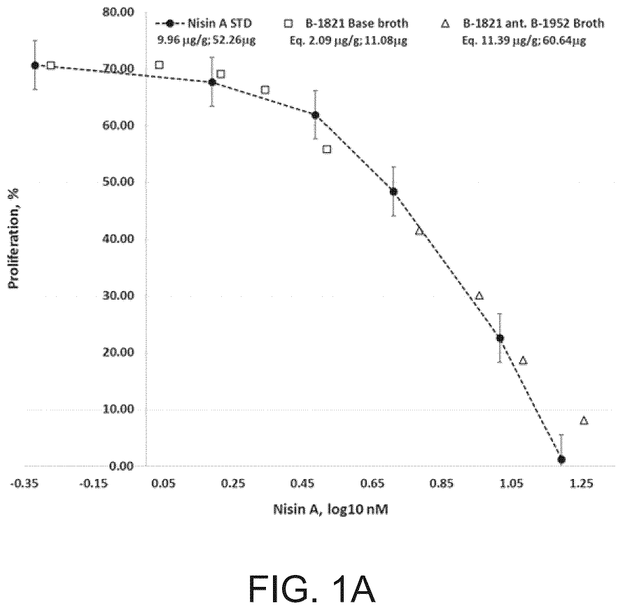

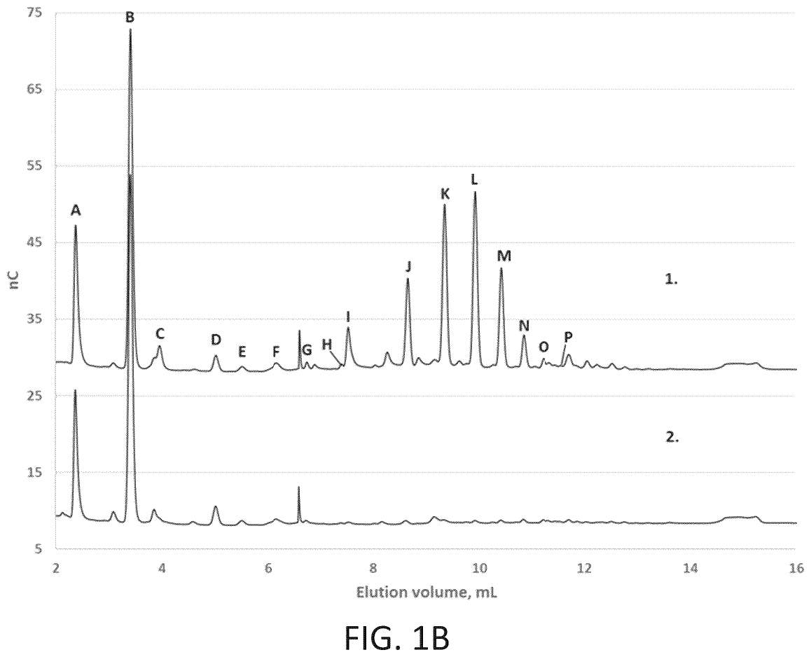

As shown in FIG. 1A, the bacteriocin nisin with sequence MSTKDFNLDL VSVSKKDSGASPRITSISLCTPGCKTGALMGCNMK TATCHCSIHVSK (SEQ ID NO: 7, coded by structural gene nisA) was detected in the culture medium by a gel diffusion assay using sodium dodecyl sulfate-polyacrylamide gel electrophoresis (SDS-PAGE), and was confirmed via tricine-PAGE vs an ultra-low molecular weight peptide standard marker (1.1-26 kDa).

Essentially all of the tested prebiotic ISOThrive™ MIMOs in the media had been consumed by 21 hours of incubation. The culture was pure and the late log morphology conformed with Lactococcus spp.

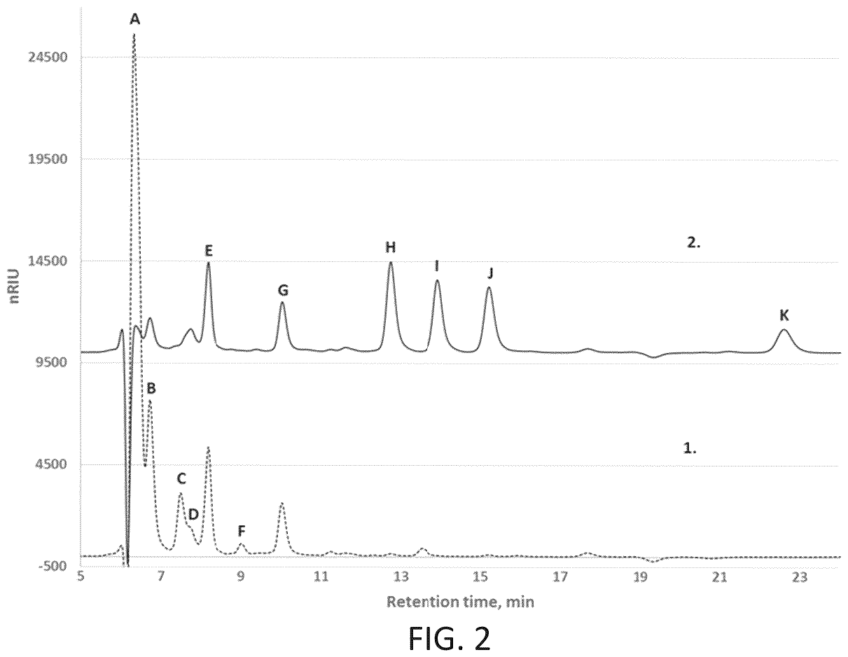

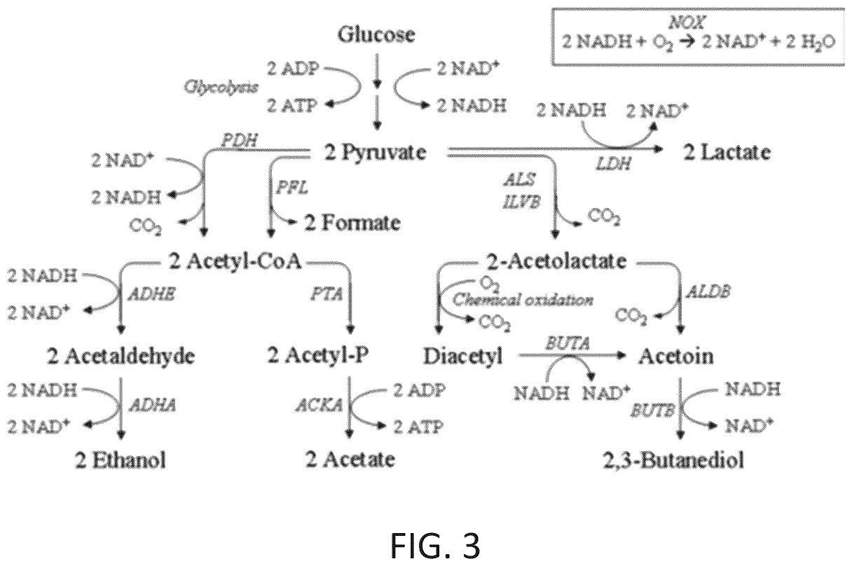

The carbohydrate profiles before and after fermentation are shown (HPAEC-PAD) for the pre-inoculum and 21 Hr samples in FIG. 1B. As illustrated in FIG. 2, pyruvate metabolism was observed when the MIMO carbon source was used during fermentation. In other words, lactate, formate, acetate, and ethanol were produced. However, 2,3-butanediol was not detected, as illustrated in FIG. 2. The pathways of fermentative metabolism for this organism are given in FIG. 3.

The broth exhibited antimicrobial activity relative to standard nisin (2.5%, Sigma-Aldrich), via disk diffusion assay vs. Weissella viridescens (susceptible organism) on MRS agar.

### Example 4: Bacillus subtilis NRRL B-23049 Growth in Media Containing Maltosyl-Isomaltooligosaccharides (MIMOs)

Microorganisms from fecal samples obtained from the human subjects who had taken 1.5 mL of a high-maltose syrup (placebo) per day, for 12 weeks, were identified via 16S rRNA sequencing, and sequences for genes from such microorganisms that may encode to glucosidase-type enzymes (e.g., dexA and dexB) were elucidated; see UniProtKB database) via BLAST analysis (nucleotide to protein) as described in Example 1.

Such analysis revealed a microorganism with two genes (one oligo-1,6-glucosidase, one 1,6-glucosidase) with match quality >99%, 3 genes >98%, and 3 genes >97%. When weighted by match quality and number of genes, the MIMO prebiotic has a potential prebiotic index of 20 indicating that this microorganism is likely to consume MIMO, but that it is not likely the preferred substrate. This organism is Bacillus subtilis.

Perhaps the oldest known probiotic, Bacillus subtilis (and many other Bacillus spp.) is able to survive by sporulation. The Bacillus spores are highly resistant to temperature and acidic pH, which explains why the species is frequently found in camel dung (which incidentally has been used directly as a probiotic to treat diarrhea by the Bedouin tribes), and why it can survive transit through the human gastrointestinal tract (Damman, et al. 2012, Am. J. Gastroenterol. 107, pp. 1452-1459). Although long regarded as an obligate aerobe, it has been found to be facultatively anaerobic (Nakano and Zuber, 1998, Ann. Rev. Microbiol. 52, pp. 165-190). These features greatly improve the probability that this microorganism can survive in the anaerobic environment of the colon (and handling to that point). Once there, and vegetative, it may be able to enhance mitogenic-induced T cell proliferation (Ciprandi et al. 1986, Chemoterapia 5 (6), pp. 404-407), and may be an effective antitumor immunotherapeutic agent [immunostimulatory effects, NK cytotoxicity augmentation, and up-regulation of IFN-a/y from leukocytes, in mice; Shlyakhovenko, et al. Experimental Oncology 25 (2), pp. 119-123]. Bacillus species have also been used as an antidiarrheal [Mazza, 1994. Boll. Chim. Farm. 133 (1), pp. 3-18].

### Methods:

Media was prepared containing 0.5% w/w each of tryptone (Sigma, casein) and yeast extract (Marcor bacteriological); 0.11% KH₂PO₄; 2.60% ISOThrive™ MIMO (lot #160120); 5.19% mannitol; and DI water (18MΩ2) to 100 mL. The media was autoclaved at 121° C. for 15 min. After cooling, the media was inoculated with Bacillus subtilis NRRL B-23049 (0.5 mL late log-phase culture frozen at −78° C. in 20% glycerol), and incubated at 35° C. for 16 Hr. This culture was the inoculum for the following fermentation.

To a 2 L fermenter (New Brunswick BioFlo/Celligen 115) was added water (18M Ω), 0.851 kg; tryptone (casein), 5.012 g; yeast extract, 5.000 g; ISOThrive™ MIMO (lot #160120), 25.100 g; KH₂PO₄, 1.018 g and mannitol, 50.000 g. This fermentation media was autoclaved at 121° C. for 15 minutes. Once cooled to physiological temperatures, 35-37° C., this temperature was maintained using a recirculating water bath. The pH was adjusted to the physiological pH of the colon (pH 6.6) with NaOH (50% w/w) and maintained throughout using NaOH (40% w/w).

To the fermenter was aseptically added 25 mL late log-phase inoculum, and micro-anaerobic (self-blanketing) conditions were maintained. The fermentation was allowed to proceed for 24 Hr before sampling. The headspace contained 8.65% O₂ and 4.4% CO₂ (PBI Dansensor CheckMate 9900). The fermenter headspace was then aerated, and the whole sampled again at 44 and 72 Hr. The cells were removed via centrifugation (Sorvall RC-5B+, G3 rotor) at 13,689 g for 20 minutes. The supernatant was sampled for analysis via HPLC-RID/HPAEC-PAD and the remainder frozen at −78° C. pending analysis of bacteriocin (target entianin, encoded by the etnS structural gene, 3.446 kDa) content, for example, by mass spectrometry. A sequence for an entianin bacteriocin is shown below (SEQ ID NO:8).

| | |
|---|---|
| 1 | MRLTISRKFS LVFLTLILIN LLVGGIGAFN MQHIIQKTDE |
| 41 | INTKWIDGIK EITSINYLTE HLSSKEKDFL IFTDKSKMDT |
| 81 | LDQEMNQILE DINQKLDSYE KTISNDKEQK LFEELQNEVN |
| 121 | TYADIHAQII ESGRTNDMDK ARGLLVQTEA SFENMKKSVT |
| 161 | QLVDFNKEGS NTAVKETKDV YHKGLIYTAS LVAASIIISI |
| 201 | FIWLYITRNI VKPIIRMKES ANHIAEGDLS SDIEPLNSKD |
| 241 | ELGDLNEALQ KMVGNLRDIV GYSKEISSRV LSSSQVLATA |
| 281 | TNETRSGSKH ITETMNEMAE GSEQQAQDAV TIAESMNEFT |
| 321 | ESIDKAYNHG ITISDTSQNV LELAVSGNEN MDTSLQQMKT |
| 361 | IHHIVQEAVH KVRSLEQHSQ DINKLVQVIN GIAEQTNLLS |
| 401 | LNAAIEAARA GESGKGFAVV AEEVRKLADG VSDSVQDITR |
| 441 | IVNGTQQEIY TVIEYLESSF TEVEKGTENL TDTGQAMQHI |
| 481 | KQSVTHVADS IKEVTDGLKQ LTNQSITINQ SIENIASVSE |
| 521 | ESAAGIEETF SITEQSAHSM DQVLQNAEEL EQLAKELNEK |
| 561 | MNQFTI |

A small subsample of the biomass was re-suspended to wash and centrifuged again (Eppendorf 5415 C) at 12 kRPM for 15 minutes. The washed biomass pellet was re-suspended and gram stained for confirmation of culture morphology and purity via gram stain/oil-immersion microscopy.

### Results

As the genetic information suggested (PrbIMIMO=20), Bacillus subtilis NRRL B-23049 was only able to partially consume MIMO.

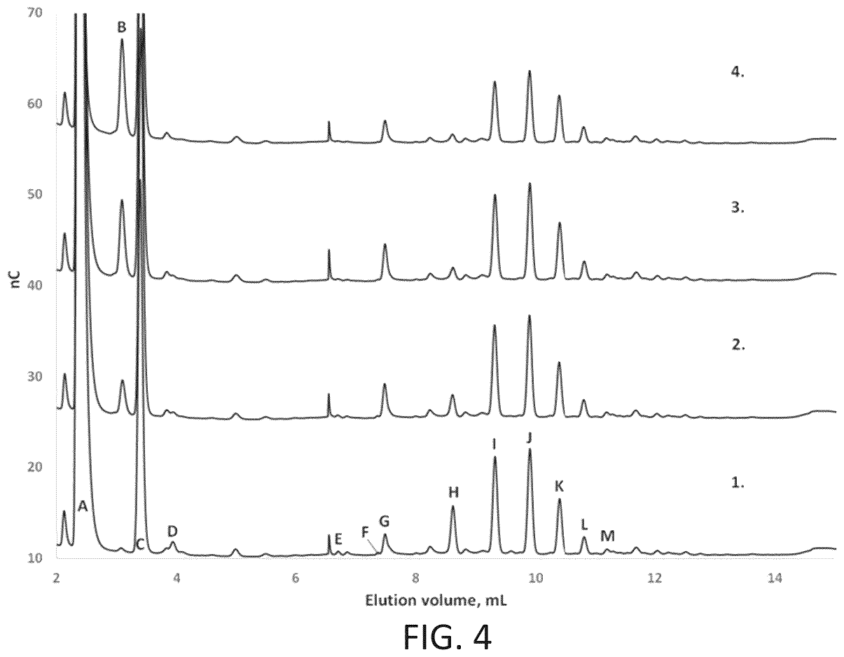

This is evident in FIGS. 4-6 where smaller carbohydrates (e.g., with lower degrees of polymerization, DP) were favored.

In particular, the carbohydrate profiles are shown (HPAEC-PAD) for the pre-inoculum and 24 Hr samples in FIG. 4. FIG. 4 illustrates overlaid HPAEC-PAD chromatograms of fermentation media containing ISOThrive™ MIMO with B. subtilis NRRL B-23049, at various time points of fermentation. Trace 1: pre-inoculum. Trace 2: media after 24 hr incubation. Trace 3 media after 44 hr fermentation. Trace 4: media after 72 hr fermentation. The components detected by HPAEC-PAD were: A, mannitol; B, unknown; C, L-arabinose (IS); D, glucose; E, isomaltotriose; F, isomaltotetraose; G, maltose, and H-M, PAN-type IMO (MIMO) DP 4-8. As illustrated, the lower degree of polymerization MIMO present in peak H disappears over time (compare pre-inoculum trace 1 with the 72 hr trace 4).

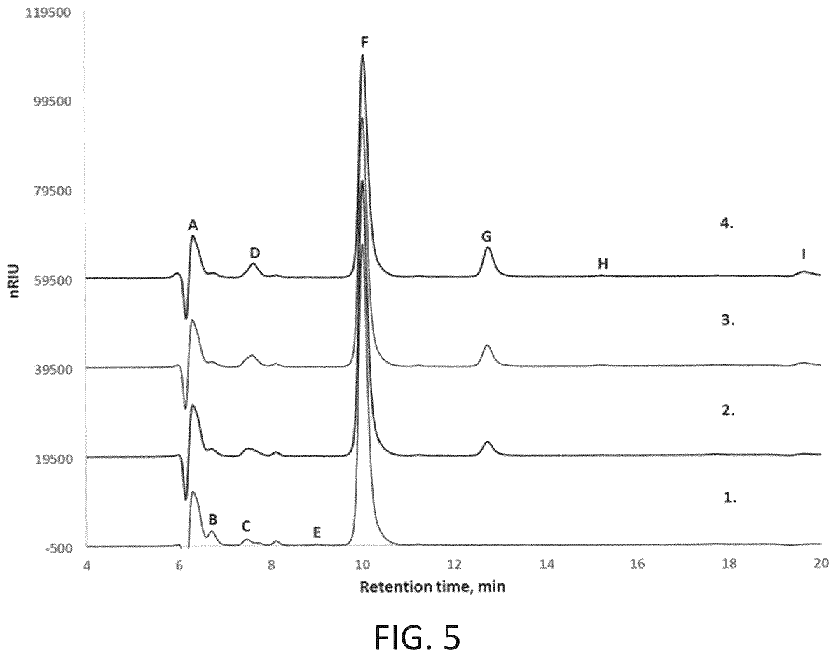

FIG. 5 illustrates the metabolic profile (HPLC-RID) of B. subtilis NRRL B-23049 during fermentation in media containing ISOThrive™ MIMO as a sole carbon source. Trace 1: Pre-inoculation media. Trace 2: media after 24 Hr fermentation. Trace 3: media after 44 Hr fermentation. Trace 4: media after 72 Hr fermentation. The components detected in the media were: A, MIMO DP >3; B, panose; C, maltose; D, leucrose; E, glucose; F, mannitol; G, lactate; H, acetate, and I, unknown diol. As illustrated, there is a decrease in MIMO (large peak at elution time 10 min) and an increase in lactate (small peak at 2.7 min elution) as time proceeds. This organism is a facultative anaerobe, but prefers the presence of oxygen. When MIMO is the carbon source, lactate and acetate metabolites were observed (e.g., no butyrate).

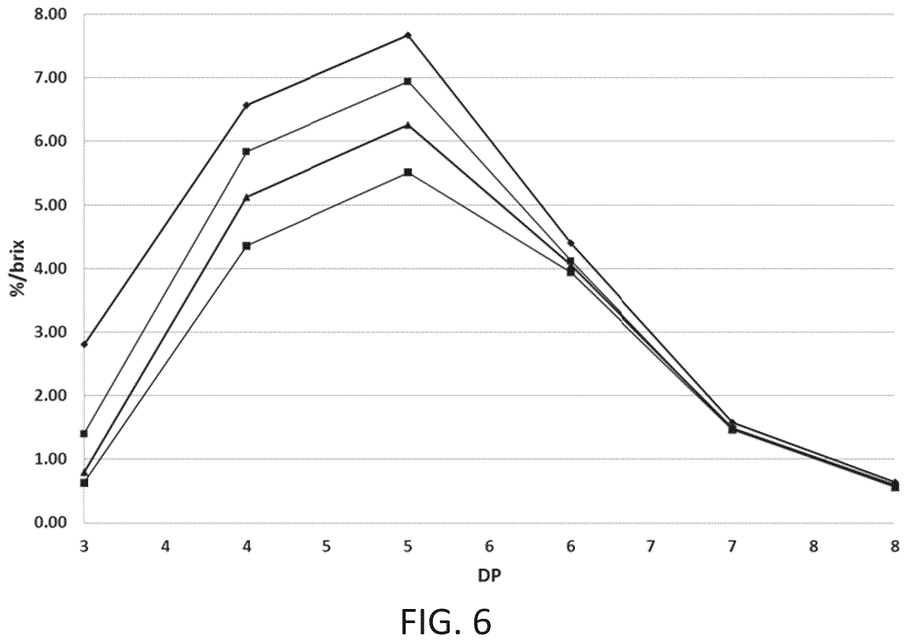

FIG. 6 graphically illustrates the rate of consumption by B. subtilis NRRL B-23049 of ISOThrive™ MIMOs with different degrees of polymerization (DP 3-7) at different time points in the fermentation. Top line: 0 hr fermentation. Second from the top line: 24 hr fermentation. Third line from the top: 44 hr fermentation. Bottom line: 72 hr fermentation. As shown in FIG. 6, MIMO was consumed at a rate of 0.103%/hr, and MIMOs with DP less than 6 were preferred substrates.

Though slow, about 50% of the tested prebiotic composition (containing ISOThrive™ MIMO (lot #160120)) was consumed by this strain of Bacillus subtilis. The culture was pure and the late log morphology conformed with Bacillus spp., indicating that the results were due to B. subtilis NRRL B-23049 metabolism, and not to any contaminants.

### Example 5: Pediococcus acidilactici NRRL B-5727 does not Grow in Media Containing Maltosyl-Isomaltooligosaccharides (MIMOs) as a Sole Carbon Source

This Example illustrates the growth and metabolism of Pediococcus acidilactici NRRL B-5727 in media containing MIMOs as a sole carbon source.

One type of microorganism was found in gut microbiota of the placebo group subjects via 16S rRNA sequencing that appeared to synthesize pediocins A, BA 28 and pre-pediocin AcH. The sequences for selected glucosidase genes of this microorganism (dexA and dexB; UniParc sequence #UPI00071AFA9B, checksum 91F60DC3EA289908, length 831, 93,587 Da) were interrogated by BLAST (nucleotide to protein) analysis. BLAST searches of these sequences provided no α-1,6-glucosidases that were coded by this microorganism with a certainty that exceeded 98%. Only 5 hits were obtained of any match quality. These data indicate that this organism may be a poor candidate for targeting by MIMO prebiotics, because this microorganism will not likely consume MIMO, and thus its growth will likely not be enhanced by IMO.

However, it should be noted that the UniRef database (see website at www.uniprot.org/uniref/UniRef50_P29430) indicates that this microorganism may synthesize pediocins A, BA 28 and pre-pediocin AcH. Sequencing data also indicated that this microorganism may metabolize fructooligosaccharides (FOS).

The probiotic (for man and animals, strain MA15/5M; Barreau et al. 2012, J. Bacteriol. 194 (4), pp. 901) organism Pediococcus acidilactici is acid stable and able to pass through the stomach intact. Pediococcus acidilactici strain NRRL B-5627 has been shown to synthesize pediocins (Guerra, et. al. 2005. Biotechnol. Appl. Biochem. 42 (1), pp. 17-23), and to produce pediocin SA-1, which is particularly effective against food borne pathogens including Listeria spp. (Anastasiodou, et al. 2008. Bioresour. Technol. 99 (13), pp. 5384-5390). Hence, the metabolism of Pediococcus acidilactici in media containing MIMOs was evaluated using the following procedures.

### Methods

MRS (Sigma) media was prepared by dissolving 5.502 g MRS in 101.387 g (total) water (18MΩ2). This media was autoclaved at 121° C. for 15 min. After cooling, the media was inoculated with Pediococcus acidilactici NRRL B-5727 (0.5 mL late log-phase culture frozen at −78° C. in 20% glycerol), and incubated at 35° C. for 16 Hr. This culture was the inoculum for the following fermentation.

To a 2 L fermenter (New Brunswick BioFlo/Celligen 115) was added water (18M Ω), 0.960 kg; peptone (meat) 10.043 g; yeast extract, 5.553 g; ISOThrive™ MIMO (lot #160120), 25.016 g; MnSO₄—H₂O, 0.01301 g; MgSO₄, 0.12952 g; FeSO₄·7H₂O, 0.01295 g; KH₂PO₄, 2.906 g; NaCl, 0.01447 g, and CaCl₂)-2H₂O, 0.06088 g. This media was autoclaved at 121° C. for 15 minutes. Once cooled to 35° C., this temperature was maintained using a recirculating water bath. The pH was adjusted to pH 6.6 with NaOH (50% w/w) and this pH was maintained throughout using NaOH (40% w/w). This culture was the inoculum for the following fermentation.

To the fermenter was aseptically added 10 mL late log-phase Pediococcus acidilactici NRRL B-5727 inoculum, and micro-anaerobic (self-blanketing) conditions were maintained. The fermentation was allowed to proceed for 17.75 and 42 Hr before sampling. The headspace contained 10.2% O₂ and 7.8% CO₂ (PBI Dansensor CheckMate 9900). The cells were removed via centrifugation (Sorvall RC-5B+, G3 rotor) at 13,689 g for 20 minutes. The supernatant was sampled for analysis via HPLC-RID/HPAEC-PAD and the remainder frozen at −78° C. pending analysis of bacteriocin content by mass spectrometry (target Pediocin, structural gene pedA, 4.6 kDa; MKKIEKLTEK EMANIIGGKY YGNGVTCGKH SCSVDWGKAT TCIINNGAMA WATGGHQGNH KC, SEQ ID NO:9). A small subsample of the biomass was resuspended to wash and centrifuged again (Eppendorf 5415 C) at 12 kRPM for 15 minutes. The washed (miniscule) biomass pellet was re-suspended and gram stained for confirmation of culture morphology and purity via oil-immersion microscopy.

### Results

The profile for the ISOThrive™ MIMO prebiotic in the presence of Pediococcus acidilactici NRRL B-5727 was unchanged during the fermentation relative to the pre-inoculum media. The Pediococcus acidilactici NRRL B-5727 bacteria apparently consumed all of the residual glucose, fructose and maltose, and then died. The few cells that could be found were, while dead (staining pink), exhibited a morphology conforming to Pediococcus spp. These results indicate that the ISOThrive™ MIMO prebiotic does not stimulate the growth of Pediococcus acidilactici NRRL B-5727.

### Example 6: Pediococcus acidilactici NRRL B-5727 Growth in Media Containing Prebiotin™ FOS Enriched Inulin

The inventors decided to do further BLAST analyses to look for levA and fruA (both transporters), as well as 1-FEH (fructan-β-(2,1)-fructosidase, inulin type), and 6-FEH (fructan-β-(2,6)-fructosidase, kestose or FOS type) proteins. The full list of known genes for fructosidase activities are given in the Kegg database (see website at www.genome.jp/dbget-bin/www_bget?ec: 3.2.1.80). Using these methods, the inventors hypothesized that organisms such as Pediococcus acidilactici should consume fructan, β-(2,6), in particular because this organism has levA and/or fruA type transporters. To test this theory, a fermentation was conducted using a prebiotic FOS composition containing both β-(2,1) (inulin), and β-(2,6) (kestose/levan type) fructans.

### Methods

MRS (Sigma) media was prepared by dissolving 5.575 g MRS in 99.970 g (total) water (18M Ω). The media was autoclaved at 121° C. for 15 min. The media was inoculated with Pediococcus acidilactici NRRL B-5727 (0.5 mL late log-phase culture frozen at −78° C. in 20% glycerol), and incubated at 35° C. for 16 Hr. This culture was the inoculum for the following fermentation.

To a 2 L fermenter (New Brunswick BioFlo/Celligen 115) was added water (18M Ω), 0.960 kg; peptone (meat) 10.006 g; yeast extract, 5.649 g; Prebiotin™ FOS enriched inulin (lot #160120), 25.043 g; MnSO₄—H₂O, 0.01236 g; MgSO₄, 0.12790 g; FeSO₄·7H₂O, 0.01235 g; KH₂PO₄, 2.924 g; NaCl, 0.01207 g, and CaCl₂)-2H₂O, 0.06104 g. The whole was autoclaved at 121° C. for 15 minutes. Once cooled to 35° C., this temperature was maintained using a recirculating water bath. The pH was adjusted to pH 6.6 with NaOH (50% w/w) and this pH was maintained throughout using NaOH (40% w/w). To the fermenter was aseptically added 20 mL late log-phase Pediococcus acidilactici NRRL B-5727 inoculum, and micro-anaerobic (self-blanketing) conditions were maintained. The fermentation was allowed to proceed for 17 and 42 Hr before sampling. The headspace contained 9.89% O₂ and 9.0% CO₂ (PBI Dansensor CheckMate 9900). The cells were removed via centrifugation (Sorvall RC-5B+, G3 rotor) at 13,689 g for 20 minutes. The supernatant was sampled for analysis via HPLC-RID/HPAEC-PAD and the remainder frozen at −78° C. pending analysis of bacteriocin (target pediocin) content by mass spectrometry. A small subsample of the biomass was resuspended to wash and centrifuged again (Eppendorf 5415 C) at 12 kRPM for 15 minutes.

### Results

The washed (miniscule) biomass pellet was re-suspended and gram stained for confirmation of culture morphology and purity via oil-immersion microscopy.

At 17 Hr all of the DP 3 (kestose, a type of fructooligosaccharides (FOS)) had been consumed. Hence, although the Pediococcus acidilactici NRRL B-5727 did not metabolize MIMOs as shown in Example 5, the Pediococcus acidilactici NRRL B-5727 did metabolize the Prebiotin™ FOS enriched inulin. Accordingly, identification of microorganism species via rDNA analysis and evaluation of the types of enzymes available to the identified species of microorganisms can be used to identify which probiotic will support the growth and metabolism of the identified microorganism.

### Example 7: Lactobacillus plantarum NRRL-B-4496Growth in Media Containing ISOThrive™ IMO

This Example illustrates that Lactobacillus plantarum NRRL-B4496 grows in media fortified with ISOThrive™ MIMO. Analysis of the genomic data and subsequent reference for the DexA gene indicated that the organism had a PrbI of 30 for oligo-alpha-1,6-glucosidase, alone indicating that the organism was likely to be able to utilize MIMO as a sole carbon source.

### Methods

MRS (Sigma) media was prepared by dissolving 5.540 g MRS broth in 99.791 g (total) water (18M Ω). The media was autoclaved at 121° C. for 15 min. After cooling, the media was inoculated with Lactobacillus plantarum NRRL-B-4496 (0.5 mL late log-phase culture frozen at −78° C. in 20% glycerol), and incubated at 35° C. for 16 Hr. This culture was the inoculum for the following fermentation.

To a 2 L fermenter (New Brunswick BioFlo/Celligen 115) was added water (18M Ω), 0.960 kg; peptone (meat) 10.027 g; yeast extract, 5.541 g; ISOThrive™ IMO (lot #160120), 25.116 g; MnSO₄—H₂O, 0.01297 g; MgSO₄, 0.12796 g; FeSO₄·7H₂O, 0.01366 g; KH₂PO₄, 2.93590 g; NaCl, 0.01355 g, and CaCl₂)-2H₂O, 0.06377 g. The media was autoclaved at 121° C. for 15 minutes. Once cooled to 35° C., this temperature was maintained using a recirculating water bath. The pH was adjusted to pH 6.6 with NaOH (50% w/w) and this pH was maintained throughout using NaOH (40% w/w).

To the fermenter was aseptically added 20 mL late log-phase Lactobacillus plantarum NRRL-B-4496 inoculum, and micro-anaerobic (self-blanketing) conditions were maintained. The fermentation was allowed to proceed for 34 Hr before sampling. The headspace contained 8.4% 02 and 5.7% CO₂ (PBI Dansensor CheckMate 9900). The cells were removed via centrifugation (Sorvall RC-5B+, G3 rotor) at 13,689 g for 20 minutes. The supernatant was sampled for analysis via HPLC-RID/HPAEC-PAD and the remainder frozen at −78° C. pending analysis of bacteriocin content by mass spectrometry. The target was plantaricin M and Z, 6.7956 and 7.1922 kDa (see, Amina et al., Int J Biol Chem 9:46-58 (2015)). A small subsample of the biomass was resuspended to wash and centrifuged again (Eppendorf 5415 C) at 12 kRPM for 15 minutes. The washed biomass pellet was re-suspended and gram stained for confirmation of culture morphology and purity via oil-immersion microscopy.

### Results

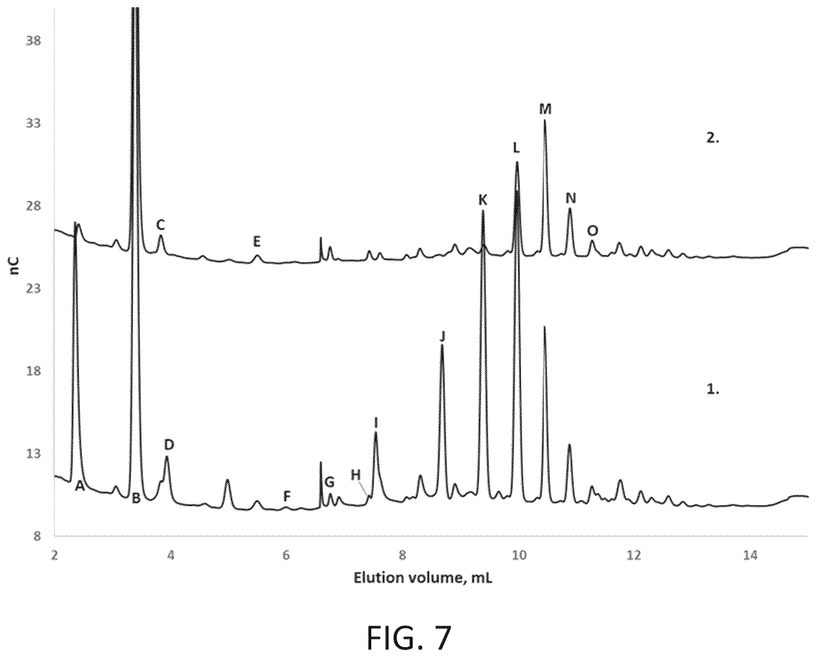

Most of the tested prebiotic ISOThrive™ MIMO composition had been consumed by 34 hours after inoculation with Lactobacillus plantarum NRRL-B-4496:67.2% of the MIMO had been consumed and the remaining material was DP >4 (compare pre-inoculation media Trace 1 with the 34 Hr fermentation media Trace 2 in FIG. 7). In particular, the carbohydrate profiles of media are shown (HPAEC-PAD) in FIG. 7 for the pre-inoculum media and a sample of media taken after 34 Hr fermentation. The components detected in the media were A, mannitol; B, L-arabinose (IS); C, unknown; D, glucose; E, leucrose; F, isomaltose; G, isomaltotriose; H. isomaltotetraose; I, maltose, and J-O, PAN-type IMO (MIMO) DP 3-8. As illustrated, most of the maltose (I), and lower DP MIMOs (e.g., peaks J and K) disappear by 34 Hr. fermentation.

Further analysis showed that the culture was pure and late log phase bacteria had morphology conforming to Lactobacillus spp.

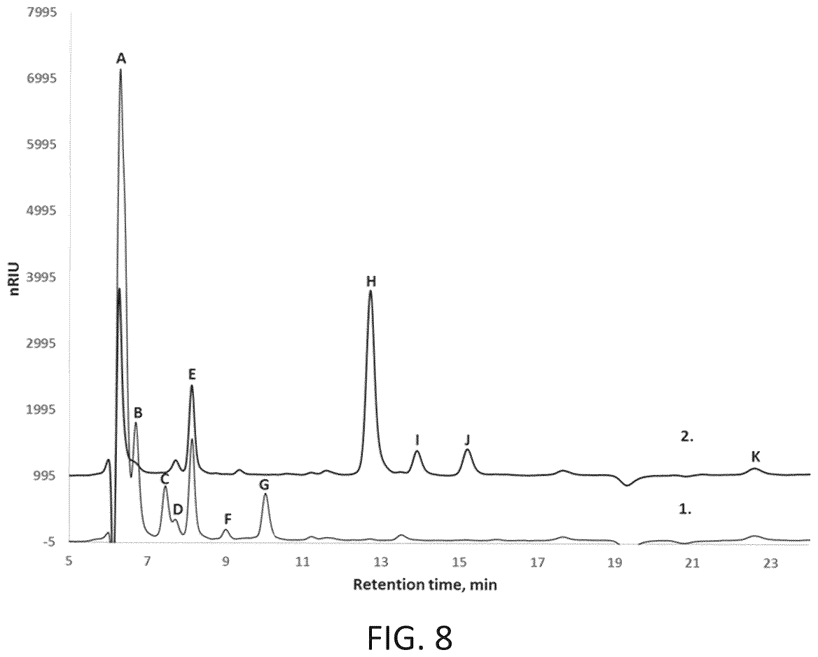

When ISOThrive™ MIMO was the carbon source, pyruvate metabolism was observed, where primarily lactate and traces of formate, acetate, and ethanol were produced (FIG. 8). In particular, FIG. 8 illustrates the metabolic products (as detected by HPLC-RID) of L. plantarum NRRL B-4496 when ISOThrive™ MIMO is a sole carbon source. Trace 1: Pre-inoculation media. Trace 2: media after 34 Hr fermentation. The components detected were: A, MIMO DP >3; B, panose; C, maltose; D, leucrose; E, unknown acid from media; F, glucose; G, mannitol; H, lactate; I, formate; J, acetate, and K, ethanol. As illustrated, the pre-inoculation media (Trace 1) has little or no lactate (peak H), formate (peak I) or acetate (peak J). But significant amounts of lactate (peak H), formate (peak I) or acetate (peak J) are detected after 34 Hr fermentation of ISOThrive™ MIMO by Lactobacillus plantarum NRRL-B-4496.

### Example 8: MIMO Prebiotics Reduce or Eliminate Gastrointestinal Reflux

This Example describes two cases of near complete resolution of gastroesophageal reflux disease (GERD) symptoms after several weeks of daily consumption of a specific maltosylisomaltooligosaccharide (MIMO) fermented prebiotic soluble fiber. A 54-year-old white male and a 69-year-old white female, both with long-standing physician-diagnosed GERD and treated previously with various standard pharmacological therapies, began daily ingestion of a MIMO fermented prebiotic soluble fiber supplement. Over 2-3 months, they both noted nearly complete symptom resolution despite discontinuation of standard therapies.

### Case Descriptions

Spontaneous, unsolicited reports were received from GERD patients who were consuming a new, commercially available fermented MIMO prebiotic (brand name, ISOThrive™). All of the patients reported a history of GERD symptoms of various durations, which had been treated with antacids, H₂ blockers, or proton-pump inhibitors, with variable symptom control.

All reported symptom improvement or resolution after 2-8 weeks of daily ingestion of this MIMO prebiotic supplement. Moreover, all had decreased or discontinued their GERD medications without return of symptoms. Importantly, the product had not previously been marketed as having any effect on GERD, implying that the patients were unlikely to have been influenced by suggestion and accordingly unlikely to be reporting the result of a placebo effect.

Customers in the ISOThrive database were subsequently queried about their experience of GERD symptoms before and after ingesting MIMO prebiotic supplementation for several weeks. The results are tabulated in Table 6. Of 24 GERD patients queried, 21 (88%) experienced symptom improvement after MIMO supplementation, and 17% experienced complete symptom resolution.

TABLE 6

| Reported Effect on GERD Symptoms | Subjects | % |
|---|---|---|
| No Improvement | 3 | 13 |
| Small improvement | 6 | 25 |
| Moderate improvement | 3 | 13 |
| Much Improved | 8 | 33 |
| Resolved | 4 | 17 |
| Subjects, Total | 24 | |

Two representative cases from the queried patients are presented below. Histories reflect the patients' accounts rather than chart reviews.

### Subject A:

Subject A is a 54-year-old white male, with a history of hypertension and hyperlipidemia, on lisinopril (40 mg/day) and simvastatin (40 mg/day). He first developed recurrent heartburn symptoms in 2011, described as “sour stomach” accompanied by burning substernal chest pain. His symptoms were reliably precipitated by red wine, coffee, and peanut butter, and occurred at least 2 days per week.

Shortly after symptom onset he began taking antacids, and experienced symptom improvement but not resolution. In 2012, his primary care physician diagnosed GERD and prescribed omeprazole (20 mg/day), and the patient subsequently experienced improved symptom control. In 2013, he underwent an upper gastrointestinal series and an esophagogastroduodenoscopy that revealed a hiatal hernia and esophageal scarring consistent with chronic GERD. In 2014, he began experiencing intermittent nausea. His omeprazole dose was doubled to 20 mg b.i.d., followed by nearly complete resolution of GERD symptoms but no alleviation of nausea. Further evaluation by his primary care physician revealed slightly elevated liver function tests. He was subsequently referred to a gastroenterologist and, after evaluation and testing, was told he had a “fatty liver” and a “sluggish gallbladder,” the latter diagnosed by HIDA scan. Gastroenterological consultation also confirmed the diagnosis of GERD, and omeprazole (20 mg b.i.d.) and intermittent antacids were continued, with excellent symptom control.

In early 2016, he became concerned about the long-term use of proton pump inhibitors, and discontinued omeprazole. Within days, his prior heartburn symptoms returned at their previous severity, but with an increased frequency of several times per week. After several weeks of poor symptom control with intermittent antacids, he added intermittent omeprazole (20 mg several times per week), and experienced improved symptom control.

About 2 months after symptom recurrence, after hearing about the purported general health benefits of prebiotic soluble fiber, he began taking a daily MIMO dietary supplement. Over 3 months, he noted a gradual decrease in symptom frequency and severity, and again discontinued his omeprazole. Since that time, while continuing the MIMO supplement, symptoms occur less often than once per week on average. Symptoms arising are treated successfully with one or two antacid tablets, which is a significant improvement over his pre-MIMO symptom baseline. He has returned to consuming dietary items that previously reliably led to reflux symptoms (e.g., red wine, coffee, and peanut butter) without experiencing symptom exacerbation.

### Subject B:

Subject B is a 69-year-old white female with a remote history of alcoholism, bulimia, and surgically treated endometrial cancer. Past medical history also included shingles, sleep apnea treated with continuous positive airway pressure, fibromyalgia, and depression treated successfully with venlafaxine. Her regimen included a variety of nutritional supplements including a probiotic, primrose oil, magnesium, lysine, turmeric, red yeast rice, and glucosamine.

In 2006, she first experienced the onset of an intermittent burning sensation in the throat accompanied by belching, burning substernal chest pain, nausea, and intermittent constipation. She saw her personal physician and was started on omeprazole (20 mg/day) and intermittent antacids, with improvement in the frequency and severity of symptoms. After about a month, fearing the long-term side effects of omeprazole, she discontinued the drug, with a subsequent increase in symptom frequency, which were partially controlled by intermittent antacids. This continued into 2015, when she experienced an increase in the frequency of symptoms (daily), reliably precipitated by certain foods including bananas and berries. She gradually eliminated the offending foods, with some symptom improvement.

In mid-2016, after hearing about the purported general health benefits of prebiotic soluble fiber, she began taking a daily MIMO dietary supplement. Over 2 months she noted gradual, complete symptom resolution. At that time, while travelling, she ran out of the MIMO supplement, and shortly thereafter noted the return of daily symptoms. After 2 weeks without the supplement, she began taking it again, and after 7-10 days again noted complete symptom resolution. She remains symptom-free while continuing the daily MIMO supplement. She has normalized her diet, including reintroducing previously offending foods, without symptom exacerbation.

These cases represent the first report of improvement or elimination of GERD symptoms in patients ingesting any type of prebiotic soluble fiber.

### REFERENCES

1. Papagianni, M. and Anastasiadou, S. (2009). “Pediocins: The Bacteriocins of Pediococci. Sources, Production, Properties, and Applications. Microb. Cell Fact. 8 (3), doi: 10.1186/1475-2859-8-3.
2. McAuliffe, O., Ryan, M. P., Ross, R. P., Hill, C., Breeuwer, P. and Abee, T. (1998). “Lacticin 3147, a Broad-Spectrum Bacteriocin Which Selectively Dissapates the Membrane Potential.” Appl. Environ. Microbiol. 64 (2), pp. 439-445.
3. Maldonado, A., Jimenez-Diaz, R. and Ruiz-Barba, J-L. (2004). “Induction of Plantaricin Production in Lactobacillus plantarum NC8 after Coculture with Specific Gram-Positive Bacteria is Mediated by an Autoinduction Mechanism. J. Bacteriol. 186 (5), pp. 1556-1564.
4. Holo, H., Jeknic, Z., Daeschel, M., Stevanovic, S. and Nes, I. F. (2001). “Plantaricin W from Lactobacillus plantarum Belongs to a New Family of Two-peptide Lantibiotics.” Microbiol. 147 (3), pp. 643-651.
5. Herranz, C., and Driessen, A. J. M. (2005). “Sec-Mediated Secretion of Bacteriocin Enterocin by Lactococcus lactis.” Appl. Environ. Microbiol. 71 (4), pp. 1959-1963.
6. Gratia, A. (1925). “Sur un remarquable example d′antagonisme entre deux souches de colibacille”. Compt. Rend. Soc. Biol. 93, pp. 1040-1042.
7. Jones, E., Salin, V. and Williams, G. W. (2005). “Nisin and the Market for Commericial (sic) Bacteriocins.” TAMRC Consumer and Product Research Report No. CP-01-05 (see webpage at ageconsearch.umn.edu/bitstream/90779/2/CP % 2001%2005%20Nisin %20Report.pdf).
8. Zorn, H. and Czermak, P. (2014). “Biotechnology of Food and Feed Additives.” Advances in Biochemical Engineering/Biotechnology 143, Scheper, T. ED. Springer, Heidelberg-New York-Dordrecht-London. ISBN #978-3-662-43760-5, DOI 10.1007/978-3-662-43761-2. pp. 32-34.
9. Reeves, P. (1979). “The Concept of Bacteriocins.” Zentralbl. Bakteriol. Orig. A. 244 (1), pp. 78-89.
5. Fox, P. F., Guinee, T. P., Cogan, T. M. and McSweeney, P. L. H. (2000). “Fundamentals of Cheese Science.” Aspen Publishers, Inc. Gaithersberg, MD. ISBN 0-8342-1260-9, pp. 92-95.
10. Stevens, K. A., Sheldon, B. W., Klapes, N. A. and Klaenhammer, T. R. (1991). “Nisin Treatment for Deactivation of Salmonella Species and Other Gram Negative Bacteria.” Appl. Environ. Microbiol. 57 (12), pp. 3613-3615.
11. Kamarajan, P., Hayami, T., Matte, B., Liu, Y., Danclu, T., Ramamoorthy, A., Worden, F., Kapila, S. and Kapila, Y. (2015). “Nisin ZP, a Bacteriocin and Food Preservative, Inhibits Head and Neck Cancer Tumorigenesis and Prolongs Survival.” PLOS ONE, DOI: 10.1371/journal.pone.0131008.
12. Shin, J. M., Gwak, J. W., Kamarajan, P., Fenno, J. C., Rickard, A. H. and Kapila, Y. L. (2015). “Biomedical Applications of Nisin.” J. Appl. Microbiol. 120, pp. 1449-1465.
13. Kamarajan, P., Hayami, T., Matte, B., Liu, Y., Danciu, T., Ramamoorthy, A., Worden, F., Kapila, S. and Kapila, Y. (2015). “Nisin ZP, a bacteriocin and Food Preservative Inhibits Head and Neck Cancer Tumorigenesis and Prolongs Survival.” PLOS ONE DOI: 10.1371/journal.pone.0131008, pp. 1-20.
14. Joo, N. E., Ritchie, K., Kamarajan, P., Miao, D. and Kapila, Y. (2012). “Nisin, an Apoptotic Bacteriocin and Food Preservative, Attenuates HNSCC Tumorigenesis via CHAC1. Cancer Medicine 1 (3), pp. 295-305.
15. Kaur, S. and Kaur, S. (2015). “Bacteriocins as Potential Anticancer Agents.” Front. Pharmacol. 6 (272), pp. 1-11.
16. Zschuttig, A., Zimmerman, K., Blom, J., Goesmann, A., Pohlmann, C. and Gunzer, F. (2012). “Identification and Characterization of Microcin S, a New Antibacterial Peptide Produced by Probiotic Escherichia coli G3/10.” PLOS ONE 7 (3):e33351; doi: 10.1371/journal.pone.0033351.
17. Hetz, C., Bono, M. R., Barros, L. F. and Lagos, R. (2002). “Microcin E492, a Channel-Forming Bacteriocin from Klebsiella pneumoniae, Induced Apoptosis in some Human Cell Lines.” Proc. Natl. Acad. Sci. U.S.A. 99 (5), pp. 2696-2701.
18. Piva, A. and Headon, D. R. (1994). “Pediocin A, a Bacteriocin Produced by Pediococcus pentosaceus FBB61.” Microbiology 140 (4), pp. 697-702.
19. Simha, B. V., Sood, S. K., Kumariya, R. and Garsa, A. K. (2012). “Simple and Rapid Purification of Pediocin PA-1 from Pediococcus pentosaceus NCDC 273 Suitable for Industrial Application. Microbiol. Res. 167 (9), pp. 544-549.
20. Chikindas, M. L., Garcia-Garcera, M. J., Driessen, A. J. M., Ledeboer, A. M., Nissen-Meyer, J., Nes, I. F., Abee, T., Kinings, W. N. and Venema, G. (1993). “Pediocin PA-1, a Bacteriocin from Pediococcus acidilactici PAC1.0, Forms Hydrophilic Pores in the Cytoplasmic Membrane of Target Cells.” Appl. Env. Microbiol. 59 (11), pp. 3577-3584.
21 Rodriguez, J. M., Matrinez, M. I. and Kok, J. (2002). “Pediocin PA-1, a Wide-Spectrum Bacteriocin from Lactic Acid Bacteria.” Crit. Rev. Food Sci. 42 (2), pp. 91-121.
22. Lianou, A. and Sameli, J. (2014). “Addition to Termalized Milk of Lactococcus lactis subsp. cremoris M104, a Wild Nisin-A Producing Strain, Replaces the Natural Antilisterial Activity on the Autochthonous Raw Milk Microbiota Reduced by Thermalization.” J. Food. Prot. 77 (8), pp. 1289-1297.
23. Delves-Broughton, J. (1990). “Nisin and its Application as a Food Preservative.” Int. J. Dairy Technol. 43 (3), pp. 73-76.
24. De Vos, W. M., Mulders, J. W., Siezen, R. J., Hugenholtz, J. and Kuipers, O. P. (1993). “Properties of Nisin Z and Distribution of its Gene, nisZ, in Lactococcus lactis.” Appl. Env. Microbiol. 59 (1), pp. 213-218.
25. Shin, J. M., Gwak, J. W., Kamarajan, P., Fenno, J. C., Rickard, A. H. and Kapila, Y. L. (2016). “Biomedical Applications of Nisin.” J. Appl. Microbiol. 120 (6), pp. 1449-1465.
26 O'Connor, P. M., O'Shea, E. F. and Guinane, C. M. (2015). “Nisin His a New Nisin Variant Produced by the Gut-Derived Strain Streptococcus hyointestinalis DPC6484.” Appl. Env. Microbiol. 81 (12), pp. 3953-3960.
27. Wirawan, R. E., Klesse, N. A., Jack, R. W. and Tagg, J. R. (2006). “Molecular and Genetic Characterization of a Novel Nisin Variant Produced by Streptococcus uberis.” Appl. Env. Microbiol. 72 (2), pp. 1148-1156.
28. Entian, K-D. and de Vos, W. M. (1996). “Genetics of Subtilin and Nisin Biosynthesis.” Antonie van Leeuwenhoek 69 (2), pp. 109-117.
29. Diep, D. B., Havarstein, L. S., Nissen-Meyer, J. and Nes, I. F. (1994). “The Gene Encoding Plantaricin A, a Bacteriocin from Lactobacillus plantarum C11, is Located on the Same Transcription Unit as an AGR-like Regulatory System.” Appl. Env. Microbiol. 60 (1), pp. 160-166.
30. Maldonado, A., Jumenez-Diaz, R. and Ruiz-Barba, J. L. (2004). “Induction of Plantaricin Production in Lactobacillus plantarum NC8 after Coculture with Specific Gram-Positive Bacteria is Mediated by an Autoinduction Mechanism.” J. Bacteriol. 186 (5), pp. 1556-1564.
31. Holo, H., Jeknic, Z., Daeschel, M., Stevanovic, S. and Nef, I. F. (2001). “Plantaricin W from Lactobacillus plantarum Belongs to a New Family of Two-peptide Lantibiotics.” Microbiology 147 (3), pp. 643-651.
32. Song, D-F., Zhu, M-Y. and Gu, Q. (2014). “Purification and Characterization of Plantaricin ZJ5, a New Bacteriocin Produced by Lactobacillus plantarum ZJ5.” PLOS ONE (see webpage at dx.doi.org/10.1371/journal.pone.0105549)
33 Todorev, S. D., Vaz-Velho, M. and Gibbs, P. (2004). “Comparison of Two Methods for Purification of Plantaricin ST31, a Bacteriocin Produced by Lactobacillus plantarum ST31.” Braz. J. Microbiol. 35 (1), see webpage/dx.doi.org/10.1590/S1517-83822004000100026).
34. Lim, S. W., Koshy, P. and Noni, A. (2016). “Purification, Characterization and Mode of Action of Plantaricin K25 produced by Lactobacillus plantarum.” Food Control 60, pp. 430-139.
35. Holck, A., Axelsson, L., Birkland, S-E., Aukrust, T. and Blom, H. (1992). “Purification and Amino Acid Sequence of Sakacin A, a Bacteriocin from Lactobacillus sake Lb706.” J. Gen. Microbiol. 138, pp. 2715-2720.
36. Simon, L., Fremaux, C., Cenatiempo, Y. and Berjeaud, J. M. (2002). “Sakacin G, a New Type of Antilisterial Bacteriocin.” Appl. Env. Microbiol. 68 (12), pp. 6416-6420.
37. Mathiesen, G., Huehne, K., Kroeckel, L., Axelsson, L. and Eijsink, V. G. (2005). “Characterization of a New Bacteriocin Operon in Sakacin P-producing Lactobacillus sakei, Showing Strong Translational Coupling Between the Bacteriocin and Immunity Genes.” Appl. Env. Microbiol. 71 (7), pp. 3565-3574.
38. Gao, Y., Li, D., Sheng, Y. and Liu, X. (2011). “Mode of Action of Sakacin C2 against Escherichia coli.” Food Control 22 (5), pp. 657-661.
39. Barefoot, S. F. and Klaenhammer, T. R. (1983). “Detection and Acitivity of Lactacin B, a Bacteriocin Produced by Lactobacillus acidophilus.” Appl. Env. Microbiol. 45 (6), pp. 1808-1815.
40. Muriana, P. M. and Klaenhammer, T. R. (1991). “Purification and Partial Characterization of Lactacin F, a Bacteriocin Produced by Lactobacillus acidophilus 11088.” Appl. Env. Microbiol. 57 (1), pp. 114-121.
41. Tabasco, R., Garcia-Cayuela, T., Pelaez, C. and Requena, T. (2009). “Lactobacillus acidophilus La-5 Increases Lactacin B Production When it Senses Live Target Bacteria.” Int. J. Food Microbiol. 132, pp. 109-116.
42. Upreti, G. C. and Hinsdill, R. D. (1975). “Production and Mode of Action of Lactocin 27: Bacteriocin from a Homofermentative Lactobacillus.” Antimicrob. Agents Chemother. 7 (2), pp. 139-145.
43. Turovskiy, Y., Ludenscher, R. D., Aroutcheva, A. A., Faro, S. and Chikindas, M. L. (2009). “Lactocin 160, A Bacteriocin Produced by Vaginal Lactobacillus rhamnosus Targets Cytoplasmic Membranes of the Vaginal Pathogen, Gardnerella vaginalis.” Probiotics Antimicrob. Proteins 1 (1), pp. 67-74.
44. Hastings, J. W., Sailer, M., Johnson, K., Roy, K. L., Vederas, J. C. and Stiles, M. E. (1991). “Characterization of Leucocin A-UAL 187 and Cloning of the Bacteriocin Gene from Leuconostoc gelidum.” J. Bacteriol. 173 (23), pp. 7491-7500.
45. Zacharof, M. P. and Lovitt, R. W. (2012). “Bacteriocins Produced by Lactic Acid Bacteria A Review Article.” APCBEE Procedia 2, pp. 50-56.
46. Makhloufi, K. M., Carre-Mlouka, A., Peduzzi, J., Lombard, C., van Reenen, C. A., Theodore Dicks, L. M. and Rebuffat, S. (2013). “Characterization of Leucocin B-KM432Bz from Leuconostoc pseudomesenteroides Isolated from Boza, and Comparison of its Efficiency to Pediocin PA-1.” PLOS ONE (see webpage at dx.doi.org/10.1371/journal.pone.0070484).
47. Felix, J. A., Papathanasopoulos, M. A., Smith, A. A., von Holy, A. and Hastings, J. W. (1994). “Characterization of Leucocin B-Talla: A Bacteriocin from Leuconostoc carnosum Talla Isolated from Meat.” Current Microbiol. 29, pp. 207-212.
48. Castellano, P., Raya, R. and Vignolo, G. (2003). “Mode of Action of Lactocin 705, a Two-Component Bacteriocin from Lactobacillus casei CRL705.” Int. J. Food. Microbiol. 85 (1-2), pp. 35-43.
49. Mortvedt-Abildgaard, C. I., Nissen-Meyer, J., Jelle, B., Grenov, B., Skaugen, M. and Nes, I. F. (1995). “Production and pH-Dependent Bactericidal Activity of Lactocin S, a Lantibiotic from Lactobacillus sake L45.” Appl. Env. Microbiol. 61 (1), pp. 175-179.
50. Fuchs, S. W., Jaskolla, T. W., Bochmann, S., Kotter, P., Wichelhaus, T., Karas, M., Stein, T. and Entian, K-D. (2011). “Entianin, a Novel Subtilin-like Lantibiotic from Bacillus subtilis subsp. spizizenii DSM 15029 with High Antimicrobial Activity.” Appl. Env. Microbiol. 77 (5), pp. 1698-1707.
51. Jena, P. K., Trivendi, D., Chaudhary, H. and Seshadri, S. (2013). “Bacteriocin PJ4 Active against Enteric Pathogen Produced by Lactobacillus helveticus PJ4 Isolated from Gut Microflora of Wistar Rat (Rattus norvegicus): Partial Purification and Characterization of Bacteriocin.” Appl. Biochem. Biotechnol. 169 (7), DOI: 10.1007/s12010-012-0044-7.
52. Tufail, M., Hussain, S., Malik, F., Mirza, T., Parveen, G., Shafaat, S., Wajid, A., Mahmood, R., Channa, R. A. and Sadiq, A. (2011). “Isolation and Evaluation of Antimicrobial Activity of Bacteriocin Produced by Lactobacillus bulgaricus from Yogurt.” African J. Microbiol. Res. 5 (22), pp. 3842-3847.
53. Michaylova, M., Minkova, S., Kimura, K., Sasaki, T. and Isawa, K. (2007). “Isolation and Characterization of Lactobacillus delbrueckii ssp. bulgaricus and Streptococcus thermophilus from Plants in Bulgaria.” FEMS Microbiol. Lett. 269 (1), pp. 160-169.
54. Bonelli, R. R., Schneider, T., Sahl, H. G. and Wiedemann, I. (2006). “Insights into In Vivo Activities of Lantibiotics from Gallidermin and Epidermin Mode-of-Action Studies.” Antimicrob. Agents Chemother. 50 (4), pp. 1449-1457.
55. Kellner, R., Jung, G., Horner, T., Zahner, H., Schnell, N., Entian, K. D. and Gotz, F. (1988). “Gallidermin: A New Lanthionine-Containing Polypeptide Antibiotic.” Eur. J. Biochem. 177 (1), pp. 53-59.
56. Bierbaum, G., Gotz, F., Peschel, A., Kupke, T., van de Kamp, M. and Sahl, H. G. (1996). “The Biosynthesis of Lantibiotics Epidermin, Gallidermin, Pep5 and Epilancin K7.” Antonie Van Leeuwenhoek 69 (2), pp. 119-127.
57. Gotz, F., Perconti, S., Popella, P., Werner, R. and Schlag, M. (2014). “Epidermin and Gallidermin: Staphylococcal Lantibiotics.” Int. J. Med. Microbiol. 304 (1), pp. 63-71.
58. Velasquez, J. E., Zhang, X. and van der Donk, W. A. (2011). “Biosynthesis of the Antimicrobial Peptide Epilancin 15X and its N-terminal Lactate.” Chem. Biol. 18 (7), pp. 857-867.
59. Brotz, H., Bierbaum, G., Reynolds, P. E. and Sahl, H-G. (1997). “The Lantibiotic Mersacidin Inhibits Peptidoglycan Biosynthesis at the Level of Transglycosylation.” Eur. J. Biochem. 246, pp. 193-199.
60. Altena, K., Guder, A., Cramer, C., Bierbaum, G. (2000). “Biosynthesis of the Lantibiotic Mersacidin: Organization of a Type B Lantibiotic Gene Cluster.” Appl. Env. Microbiol. 66 (6), pp. 2565-2571.
61. McBride, J. (2000). “B12 Deficiency May Be More Widespread Than Thought.” USDA ARS, ars.usda.gov/is/pr/2000/000802.htm
62. Pawlak, R., Parrott, S. J., Raj, S., Cullum-Dugan, D. and Lucus, D. (2013). “How Prevalent is Vitamin B (12) Deficiency Among Vegetarians?” Nutr. Rev. 71 (2), pp. 110-7.

63 Lim, S-H., Choi, J. S. and Park, E. Y. (2001). “Microbial Production of Riboflavin Using Riboflavin Overproducers Ashbya gossypii, Bacillus subtilis, and Candida famate: An Overview.” Biotechnol. Bioprocess Eng. 6, pp. 75-88.
64 LeBlanc, J. G., Milani, C., de Giori, G. S., Sesma, F., van Sinderen, D. and Ventura, M. (2013). “Bacteria as Vitamin Suppliers to their Host: a Gut Microbiota Perspective.” Curr. Opin. Biotechnol. 24(2), pp. 160-8.
65. Perkins, J. B. and Pero, J. (2002). “Vitamin Biosynthesis. In Bacillus subtilis and its closest relatives from genes to cells.” Sonenshein, A., Hoch, J. and Losick, R. EDs. ASM Press, pp. 271-286.
66. Martens, J. H., Barg, H., Warren, M. J. and Jahn, D. (2002). “Microbial Production of Vitamin B12.” Appl. Microbiol. Biotechnol. 58, pp. 275-285.
67 Taranto, M. P., Vera, J. L., Hugenholz, J., de Valdez, G S and Sesma, F. (2003). “Lactobacillus reuteri CRL1098 Produces Cyanocobalamin. J. Bacteriol. 185, pp. 5643-5647.
68. Hollriegl, V., Lamm, L., Rowold, J., Horig, J. and Renz, P. (1982). “Biosynthesis of Vitamin B12 Different Pathways in Some Aerobic and Anaerobic Microorganisms.” Arch. Microbiol. 132, pp. 155-158.
69. Alm, L. (1982). “Effect of Fermentation on B-Vitamin Content of Milk in Sweden.” J. Dairy. Sci. 65, pp. 353-359.
70. Shahani, K. M. and Chandan, R. C. (1979). “Nutritional and Healthful Aspects of Cultured and Culture-Containing Dairy Foods.” J. Dairy Sci. 62, pp. 1685-1694.
71. Champagne, C. P., Tompkins, T. A., Buckley, N. D. and Green-Johnson, J. M. (2010). Effect of Fermentation by Pure and Mixed Cultures of Streptococcus thermophilus and Lactobacillus helveticus on Isoflavone and B-Vitamin Content of a Fermented Soy Beverage.” Food Microbiol. 27, pp. 968-972.
72. McCarthy, R. J. (2004). “Amino Acid Requirements of Dairy Propionibacterium Strains.” Ohio State University.
73. Lan, A. (2008). “Increased Induction of Apoptosis by Propionibacterium freudenreichii TL 133 in Colonic Mucosal Crypts of Human Microbiota-Associated Rats treated with 1,2-dimethylhydrazine.” British J. Nutr. 100 (6), pp. 1251-1259.
74. Jan, G., Balzacq, A-S., Haouzi, D., Roualt, A., Metivier, D., Kroemer, G. and Brenner, C. (2002). “Propioniobacteria Induce Apoptosis of Colorectal Carcinoma Cells via Short-Chaim Fatty Acids acting on Mitochondria. Cell Death & Differentiation 9 (2), pp. 179-188.
75. Courtin, R. and Rul, F. O. (2003). “Interaction between Microorganisms in a Simple Ecosystem: Yogurt Bacteria as a Study Model.” Le Lait 84, pp. 125-134.
76. Simova, E. D., Bashkova, D. M., Angelov, M. P. and Dimitrov, Z. P. (2008). “Bacteriocin Production by Strain Lactobacillus delbrueckii ssp. Bulgaricus BB18 during Continuous Prefermentation of Yogurt Starter Culture and Subsequent Batch Coagulation of Milk. J. Ind. Microbiol. Biotechnol. 35 (6), pp. 559-567.
77. Ghouri, Y. A., Richards, D. M., Rahimi, E. F., Krill, J. T., Jelinek, K. A. and DuPont, A. W. (2014). “Systematic Review of Randomized Controlled Trials of Probiotics, Prebiotics, and Symbiotic in Inflammatory Bowel Disease.” Clin. Exp. Gastroenterol. pp. 473-487.
78. Hegazy, S. K. and El-Bedewy, M. M. (2010). “Effect of Probiotics on Pro-Inflammatory Cytokines and NF-kappaB Activation in Ulcerative Colitis.” World J. Gastroenterol. 16(33), pp. 4145-4151.
79. Rossi, M., Amaretti, A. and Raimondi, S. (2011). “Folate Production by Probiotic Bacteria.” Nutrients 3, pp. 118-134.
80. LeBlanc, J. G., de Giori, G. S., Smid, E. J., Hugenholtz, J. and Sesma, F. (2007). “Folate Production by Lactic Acids Bacteria and Other Food-Grade Microorganisms.” Comm. Curr. Res. Educ. Topics and Trends Appl. Microbiol. Formatex pp. 329-339.
81. Walther, B., Karl, J. P., Booth, S. L. and Boyaval, P. (2013). “Menaquinones, Bacteria, and the Food Supply: The Relevance of Dairy and Fermented Food Products to Vitamin K Requirements.” Adv. Nutr. 4, pp. 463-473.
82. Boundless. “Antibiotics from Prokaryotes.” Boundless Microbiology. Boundless, 26 May 2016.
83. Badet C and Thebaud N B. (2008). Ecology of Lactobacilli in the Oral Cavity: A Review of Literature. Open Microbiol J. 2:38-48. Doi: 10.2174/1874285800802010038.
84. Fan Y P, Chakder S, and Rattan S. (2001). Inducible and neuronal nitric acid synthase involvement in lipopolysaccharide-induced sphincteric dysfunction. Am J Physiol Gastrointest Liver Physiol 280(1):G32-42.
85. Blackett K L, Siddhi S S, Cleary S, Steed H, Miller M H, Macfarlane S, Macfarlane G T, and Dillon J F. (2013). Oesophageal Bacterial Biofilm Changes in Gastro-oesophageal Reflux Disease, Barrett's and Oesophageal Carcinoma: Association or Causality? Aliment Pharmacol Ther 37(11):1084-1092.
86. Harris J K, Fang R, Wagner B D, Choe H N, Kelly C J, Schroeder S, Moore W, Stevens M J, Yeckes A, Amsden K, Kagalwalla A F, Zalewski A, Hirano I, Gonsalves N, Henry L N, Masterson J C, Robertson C E, Leung D Y, Pace N R, Ackerman S J, Furuta G T, and Fillin S A. (2015). Esophageal Microbiome in Eosinophilic Esophagitis. PLOS ONE 10(5):e0128346. Doi: 10.1371/journal.pone.0128346.
87. Kamarajan P, Hayami T, Matte B, Liu Y, Danciu T, Ramamoorthy A, Worden F, Kapila S, and Kapila Y. (2015). Nisin ZP, a Bacteriocin and Food Preservative, Inhibits Head and Neck Cancer Tumorigenesis and Prolongs Survival. PLOS One 10(7):e0131008. doi: 10.1371/journal.pone.0131008.
88. Macfarlane S, Furrie E, Macfarlane G T and Dillon J F. (2007). Microbial Colonization of the Upper Gastrointestinal Tract in Patients with Barrett's Esophagus. 45(1):29-38. Doi: 10.1086/518578.
89. Mathias A and Corthesy B. (2011). N-Glycans on secretory component: Mediators of the interaction between secretory IgA and gram-positive commensals sustaining intestinal homeostasis. Gut Microbes 2(5):287-293.
90. Pei Z, Bini E J, Yang L, Zhou M, Francois F, and Blaser M J. (2004) Bacterial biota in the human distal esophagus. PNAS 101(12). Doi. Pnas.org/cgi/doi/pnas.0306398101.
91. Pei Z, Yang L, Peek R M, Levine Jr S M, Pride D T, and Blaser M J. (2005). Bacterial biota in reflux esophagitis and Barrett's esophagus. World J Gastreoenterol 11(46):7277-7283
92. Rieder F, Cheng L, Harnett K M, Chak A, Cooper G S, Isenberg G, Ray M, Katz J A, Catanzaro, O'Shea R, Post A B, Wong R, Sivak M V, McCormick T, Phillips M, West G A, Willis J E, Biancani P, and Fiocchi C. (2007). Gastroesophageal Reflux Disease-Associate Esophagitis Induces Endogenous Cytokine Production Leading to Motor Abnormalities. Gastroenterol 132(1):154-165
93. Slizova M, Nemcova R, Madar M, Hadryova J, Gancarcikova S, Popper M, and Pistl J. (2015). Analysis of biofilm formation by intestinal lactobacilli. Can J Microbiol
94. Stern N J, Svetoch E A, Eruslanov B V, Perelygin V V, Mitsevich E V, Mitsevich I P, Pokhilenko V D, Levchuk V P, Svetoch O E, and Seal B S. (2006). Isolation of a Lactobacillus salivarius Strain and Purification of Its Bacteriocin, Which Is Inhibitory to Campylobacter jejuni in the Chicken Gastrointestinal System. Antimicrob Agents Chemother 50(9):3111-3116. Doi: 10.1128/AAC.00259-06.
95. Yang L, Francois F, and Pei Z. (2012). Molecular Pathways: Pathogenesis and clinical implications of microbiome alteration in esophagitis and Barrett's esophagus. Clin Cancer Res February 16. Doi: 10.1158/1078-0432.CCR-11-0934.
96. Yang L, Chaudhary N, Baghdadi J, and Pei Z. (2014). Microbiome in Reflux Disorders and Esophageal Adenocarcinoma. Cancer J 20(3):207-210. Doi: 10.1097/PPO.0000000000000044.
97. Yen C H, Tseng Y H, Kuo Y W, Lee M C, and Chen H L. (2011). Long-term supplementation of isomalto-oligosaccharides improved colonic microflora profile, bowel function, and blood cholesterol levels in constipated elderly people—a placebo-controlled, diet-controlled trial. Nutrition 27(4):445-450. Doi: 10.1016/j.nut.2010.05.012.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements describe various features of the invention.

### Statements:

1. A method comprising:
   assaying a fecal, stomach, or upper gastrointestinal sample from a subject to identify classes or types of microorganisms in the sample;
   identifying types of carbohydrate-metabolizing enzymes encoded in genomes of one or more of the classes or types of microorganisms in the sample;
   selecting one or more class(es) or type(s) of microorganism(s) to foster or inhibit in the gut of the subject; and
   making a prebiotic composition that fosters or inhibits the one or more class(es) or type(s) of selected microorganism(s).
2. A method comprising:
   administering a prebiotic to a subject, wherein the prebiotic has a composition prepared by:
      assaying a fecal, stomach, or upper gastrointestinal sample from the subject to identify classes or types of microorganisms in the sample;
      identifying types of carbohydrate-metabolizing enzymes encoded in genomes of one or more of the classes or types of microorganisms in the sample;
      selecting one or more class(es) or type(s) of microorganism(s) to foster or inhibit in the gut of the subject; and
      making a prebiotic composition that fosters or inhibits the one or more class(es) or type(s) of selected microorganism(s).
3. The method of statement 1 or 2, wherein assaying a sample comprises isolation of nucleic acids from the sample, sequencing sample nucleic acids, isolation of protein from the sample, incubation of one or more antibody with sample proteins, or a combination thereof.
4. The method of statement 1, 2 or 3, wherein assaying a sample comprises polymerase chain reaction, primer extension, nucleic acid sequencing, or a combination thereof.
5. The method of statement 1-3 or 4, wherein assaying a sample comprises determining ribosomal RNA sequences, determining ribosomal DNA sequences, determining carbohydrate-metabolizing enzyme sequences, or a combination thereof.
6. The method of statement 1-4 or 5, wherein assaying a sample comprises sequencing sample ribosomal RNAs, sequencing carbohydrate-metabolizing enzyme gene sequences, or a combination thereof.
7. The method of statement1-5 or 6 wherein assaying a sample comprises sequencing sample 16S ribosomal RNAs, sequencing 16S ribosomal DNAs, sequencing 23S ribosomal RNAs, sequencing 23S ribosomal DNAs, or a combination thereof.
8. The method of statement 1-6 or 7, wherein identifying types of carbohydrate-metabolizing enzymes comprises identifying types of carbohydrate-metabolizing enzyme sequences in one or more of the classes or types of microorganisms in the sample.
9. The method of statement 1-7 or 8, wherein identifying types of carbohydrate-metabolizing enzymes comprises identifying types of carbohydrate-metabolizing enzyme sequences missing from sample microorganisms that produce useful products.
10. The method of statement 1-8 or 9, wherein identifying types of carbohydrate-metabolizing enzymes comprises identifying types of carbohydrate-metabolizing enzyme sequences in sample microorganisms that may provide products that can stimulate the growth or metabolism of other microorganisms in the sample.
11. The method of statement 1-9 or 10, wherein identifying types of carbohydrate-metabolizing enzymes comprises sequencing one or more genomic carbohydrate-metabolizing enzyme sequence(s) of the one or more of the class(es) or type(s) of microorganisms in the sample.
12. The method of statement 1-10 or 11, further comprising identifying one or more condition(s) or disease(s) in the subject.
13. The method of statement 1-11 or 12, wherein selecting one or more class(es) or type(s) of microorganism(s) to foster or inhibit in the gut of the subject comprises identifying whether any of the microorganism can synthesize one or more bacteriocins, short chain fatty acids (SCFAs), vitamins, anti-cancer agents, antibiotics, neuromodulators, co-factors, or combinations thereof.
14. The method of statement 1-12 or 13, wherein selecting one or more class(es) or type(s) of microorganism(s) to foster or inhibit in the gut of the subject comprises performing an assay or test to determine whether one or more of the microorganism(s) can synthesize one or more bacteriocins, short chain fatty acids (SCFAs), vitamins, anti-cancer agents, antibiotics, neuromodulators, co-factors, or combinations thereof.
15. The method of statement 1-13 or 14, wherein selecting one or more class(es) or type(s) of microorganism(s) to foster or inhibit in the gut of the subject comprises culturing one or more of the microorganism(s) and testing whether the one or more of cultured microorganism(s) can synthesize one or more bacteriocins, short chain fatty acids (SCFAs), vitamins, anti-cancer agents, antibiotics, neuromodulators, co-factors, or a combination thereof in the culture media.
16. The method of statement 1-14 or 15, wherein selecting one or more class(es) or type(s) of microorganism(s) to foster or inhibit in the gut of the subject comprises identifying whether one or more of the microorganism genome(s) encode one or more bacteriocins, anti-cancer agents, antibiotics, neuromodulators, co-factors, enzymes that make short chain fatty acids (SCFAs), enzymes that make one or more vitamins, or combinations thereof.
17. The method of statement 1-15 or 16, wherein selecting one or more class(es) or type(s) of microorganism(s) to foster or inhibit in the gut of the subject comprises identifying which microorganism(s) can improve the subject's disease or condition.
18. The method of statement 1-15 or 16, wherein selecting one or more class(es) or type(s) of microorganism(s) to foster or inhibit in the gut of the subject comprises identifying which microorganism(s) may cause the subject's disease or condition.
19. The method of statement 1-17 or 18, wherein selecting one or more class(es) or type(s) of microorganism(s) to inhibit in the gut of the subject comprises identifying types of carbohydrate-metabolizing enzymes that the one or more class(es) or type(s) of microorganism(s) does not express or synthesize.
20. The method of statement 1-18 or 19, wherein selecting one or more class(es) or type(s) of microorganism(s) to inhibit in the gut of the subject comprises identifying types of carbohydrate-metabolizing enzymes that the one or more class(es) or type(s) of microorganism(s) does not express or synthesize.
21. The method of statements 1-19 or 20, wherein making a prebiotic composition that inhibits the one or more class(es) or type(s) of selected microorganism(s) comprises making a prebiotic composition that is not metabolized by one or more class(es) or type(s) of selected microorganism(s) but is metabolized by another class or type of selected microorganism(s).
22. The method of statement 1-20 or 21, further comprising administering the prebiotic composition to the subject.
23. The method of statement 1-21 or 22, further comprising administering a prebiotic composition comprising one or more compounds of Formula I.

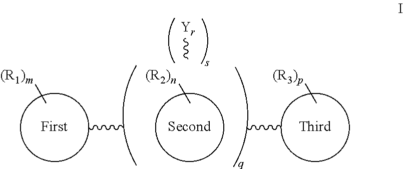

where:
   each First, Second, and Third ring is separately a three-atom, four-atom, five-atom, or six-atom heterocyclic ring with one or two oxygen, sulfur, or nitrogen heteroatoms;
   each Y is an optional monosaccharide or oligosaccharide with r monosaccharides, where each Y has a linkage ( [symbol] ) to a Second ring;
   each [symbol] is separately a linkage between First, Second, and Third ring subunits, as well as linkages between each Y monosaccharide or Y oligosaccharide and a Second ring;
   each m, n, and p is an integer separately selected from any of 2-5;
   q is an integer selected from any of 1-100;
   each r is an integer separately selected from 0-10;
   s is an integer selected from 0-20; and
   each R₁, R₂, and R₃ is separately selected from any of hydrogen, hydroxy, alkoxy, amino, carboxylate, aldehyde (CHO), phosphate or sulfate.
24. The method of statement 23, wherein one or more of the First, Second, or Third rings of Formula I is selected from a five-atom, or six-atom heterocyclic ring.
25. The method of statement 23 or 24, wherein one or more of the First, Second, or Third rings of Formula I has an oxygen or nitrogen heteroatom.
26. The method of statements 23, 24 or 25, wherein the Third ring of Formula I is a monosaccharide.
27. The method of statement 23-25 or 26, wherein the Third ring of Formula I is glucose.
28. The method of statement 23-26 or 27, wherein one or more linkages ( [symbol] ) between the rings or the monosaccharides Formula I is an alpha or beta linkage.
29. The method of statement 23-27 or 28, wherein one or more linkages ( [symbol] ) between the rings or the monosaccharides Formula I is a 1,2-linkage, 1,3-linkage, 1,4-linkage, 1,5-linkage, 1,6-linkage, 2,1-linkage, 2,2-linkage, 2,3-linkage, 2,4-linkage, 2,5-linkage, 2,6-linkage, 3,1-linkage, 3-2, linkage, 3,3-linkage, or a combination thereof.
30. The method of statement 23-28 or 29, wherein less than 20%, or less than 10%, of the total linkages between the First ring, Second rings, Third rings and Y groups can be cleaved by mammalian or avian digestive enzymes in the saliva, stomach and small intestine.
31. The method of statement 23-28 or 29, wherein at least 10%, or at least 30%, or at least 40%, or at least 50%, or at least 60% of the total linkages between the First ring, Second rings, Third rings and Y groups can be cleaved by mammalian or avian digestive enzymes in the saliva, stomach and small intestine.
32. The method of statement 23-29 or 30, wherein less than 20%, or less than 10%, of the total linkages between the First ring, Second rings, Third rings and Y groups are alpha-(1,4) linkages.
33. The method of statement 23-31 or 32, wherein at least 10%, or at least 20%, or at least 30%, or at least 40%, or at least 50%, or at least 55%, or at least 60%, or at least 65%, or at least 70%, or at least 75% of the total linkages between the First ring, Second rings, Third rings and Y groups are alpha(1,2) linkages or alpha-(1,6) linkages.
34. The method of statement 23-31 or 32, wherein at least 10%, or at least 20%, or at least 30%, or at least 40%, or at least 50%, or at least 55%, or at least 60%, or at least 65%, or at least 70%, or at least 75% of the total linkages between the First ring, Second rings, Third rings and Y groups are alpha(1,4) linkages or alpha-(1,6) linkages.
35. The method of statement 23-33 or 34, wherein each m, n, or p is an integer separately selected from any of 3-5.
36. The method of statement 23-34 or 35, wherein each m, n, or p is an integer separately selected from any of 4-5.
37. The method of statement 23-35 or 36, wherein q is an integer is selected from any of 1-20, or an integer is selected from any of 1-15, or an integer is selected from any of 1-10.
38. The method of statement 23-36 or 37, wherein q is larger than s.
39. The method of statement 23-37 or 38, wherein q is an integer of from 2 to 15, or an integer of from 2 to 10, or an integer of from 2 to 7.
40. The method of statement 23-38 or 39, wherein s is an integer of from 1 to 5, or of from 1 to 3, or of from 1 to 2.
41. The method of statement 23-39 or 40, wherein r defines the number of monosaccharides in the optional Y monosaccharide or oligosaccharide.
42. The method of statement 23-40 or 41, wherein r varies from about 0 to 10, or from about 0 to 7, or from about 0 to 5, or from about 0 to 3, or from about 0 to 1.
43. The method of statement 23-41 or 42, comprising administering a composition comprising maltosyl-isomaltooligosaccharides with a mass average molecular weight distribution greater than about 504 or greater than about 640 daltons.
44. The method of statement 43, wherein the composition comprises a mass average molecular weight distribution of about 504 to 20,000 daltons.
45. The method of statement 43 or 44, wherein the maltosyl-isomaltooligosaccharides contain more α-(1-6) glucosyl linkages than α-(1,2), α-(1,3), or α-(1,4) glucosyl linkages.
46. The method of statement 43, 44 or 45, wherein the maltosyl-isomaltooligosaccharides contain at least 10%, or at least 20%, or at least 30%, or at least 40%, or at least 50%, or at least 55%, or at least 60%, or at least 65%, or at least 70%, or at least 75% α-(1,4) glucosyl linkages.
47. The method of statements 43-45 or 46, wherein the composition comprises composition comprising MIMO (DP 3-DP 9); mannitol; fructose; sucrose; maltose; one or more maltodextrins, lactate; glycerol; and acetate.
48. The method of statement 43-46 or 47, wherein the composition comprises composition 3 in the following table, where the values shown are given as %/brix, or % of refractive dry solids.

| Variable | Composition 3 |
|---|---|
| brix: | 18.7 |
| mannitol | 22.49 |
| fructose | 0.02 |
| sucrose | 1.03 |
| maltose | 2.99 |
| MIMO-DP 3 | 6.88 |
| MIMO-DP 4 | 14.07 |
| 1,4-DP3 | 6.29 |
| MIMO-DP 5 | 12.18 |
| MIMO-DP 6 | 5.90 |
| MIMO-DP 7 | 2.33 |
| MIMO-DP 8 | 1.04 |
| 1,4-DP4 | 5.02 |
| MIMO-DP 9 | 0.00 |
| lactate | 16.03 |
| glycerol | 0.35 |
| formate | 0.00 |
| acetate | 4.39 |
| TOTAL: | 101.52 |
| MIMO, %: | 42.40 |
| Purity, %: | 41.77 |
| MWD: | 745.47 |
| Yield %: | 45.76 |

where 1,4-DP3, and 1,4-DP4 are maltodextrins.

49. The method of statement 43-47 or 48, wherein the composition comprises composition comprising MIMO (DP 4-DP 9); mannitol; glucose; sucrose; maltose; panose; 1,4-DP 3 oligosaccharide(s); 1,4-DP 4 oligosaccharide(s); lactate; glycerol; formate; and acetate.
50. The method of statement 43-48 or 49, wherein the composition comprises composition 4 or composition 5 in the following table, where the values shown are given as %/brix, or % of refractive dry solids.

| %/brix | Composition 4 | Composition 5 |
|---|---|---|
| Brix | 25.5 | 26.2 |
| mannitol | 18.28 | 17.35 |
| glucose | 0.44 | 0.93 |
| fructose | 0.00 | 0.00 |
| sucrose | 2.93 | 2.84 |
| maltose | 1.95 | 2.72 |
| panose | 4.65 | 4.43 |
| MIMO-DP4 | 11.33 | 9.96 |
| 1,4-DP3 | 10.66 | 10.23 |
| MIMO-DP5 | 10.29 | 9.05 |
| MIMO-DP6 | 7.08 | 6.82 |
| MIMO-DP7 | 2.94 | 3.12 |
| MIMO-DP8 | 1.85 | 1.69 |
| 1,4-DP4 | 9.93 | 6.33 |
| MIMO-DP9 | 0.00 | 0.00 |
| MIMO-DP10 | 0.00 | 0.00 |
| lactic acid | 8.30 | 8.08 |
| glycerol | 0.24 | 0.23 |
| formic acid | 0.06 | 0.05 |
| acetic acid | 2.92 | 2.86 |
| MIMO | 38.15 | 35.07 |
| MO | 20.59 | 16.56 |
| Total | 93.86 | 86.70 |
| Purity | 40.64 | 40.45 |
| MWD | 795.82 | 790.16 |
| S/M | n/a | n/a |

51. The method of statement 43-49 or 50, wherein the composition comprises the following:
52.
53. The method of statement 1-50 or 51, further comprising treating one or more diseases or conditions in the subject.
54. The method of statements 1-51 or 52, wherein the composition comprises IsoThrive™
55. The method of statement 1-52 or 53, wherein the composition comprises one or more fructo-oligosaccharides; beta-(2,6) oligofructans; inulins; beta-(2,1) oligofructans; beta-1,2 oligosaccharides terminated with glucose; beta-(1,2)-galactooligosaccharides; beta-(1,3)-galactooligosaccharides; beta-(1-4)-galactooligosaccharides; beta-(1,6) galactooligosaccharides; alpha-(1,2)-galactooligosaccharides; alpha-(1,3)-galactooligosaccharides; alpha-(1-4)-galactooligosaccharides; alpha-(1,6) galactooligosaccharides; beta-(1,4) xylooligosaccharides; alpha-(1,4) xylooligosaccharides; hemicelluloses; arabinoxylan; galactomannan; guar gum; acacia gum; arabinogalactan, pectin, amylopectin, or combinations thereof.
56. The method of statement 1-53 or 54, wherein the composition further comprises dietary plant polysaccharides that can be processed by one type of microorganism to foster growth or metabolism of a second type of microorganism.
57. The method of statement 1-54 or 55, wherein the composition further comprises dietary plant polysaccharides that can be processed by B. thetaiotaomicron to foster E. rectale synthesis of butyrate.
58. The method of statement 1-55 or 56, wherein one or more of the selected microorganism(s) synthesizes one or more bacteriocins, short chain fatty acids, vitamins, anti-cancer agents, antibiotics, neuromodulators, co-factors, or combinations thereof.
59. The method of statement 1-56 or 57, further comprising administering the prebiotic composition to the subject.
60. The method of statement 1-57 or 58, further comprising administering the prebiotic composition to the subject from whom the sample was obtained.
61. The method of statement 1-58 or 59, further comprising treating one or more diseases or conditions in the subject selected from a cancer, a pre-cancerous condition, a pre-cancerous propensity, diabetes, type 2 diabetes, an autoimmune disease, a vitamin deficiency, a mood disorder, degraded mucosal lining, ulcerative colitis, digestive irregularity, irritable bowel syndrome, acid reflux, constipation, or a combination thereof.
62. The method of statement 1-59 or 60, further comprising treating a cancer, a pre-cancerous condition, or a pre-cancerous propensity in the subject.
63. The method of statement 1-60 or 61, further comprising treating gastrointestinal reflux in the subject.

The specific compositions and methods described herein are representative, exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a compound” or “a catalyst” or “a ligand” includes a plurality of such compounds, catalysts or ligands, and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72 (b) to allow the reader to quickly ascertain the nature and gist of the technical disclosure. The Abstract is submitted with the understanding that it will not be used to interpret or limit the scope or meaning of the claims.

1.-22. (canceled)

23. A method of treating or ameliorating a condition selected from degraded mucosal lining, intestinal wall inflammation, ulcerative colitis, and a digestive irregularity in a subject, the method comprising:
obtaining a fecal sample from the subject;
sequencing nucleic acids from the fecal sample to identify a plurality of microorganisms present in the fecal sample and to obtain genomic sequences of the plurality of microorganisms;
identifying, from the genomic sequences, for one or more of the plurality of microorganisms, one or more carbohydrate-active enzymes and/or carbohydrate transport proteins encoded by the microorganism;
identifying, from the plurality of microorganisms, one or more target microorganisms that encode one or more enzymes associated with production of a short-chain fatty acid;
determining a hierarchy of carbohydrate preferences for the one or more target microorganisms and for one or more non-target microorganisms in the fecal sample, based at least on the identified carbohydrate-active enzymes or carbohydrate transport proteins;
selecting a carbohydrate formulation comprising at least one carbohydrate substrate that is metabolizable by the one or more target microorganisms, wherein the carbohydrate formulation is selected so as to reduce carbohydrate competition for the one or more target microorganisms by the one or more non-target microorganisms; and
administering the carbohydrate formulation to the subject in an amount effective to increase growth or metabolic activity of the one or more target microorganisms and thereby increase colonic production of the short-chain fatty acid in the subject.

24. The method of claim 23, wherein the short-chain fatty acid comprises butyrate, acetate, propionate, or a combination thereof.

25. The method of claim 23, wherein the short-chain fatty acid comprises butyrate, and wherein the one or more target microorganisms comprise Eubacterium rectale, Roseburia spp., Faecalibacterium prausnitzii, Clostridium butyricum, Clostridium acetobutylicum, Clostridium beijerinkii, or a combination thereof.

26. The method of claim 23, wherein the carbohydrate formulation comprises a plant polysaccharide that is metabolizable by Bacteroides thetaiotaomicron to produce a product that is metabolizable by Eubacterium rectale to produce butyrate.

27. The method of claim 23, wherein the carbohydrate formulation comprises one or more of amylopectin, pectin, arabinoxylan, galactomannan, guar gum, acacia gum, arabinogalactan, xylooligosaccharides, galactooligosaccharides, fructooligosaccharides, and maltosyl-isomaltooligosaccharides.

28. The method of claim 27, wherein the carbohydrate formulation comprises maltosyl-isomaltooligosaccharides having a majority of alpha-(1,6) glucosyl linkages.

29. The method of claim 23, wherein the carbohydrate-active enzymes or carbohydrate transport proteins comprise one or more of glycoside hydrolases, glycosyl transferases, polysaccharide lyases, carbohydrate esterases, dehydrogenases, alpha-glucosidases, oligo-alpha-1,6-glucosidases, alpha-1,6-glucosidases, beta-glucosidases, fructosidases, galactosidases, sucrases, sucrose-isomaltases, invertases, glucuronidases, glucose oxidases, maltases, amylases, isoamylases, beta-phosphoglucomutases, dextranases, pullulanases, mutanases, sialidases, glucosaminases, galactosaminases, xylanases, cellulases, carbohydrate transporters, and a phosphoenolpyruvate: carbohydrate phosphotransferase system.

30. The method of claim 23, wherein determining the hierarchy of carbohydrate preferences comprises weighting matches of sequences from the genomic sequences to reference carbohydrate-active enzyme sequences by match quality.

31. The method of claim 23, wherein selecting the carbohydrate formulation comprises including a carbohydrate substrate that is metabolizable by a microorganism identified in the fecal sample to produce a breakdown product that is metabolizable by the one or more target microorganisms.

32. The method of claim 23, further comprising, after administering the carbohydrate formulation, obtaining a second fecal sample from the subject, sequencing nucleic acids from the second fecal sample, and modifying the carbohydrate formulation based on a change in abundance of the one or more target microorganisms.

33. The method of claim 23, wherein the sequencing comprises shotgun metagenomic sequencing.

34. The method of claim 23, wherein the condition is associated with reduced colonic short-chain fatty acid production in the subject.

35. A method of preparing a carbohydrate composition for a subject, the method comprising:
obtaining a fecal sample from the subject;
sequencing nucleic acids from the fecal sample to identify a plurality of microorganisms present in the fecal sample and to obtain genomic sequences of the plurality of microorganisms;
identifying, from the genomic sequences, carbohydrate-active enzymes and/or carbohydrate transport proteins encoded by one or more of the plurality of microorganisms;
identifying one or more target microorganisms in the fecal sample that encode one or more enzymes associated with production of a short-chain fatty acid;
determining a hierarchy of carbohydrate preferences for the one or more target microorganisms and for one or more non-target microorganisms in the fecal sample, based at least on the identified carbohydrate-active enzymes or carbohydrate transport proteins;
selecting, based on the hierarchy of carbohydrate preferences, one or more carbohydrate substrates that are metabolizable by the one or more target microorganisms and that reduce carbohydrate competition for the one or more target microorganisms by the one or more non-target microorganisms; and
preparing a carbohydrate composition comprising the selected one or more carbohydrate substrates.

36. The method of claim 35, wherein the short-chain fatty acid comprises butyrate.

37. The method of claim 35, wherein determining the hierarchy of carbohydrate preferences further comprises using an abundance measure of one or more of the plurality of microorganisms in the fecal sample.

38. The method of claim 35, wherein identifying the carbohydrate-active enzymes comprises identifying one or more of an alpha-1,6-glucosidase and an oligo-alpha-1,6-glucosidase encoded by the one or more target microorganisms, and wherein the carbohydrate composition comprises maltosyl-isomaltooligosaccharides having a majority of alpha-(1,6) glucosyl linkages.

39. A method of treating or ameliorating a condition associated with reduced colonic butyrate production in a subject, the condition selected from degraded mucosal lining, intestinal wall inflammation, and ulcerative colitis, the method comprising:
obtaining a fecal sample from the subject;
sequencing nucleic acids from the fecal sample to identify a plurality of microorganisms present in the fecal sample and to obtain genomic sequences of the plurality of microorganisms;
identifying, from the genomic sequences, one or more target microorganisms that encode one or more enzymes associated with butyrate production;
identifying, from the genomic sequences, carbohydrate-active enzymes and/or carbohydrate transport proteins encoded by the one or more target microorganisms and by one or more non-target microorganisms in the fecal sample;
selecting a carbohydrate formulation comprising at least one carbohydrate substrate that is metabolizable by the one or more target microorganisms based on the identified carbohydrate-active enzymes or carbohydrate transport proteins, wherein the carbohydrate formulation is selected to reduce carbohydrate competition for the one or more target microorganisms by the one or more non-target microorganisms; and
administering the carbohydrate formulation to the subject in an amount effective to increase colonic butyrate production in the subject.

40. The method of claim 39, wherein the carbohydrate formulation comprises a plant polysaccharide that is metabolizable by Bacteroides thetaiotaomicron to produce a product that is metabolizable by Eubacterium rectale to produce butyrate.

41. The method of claim 39, wherein the carbohydrate formulation comprises maltosyl-isomaltooligosaccharides having a majority of alpha-(1,6) glucosyl linkages, and wherein the one or more target microorganisms encode one or more of an alpha-1,6-glucosidase and an oligo-alpha-1,6-glucosidase.

42. The method of claim 39, wherein the one or more target microorganisms comprise Eubacterium rectale, Roseburia spp., Faecalibacterium prausnitzii, Clostridium butyricum, Clostridium acetobutylicum, Clostridium beijerinkii, or a combination thereof.
